(19) United States
(12) Patent Application Publication
(10) Pub. No.: US 2004/0009537 A1
(43) Pub. Date: Jan. 15, 2004

(54) Methods of modulating and of identifying agents that modulate intracellular calcium

(76) Inventors: Jack Roos, San Diego, CA (US); Kenneth Stauderman, San Diego, CA (US); Gonul Velicelebi, San Diego, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 10/342,844

(22) Filed: Jan. 13, 2003

Related U.S. Application Data
(60) Provisional application No. 60/347,459, filed on Jan. 11, 2002.
(60) Provisional application No. 60/401,171, filed on Aug. 2, 2002.
(60) Provisional application No. 60/405,678, filed on Aug. 20, 2002.

(51) Int. Cl. C12Q 1/68; G01N 33/53; G01N 33/567; C12N 5/06

(52) U.S. Cl. 435/7.2; 435/348; 435/6

(57) ABSTRACT

Methods are provided for identifying agents that modulate intracellular calcium. Also provided are methods of modulating calcium within cells and treating disease by modulating intracellular calcium. Also provided are methods of screening for nucleic acids encoding candidate ion transport proteins and methods of identifying nucleic acids encoding ion transport proteins.

### RELATED APPLICATIONS

Benefit of priority under §119(e) is claimed to U.S. Provisional Application Serial No. 60/347,459, filed Jan. 11, 2002, entitled “METHODS OF MODULATING AND OF IDENTIFYING AGENTS THAT MODULATE INTRACELLULAR CALCIUM, to Jack Roos, Kenneth Stauderman and Gönül Velicelebi, and to U.S. Provisional Application Serial Nos. 60/401,171 and 60/405,678 filed Aug. 2, 2002, and Aug. 20, 2002, respectively, entitled “Methods of Modulating and of Identifying Agents that Modulate Intracellular Calcium”, each to Jack Roos, Kenneth Stauderman and Gönül Velicelebi. The subject matter of each of the provisional applications is incorporated by reference in its entirety.

### FIELD OF THE INVENTION

The present invention relates to methods of identifying agents that modulate intracellular calcium. The invention further relates to methods of modulating calcium within cells and treating disease by modulating intracellular calcium. The invention also relates to methods of screening for nucleic acids encoding candidate ion transport proteins and methods of identifying nucleic acids encoding ion transport proteins.

### BACKGROUND OF THE INVENTION

Calcium plays a vital role in cell function and survival. For example, calcium is a key element in the transduction of signals into and within cells. Cellular responses to growth factors, neurotransmitters, hormones and a variety of other signal molecules are initiated through calcium-dependent processes. Many proteins are activated by binding calcium and in turn affect other proteins in signal cascade mechanisms in cells. The normal basal concentration of free calcium in the cytoplasm of cells is about 50-100 nM whereas the extracellular calcium concentration is typically about 2 mM. Therefore, intracellular calcium levels and fluctuations thereof are tightly regulated by cells.

Calcium regulation by cells is accomplished through a variety of mechanisms, some of which are associated with particular cell types. For example, excitable cells, such as muscle and nerve cells in which calcium signals are essential to functions including contraction and transmission of nerve impulses, contain voltage-gated calcium channels spanning the cell membrane. These channels respond to depolarization of the potential difference across the membrane and can open to permit an influx of calcium from the extracellular medium and a rapid increase in intracellular calcium concentrations.

Nonexcitable cells, e.g., blood cells, fibroblasts and epithelial cells, as well as many excitable cells, contain channels that span intracellular membranes and that can open to permit an influx of calcium into the cytoplasm from calcium-storing organelles, such as the endoplasmic reticulum. One such intracellular ion channel is the inositol 1,4,5-trisphosphate (IP₃) receptor located in the membrane of the endoplasmic reticulum. The IP₃ receptor functions as a ligand-gated ion channel that permits passage of calcium upon binding of IP₃ released through hydolysis of membrane phospholipids by activated phospholipase C (PLC). PLC can be activated through agonist binding to a surface membrane G protein-coupled receptor. Activation of the IP₃ receptor results in the release of calcium stored in the endoplasmic reticulum into the cytoplasm. Reduced endoplasmic reticulum calcium concentration resulting from release of calcium therefrom provides a signal for influx of calcium from the extracellular medium into the cell. It appears that this influx of calcium does not rely on voltage-gated plasma membrane channels and does not involve activation of calcium channels by calcium. This calcium influx mechanism has been referred to as capacitative calcium entry (CCE) or store-operated calcium entry. The actual factor that directly activates influx of calcium across the plasma membrane in CCE is unknown, as is the identity of the molecule or molecules that provide for mobilization of calcium across the plasma membrane and into the cell. Because of the vital role that calcium plays in cell function and survival, dysregulation of calcium in cells can have deleterious effects on cell structure and function. Alterations in intracellular calcium homeostasis have been implicated in a variety of diseases.

There is a need, therefore, to elucidate the factors, structures and mechanisms involved in calcium regulation in cells which may be targets for therapeutic intervention in diseases associated with calcium dysregulation. There is also a need for agents that modulate intracellular calcium and methods of identifying such agents as possible therapeutic compounds for treatment of diseases associated with calcium dysregulation.

### SUMMARY

Provided are methods of identifying an agent or a candidate agent that modulates intracellular calcium. The agent is one that modulate any aspect of intracellular calcium, including, but not limited to store-operated calcium entry, resting cytosolic calcium levels and/or calcium levels in or movements of ions into or out of an intracellular organelle. In particular, methods of identifying agents that modulate intracellular calcium by contacting one or more test cells or a portion of a cell with a test agent; monitoring the effect(s) of a test compound on store-operated calcium entry; and identifying a test agent as an agent or candidate agent if it has an effect on store-operated calcium entry are provided. The portion of the cell can be a cell membrane, an organelle, such as nucleus or mitochondrion, or nuclear envelope.

The test cell contains one or more proteins that is (are) at least about 35% homologous to the protein encoded by Drosophila gene CG4536 or CG5842 over at least about 40% of the encoded protein and that provides for store-operated calcium entry. In other embodiments of all methods provided herein a protein that is homologous to the protein encoded by the Drosophila gene is at least about 35% homologous to the protein encoded by Drosophila gene CG4536 or CG5842 over at least about 50%, 51% or 52% of the encoded protein and that provides for store-operated calcium entry with a test agent. In other embodiments, of all methods provided herein a protein that is homologous to the protein encoded by the Drosophila gene is at least about 41% homologous to the protein encoded by Drosophila gene CG4536 or CG5842 over at least about 50%, 51% or 52% of the encoded protein and that provides for store-operated calcium entry with a test agent. In other embodiments, the protein homologous to the protein encoded by the Drosophila gene is selected to be one that does not contain the contiguous sequence of amino acids EWKFAR (SEQ ID 114) and/or EXD(E)CR(K)GXYXXYE (SEQ ID 115), wherein X is any amino acid and an amino acid residue in parentheses is an alternative to the residue immediately preceding it.

Exemplary proteins in the test cells are a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp12), a transient receptor potential protein V3 (TRPV3), a stretch-inhibitable nonselective channel (SIC) protein. The protein that provide for store-operated calcium entry includes proteins that are components, such as subunits of a store-operated calcium channel, and includes proteins that affect, directly or indirectly, the activity, expression or level of such components. Also, the proteins can be from any source, particularly mammalian sources, and include human proteins.

In certain embodiments of the methods the step of monitoring the effects of a test compound on store-operated calcium entry includes the steps of: comparing store-operated calcium entry into one or more test cell(s) in the presence and absence of the test agent or comparing store-operated calcium entry into a test cell and into a control cell in the presence of the test agent. The step of identifying includes identifying a test agent as an agent or candidate agent that modulates intracellular calcium if store-operated calcium entry into the one or more test cells differs in the presence and absence of the test agent or if store-operated calcium entry into a test cell and the control cell differs. A control cell is generally a substantially similar cell to a test cell, but is a cell that contains a different amount, including none, of the one or more proteins that are homologous to the protein encoded by Drosophila gene CG4536 or CG5842 or the such proteins as described herein. The test cells can be any cell, and are typically eukaryotic cells, particularly mammalian cells, including human cells and cells that serve as human disease models. The cells can be primary cells or a cell line. The test cells and control cells in any experiment are generally from the same source.

In other embodiments, monitoring the effect(s) of the test agent on store-operated calcium entry is effected by reducing the calcium level in an intracellular calcium store of one or more test cells; and monitoring the effect of test agent on intracellular and/or extracellular a) ion movement, b) ion flux or c) ion levels of the one or more test cells; and the step of identifying includes identifying a test agent as an agent or candidate agent if it has an effect on a), b) or c). The step of reducing the calcium levels in an intracellular calcium store is performed before, simultaneously with or after the step of contacting one or more test cells with the test agent.

In these methods, the step of monitoring the effect(s) of test agent on store-operated calcium can further include: comparing a), b) or c) in the presence and absence of the test agent or comparing a), b) or c) of a test cell and a control cell in the presence of the test agent; and identifying further includes: identifying a test agent as an agent or candidate agent that modulates intracellular calcium if a), b) or c) of a test cell differ in the presence and absence of the test agent or if a), b) or c) of a test cell and the control cell differ.

In other embodiments of any of the methods provided herein, the step of monitoring the effects of test agent on store-operated calcium includes: determining intracellular or cytoplasmic calcium levels of one or more test cells; and identifying includes: identifying a test agent as an agent or candidate agent if it has an effect on intracellular or cytoplasmic calcium levels of one or more test cells. In these methods, monitoring the effect(s) of test agent on store-operated calcium can further include: comparing the intracellular calcium levels of one or more test cells in the presence and absence of the test agent or the intracellular calcium levels of a test cell and of a control cell in the presence of the test agent; and identifying is effected by identifying a test agent as an agent or candidate agent that modulates intracellular calcium if the intracellular calcium levels of a test cell differ in the presence and absence of the test agent or if the intracellular calcium levels of a test cell and the control cell differ.

In all of the methods provided herein the step of monitoring the effects of test agent on store-operated calcium can include: monitoring intracellular calcium levels as a function of time in one or more test cells to assess fluctuations in intracellular calcium levels; and identifying includes identifying a test agent as an agent or as a candidate agent if it has an effect on intracellular calcium level as a function of time (its time course). In these methods monitoring the effect(s) of test agent on store-operated calcium can further include: comparing the intracellular calcium level time courses of a test cell in the presence and absence of the test agent or the intracellular calcium level time courses of a test cell and a control cell in the presence of the test agent; and identifying includes: identifying a test agent as an agent that modulates intracellular calcium if the intracellular calcium level time courses of a test cell differ in the presence and absence of the test agent or if the intracellular calcium level time courses of a test cell and the control cell differ.

In all of the methods provided herein the step of monitoring the effects of test agent on store-operated calcium can include monitoring the effects of test agent on store-operated calcium by assessing comprises assessing ion flux across the cell plasma membrane of a test cell; and identifying includes identifying a test agent as an agent or candidate if it has an effect on ion flux across the cell plasma membrane of a test cell. In these methods, monitoring the effect(s) of test agent on store-operated calcium can further include comparing the ion flux across the cell plasma membrane of a test cell in the presence and absence of the test agent or the ion flux across plasma membrane of a test cell and a control cell in the presence of the test agent; and identifying can further include identifying a test agent as an agent or candidate agent that modulates intracellular calcium if the ion flux across the cell plasma membrane of a test cell differs in the presence and absence of the test agent or if the ion flux across the plasma membrane of a test cell and the control cell differs.

Also provided are methods for identifying an agent (or candidate agent) that modulates intracellular calcium by monitoring the effects of an agent on store-operated calcium entry in any test system, including in vivo systems, such as cells and organisms that contain a protein homologous to a Drosophila protein as defined herein, and in vitro systems, such as a protein homologous to a Drosophila protein as defined herein, where the agent modulates the activity of, the interaction of, the level of or binds to a protein that provides for store-operated calcium entry; and identifying a test agent as an agent (candidate agent) that modulates intracellular calcium if it has an effect on store-operated calcium entry in the test system.

The protein in the test system is one that has a requisite homology to a protein encoded by Drosophila gene CG4536 or CG5842 as described above and in detail below, and includes the proteins set forth above and throughout the disclosure. As noted above, in embodiments of all methods provided herein a protein that is homologous to the protein encoded by the Drosophila gene is at least about 35% homologous to the protein encoded by Drosophila gene CG4536 or CG5842 over at least about 50%, 51% or 52% of the encoded protein and that provides for store-operated calcium entry with a test agent. In other embodiments, of all methods provided herein a protein that is homologous to the protein encoded by the Drosophila gene is at least about 41% homologous to the protein encoded by Drosophila gene CG4536 or CG5842 over at least about 50%, 51% or 52% of the encoded protein and that provides for store-operated calcium entry with a test agent. In other embodiments, the protein homologous to the protein encoded by the Drosophila gene is selected to be one that does not contain the contiguous sequence of amino acids EWKFAR (SEQ ID 114) and/or EXD(E)CR(K)GXYXXYE (SEQ ID 115), wherein X is any amino acid and an amino acid residue in parentheses is an alternative to the residue immediately preceding it.

Exemplary of these proteins are a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp12), a transient receptor potential protein V3 (TRPV3), a stretch-inhibitable nonselective channel (SIC) protein.

In the methods for identifying an agent (or candidate agent) that modulates intracellular calcium by monitoring the effects of an agent on store-operated calcium entry in any test system, the step of monitoring the effects, includes: comparing store-operated calcium entry into one or more test cell(s) in the presence and absence of the test agent or comparing store-operated calcium entry into a test cell and into a control cell in the presence of the test agent; and the step of identifying is performed by identifying a test agent as an agent that modulates intracellular calcium if store-operated calcium entry into the one or more test cells differs in the presence and absence of the test agent or if store-operated calcium entry into a test cell and the control cell differs. As described above, a control cell is substantially similar cell to a test cell, but contains a different amount, including none, of the one or more proteins that is (are) at least about homologous to the protein encoded by Drosophila gene CG4536 or CG5842 as described above.

In these methods, the step of monitoring the effect(s) of the test agent on store-operated calcium entry, includes: reducing the calcium level in an intracellular calcium store of one or more test cells; and monitoring the effect of test agent on intracellular and/or extracellular a) ion movement, b) ion flux or c) ion levels of the one or more test cells; and identifying comprises identifying a test agent as an agent if it has an effect on a), b) or c). The step of reducing the calcium levels in an intracellular calcium store is performed before, simultaneously with or after the step of contacting one or more test cells with the test agent.

In these methods, the step of, monitoring the effect(s) of test agent on store-operated calcium can further include: comparing a), b) or c) in the presence and absence of the test agent or comparing a), b) or c) of a test cell and a control cell in the presence of the test agent; and identifying further includes: identifying a test agent as an agent that modulates intracellular calcium if a), b) or c) of a test cell differ in the presence and absence of the test agent or if a), b) or c) of a test cell and the control cell differ.

In the methods for identifying an agent (or candidate agent) that modulates intracellular calcium by monitoring the effects of an agent on store-operated calcium entry in any test system, the step of monitoring the effects of a test agent on store-operated calcium includes: determining intracellular or cytoplasmic calcium levels of one or more test cells; and identifying includes: identifying a test agent as an agent if it has an effect on intracellular or cytoplasmic calcium levels of one or more test cells. In these methods, monitoring the effect(s) of test agent on store-operated calcium can further include: comparing the intracellular calcium levels of one or more test cells in the presence and absence of the test agent or the intracellular calcium levels of a test cell and of a control cell in the presence of the test agent; and identifying can include identifying a test agent as an agent that modulates intracellular calcium if the intracellular calcium levels of a test cell differ in the presence and absence of the test agent or if the intracellular calcium levels of a test cell and the control cell differ.

In these methods, monitoring the effects of test agent on store-operated calcium can include: monitoring intracellular calcium levels as a function of time in one or more test cells for fluctuations in intracellular calcium levels; and

identifying can include: identifying a test agent as an agent (or candidate agent) if it has an effect on intracellular calcium level time courses. The step of monitoring the effect(s) of test agent on store-operated calcium can further include: comparing the intracellular calcium level time courses of a test cell in the presence and absence of the test agent or the intracellular calcium level time courses of a test cell and a control cell in the presence of the test agent; and identifying can include:

identifying a test agent as an agent that modulates intracellular calcium if the intracellular calcium level time courses of a test cell differ in the presence and absence of the test agent or if the intracellular calcium level time courses of a test cell and the control cell differ.

In these methods, monitoring the effects of test agent on store-operated calcium can include assessing comprises assessing ion flux across the cell plasma membrane of a test cell; and identifying can include: identifying a test agent as an agent if it has an effect on ion flux across the cell plasma membrane of a test cell.

Monitoring the effect(s) of test agent on store-operated calcium can further include: comparing the ion flux across the cell plasma membrane of a test cell in the presence and absence of the test agent or the ion flux across plasma membrane of a test cell and a control cell in the presence of the test agent; and

identifying can include identifying a test agent as an agent (or candidate agent) that modulates intracellular calcium if the ion flux across the cell plasma membrane of a test cell differs in the presence and absence of the test agent or if the ion flux across the plasma membrane of a test cell and the control cell differs. 30. The method of claim 1 or claim 18, wherein a protein that provides for store-operated calcium entry is a mammalian protein.

Also provided are methods for modulating store-operated calcium entry. These methods can include first contacting a cell that has altered store-operated calcium entry with an agent that modulates the level of, expression of, activity of or molecular interactions of a protein that is homologous to the protein encoded by Drosophila gene CG4536 or CG5842 as described herein. Such proteins generally also provide for store-operated calcium entry. Such contacting effects modulation of store-operated calcium transport into the cell. These methods for modulating can be performed with using the proteins, test cells, control cells and other components as described above for the methods of identifying agents that modulate intracellular calcium.

In other embodiments, methods for identifying a molecule that provides for store-operated calcium entry are provided. These methods include the step of identifying a molecule that interacts with (binds to or otherwise affects the activity, level of or expression of) a protein as defined above and herein that is homologous to the protein encoded by Drosophila and also that provides for store-operated calcium entry, thereby identifying molecules involved in modulating store-operated calcium entry. This methods can further include eliminating, altering or reducing the expression of a gene encoding the protein in a cell and evaluating intracellular calcium. In these methods the protein includes those defined above. The cells in which intracellular calcium is evaluated is, include any cell as described above and herein, including mammalian cells.

The agents identified by the methods provided herein are agents for or candidate agents for treatment of a variety of disorders or diseases, such as neurodegenerative diseases.

### DETAILED DESCRIPTION OF PREFERRED EMBODIMENTS

Definitions

Unless defined otherwise, all technical and scientific terms used herein have the same meaning as is commonly understood by one of skill in the art to which this invention belongs. All patents, patent applications, publications and published nucleotide and amino acid sequences (e.g., sequences available in GenBank or other databases) referred to herein are incorporated by reference. Where reference is made to a URL or other such identifier or address, it is understood that such identifier can change and particular information on the internet can come and go, but equivalent information can be found by searching the internet. Reference thereto evidences the availability and public dissemination of such information.

As used herein, “intracellular calcium” refers to calcium ions located in a cell without specification of a particular cellular location. In contrast, “cytosolic” or “cytoplasmic” with reference to calcium refers to calcium ions located in the cell cytoplasm.

As used herein, “modulation” with reference to intracellular calcium is any alteration in intracellular calcium, including but not limited to alteration of calcium concentration in the cytoplasm and/or intracellular calcium storage organelles, e.g., endoplasmic reticulum, alteration of the kinetics of calcium fluxes into and within cells. Modulation of the response of a cell to reduction in calcium levels of intracellular calcium stores refers to any alteration in the cell's response, including, but not limited to, alterations in calcium entry into the cell and/or alterations in refilling of intracellular calcium stores.

As used herein, an effect on store-operated calcium entry is any alteration of any aspect of store-operated calcium entry, including but not limited to, an alteration of the operation of a store-operated calcium channel and the properties, such as, for example, the kinetics, sensitivites, rate, amplitude, and electrophysiological characterisitics, of calcium flux or movement that occurs in store-operated calcium entry. These aspects can be assessed in a variety of ways including, but not limited to, evaluation of calcium or other ion (particularly cation) levels, movement of calcium or other ion (particularly cation), fluctuations in calcium or other ion (particularly cation) levels, kinetics of calcium or other ion (particularly cation) fluxes and/or transport of calcium or other ion (particularly cation) through a membrane. An alteration can be any such change that is statistically significant. Thus, for example if store-operated calcium entry into a test cell and a control cell is said to differ, such difference is a statistically significant difference.

As used herein, “provides for store operated calcium entry” with respect to a protein means that when expression or activity of the protein in a cell is reduced, altered or eliminated, there is a concomitant or associated reduction, alteration or elimination of store operated calcium entry. Such reduction in expression or activity can occur by virtue of an alteration of expression of a gene encoding the protein or by altering the levels of the protein. A protein that provides for store-operated calcium entry, thus, can be one that participates in store-operated calcium entry or is part of a store-operated calcium entry channel. A protein that participates in store-operated calcium entry can be a protein that is not necessarily a component of the store-operated calcium entry channel but is directly or indirectly associated with its activity, such as, for example, a non pore-forming subunit or ligand or other modulatory or regulatory protein that modulates its activity.

As used herein, a protein that is a component of a store-operated calcium entry channel is a protein that participates in forming the pore or constitutes the pore of the channel.

As used herein, stating that levels of something fluctuate over time typically refers to time periods on the order of seconds to minutes after an initiating event, such as store depletion, but can refer to longer or shorter time periods. The exact time frame for monitoring an event is a function of the particular assay and event, and one of skill in the art can determine the time period empirically.

As used herein, “heterologous” or “foreign” with reference to nucleic acids, cDNA, DNA and RNA are used interchangeably and refer to nucleic acid, DNA or RNA that does not occur naturally as part of the genome in which it is present or which is found in a location(s) or in an amount in the genome that differs from that in which it occurs in nature. It is nucleic acid that has been exogenously introduced into the cell. Thus, heterologous nucleic acid is nucleic acid not normally found in the host genome in an identical context. Examples of heterologous nucleic acids include, but are not limited to, DNA that encodes a gene product or gene product(s) of interest, introduced, for example, for purposes of gene therapy or for production of an encoded protein. Other examples of heterologous DNA include, but are not limited to, DNA that encodes a selectable marker, DNA that encodes therapeutically effective substances, such as anti-cancer agents, enzymes and hormones, and DNA that encodes other types of proteins, such as antibodies.

As used herein, “expression” refers to the process by which nucleic acid, e.g., DNA, is transcribed into mRNA and translated into peptides, polypeptides, or proteins. If the nucleic acid is derived from genomic DNA, expression may, if an appropriate eukaryotic host cell or organism is selected, include splicing of the mRNA.

As used herein, “vector” or “plasmid” refers to discrete elements that are used to introduce heterologous nucleic acids into cells. Typically, vectors are used to transfer heterologous nucleic acids into cells for either expression of the heterologous nucleic acid or for replication of the heterologous nucleic acid. Selection and use of such vectors and plasmids are well within the level of skill of the art.

As used herein, “transformation” or “transfection” refers to the process by which nucleic acids are introduced into cells. Transfection refers to the taking up of exogenous nucleic acid, e.g., an expression vector, by a host cell whether or not any coding sequences are in fact expressed. Numerous methods of transfection are known to the ordinarily skilled artisan. Successful transfection is generally recognized by detection of the presence of the heterologous nucleic acid within the transfected cell, such as, for example, any visualization of the heterologous nucleic acid or any indication of the operation of a vector within the host cell.

As used herein, “injection” refers to the microinjection (use of a small syringe) of nucleic acid into a cell.

As used herein, the amino acids, which occur in the various amino acid sequences appearing herein, are identified according to their well-known, three-letter or one-letter abbreviations (see Table 1). The nucleotides, which occur in the various DNA fragments, are designated with the standard single-letter designations used routinely in the art.

As used herein, amino acid residue refers to an amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224).

Such substitutions may be made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

As used herein, “similarity” between two proteins or nucleic acids refers to the relatedness between the amino acid sequences of the proteins or the nucleotide sequences of the nucleic acids. Similarity can be based on the degree of identity and/or homology of sequences and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)).

As used herein, “homology” with reference to proteins or nucleic acids refers to shared sequence similiarity that takes into account both identical residues and residues that may substitute for one another. Substitutions may include, for example, conserved amino acids and frequent substitutions based on statistical analyses and evolutionary distance.

Percent identity and homology may be determined, for example, by comparing sequence information using any of a number of computer algorithms known in the art. The GAP program uses the alignment method of Needleman and Wunsch [J. Mol. Biol. 48:443 (1970)], as revised by Smith and Waterman [Adv. Appl. Math. 2:482 (1981)]. Briefly, the GAP program defines similarity as the number of aligned symbols (i.e., nucleotides or amino acids) which are similar, divided by the total number of symbols in the shorter of the two sequences. Default parameters for the GAP program may include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two protein or nucleic acid molecules have amino acid or nucleotide sequences that are at least, for example, 20%, 30%, 40%, or 50%, “identical” can be determined using known computer algorithms such as the “FAST A” program, using for example, the default parameters as in Pearson and Lipman, Proc. Natl. Acad. Sci. USA 85:2444 (1988). The BLAST function of the National Center for Biotechnology Information database may be used to determine identity. By way of example, in a comparison of a test and reference polypeptide, wherein the length of the reference polypeptide is 100 amino acids, a level of 90% or more identity between the polypeptides is indicative of having no more than 10% (i.e., 10 out of 100) amino acids in the test polypeptide differing from that of the reference polypeptide. Similar comparisons may be made between a test and reference polynucleotides. Such differences may be represented as point mutations randomly distributed over the entire length of an amino acid sequence or they may be clustered in one or more locations of varying length up to the maximum allowable, e.g. 10/100 amino acid difference (approximately 90% identity). The BLinK tool (“BLAST Link”) displays the results of precomputed BLAST searches [Altschul et al. (19990) J. Mol. Biol. 215:403-410] that have been done for protein sequences in the Entrez Proteins data domain against the non-redundant (nr) database.

Methods commonly employed to determine identity or homology between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and homology are codified in computer programs. Computer program methods to determine identity and homology between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the terms “identity” and “homology” represent a comparison between two polypeptides or polynucleotides.

Typically, a “test” polypeptide or polynucleotide is compared to a “reference” molecule to determine if there is a significant level of similarity (including identity and/or homology) between the test and reference molecules.

As used herein, the terms at least “X% homology over X% of the protein” or “X% identity over X% of the protein” with reference to a comparison of protein A with protein B refers to protein A having X% homology or X% identity to protein B over X% of the amino acid sequence of protein B. The term “over the protein” means over the length of the amino acid sequence of the protein, but not necessarily over a contigous sequence of amino acids of the protein. Thus, for example, protein B may be 40% homologous to protein A over 60% of protein B; however, the 60% of the amino acids in protein B to which protein A has homology may be located in multiple, separate sequences (e.g., regions or domains) of protein B.

As used herein, protein “homologs” refers to similar proteins encoded by related but different genes either within a species or between species. Protein “orthologs” refers to similar proteins in different species that arose from a common ancestral gene.

As used herein, all assays and procedures, such as hybridization reactions and antibody-antigen reactions, unless otherwise specified, are conducted under conditions recognized by those of skill in the art as standard conditions.

A. Proteins Involved in Modulating Intracellular Calcium

Fluctuations in the level of calcium, including free and bound calcium, in cells provide important biological signals involved in processes such as protein secretion, muscle contraction, cell death and development. The movement of cations, such as calcium, into, within and out of cells thus plays a critical role in the operation and survival of cells. Calcium binding and movement into, out of and within cells can act as a signal that is highly organized in space, frequency and amplitude due in part to localization of the movements and tight regulation of the processes through which calcium movement occurs in cells. One process through which an increase in cytosolic calcium levels can occur is the release of calcium from stores within cells. Another process involves entry of extracellular calcium into cells by movement across the plasma membrane. One mechanism for movement of calcium into cells through the plasma membrane is commonly referred to as store-operated calcium entry.

Altered calcium regulation or calcium dyshomeostasis in cells is associated with a number of diseases and disorders. Altered calcium regulation can be the result of alterations in the elements involved in movement of calcium, and/or in the regulation thereof. It is therefore desirable to identify cellular constituents, such as proteins, that modulate intracellular calcium, including, for example, proteins that provide for and/or regulate the movement of cations, such as calcium, into, within and out of cells, and to identify agents that modulate intracellular calcium.

Identification of molecules, e.g., proteins, involved in modulating intracellular calcium makes possible the elucidation of the molecular and cellular mechanisms underlying modulatory processes such as calcium binding and movement. An example of one such process is store-operated calcium entry, a fundamental and essential process of many cells. The identities of the molecular components that are involved in and/or provide for store-operated calcium entry, and, in particular, the identities of the ion transport proteins or channels that provide for store-operated calcium entry are largely unknown and may differ for different cell types. Identification of such molecules provides molecular targets for the regulation of this specific and critical process in calcium-dependent cellular functions.

Furthermore, identification of molecules inovlved in modulating intracellular calcium assists in the dissection of complex signaling processes and facilitates the elucidation of the element(s) involved in regulation of these processes. Such processes include, but are not limited to, receptor-mediated, store-operated and second messenger-operated cation entry into the cytoplasm or intracellular organelles. Knowledge of the number and structure of such molecules, as well as a comparison of their properties, permits the identification and design of agents that specifically interact with and/or affect or regulate molecules, such as, for example, ion transport proteins, that modulate intracellular calcium. Such agents have many uses. For example, they can be used to assess function and distribution of the proteins. The specific identified proteins may also be used as targets in methods for identifying agents that modulate intracellular calcium levels and candidate therapeutic agents. Furthermore, the identified proteins, as well as nucleic acids encoding the proteins, may be used to modulate intracellular calcium, for example, by recombinant expression of nucleic acid encoding such a protein in a cell or by reducing, altering or interfering with expression of such protein in a cell.

Provided herein are cellular constituents, particularly proteins, involved in modulating intracellular calcium. Proteins identified as being involved in modulating intracellular calcium include, but are not limited to, proteins that have an amino acid sequence that is at least about 35% homologous to a protein encoded by a Drosophila gene over at least about 40% of the encoded protein. The Drosophila gene is one that, when altered in its expression, results in altered intracellular calcium in a Drosophila cell, such as, for example, altered store-operated calcium entry into a Drosophila cell. One such Drosophila cell is an S2 cell. The identified cellular proteins include mammalian proteins, e.g., rodent and human proteins, C. elegans proteins and insect, e.g., Drosophila, proteins. Particular cellular proteins involved in modulating intracellular calcium identified herein are ion transport proteins, including ion transport proteins that do not contain one or both of the following sequences of amino acids: EWKFAR and EXD(E)CR(K)GXYXXYE (wherein “X” represents any amino acid and an amino acid residue in parentheses is an alternative to the residue immediately preceding it).

Proteins identified herein as proteins that modulate intracellular calcium include proteins homologous to a protein encoded by the coding sequence of Drosophila gene CG4536 (Genbank Accession No.

AAF46203; gi7290757; see SEQ ID NO: 1 for the gene coding sequence and SEQ ID NO: 2 for the amino acid sequence) and/or the protein encoded by coding sequence of Drosophila gene CG5842 (Genbank Accession No. AAF49752; gi7294406; see SEQ ID NO: 3 for the gene coding sequence and SEQ ID NO: 4 for the amino acid sequence). As described herein, such proteins have been identified as being involved in, participating in or modulating store-operated calcium entry. Such proteins include, but are not limited to, calcium transport protein 1 (CaT1), epithelial calcium channel (ECaC), CaT2 or CaT-like proteins, olfactory channels, stretch-activated channel proteins, vanilloid receptor-related osmotically activated channel proteins, vanilloid receptor type 1-like proteins, vanilloid receptor subtype 1 proteins, vanilloid receptor 1 proteins, vanilloid receptor-like proteins, capsaicin receptor proteins, OTRPC4 proteins, growth factor-regulated calcium channel proteins, transient receptor potential protein 12 (trp12) and stretch-inhibitable nonselective channel (SIC) proteins. Particular proteins identified herein, including, for example, ion transport proteins, have an amino acid sequence that is at least about 20%, or at least about 25%, or at least about 30%, or at least about 35%, or at least about 40%, or at least about 45%, or at least about 50% or more homologous to the protein encoded by the coding sequence of at least Drosophila gene CG5842 or gene CG4536. Such proteins may be homologous to the specified Drosophila proteins over at least about 30%, or at least about 35%, or at least about 40%, or at least about 50% or more of the protein encoded by the coding sequence of at least Drosophila gene CG5842 or CG4536. Thus, for example, particular proteins identified herein include proteins, such as, for example, ion transport proteins, that have an amino acid sequence that is at least about 35% homologous over at least about 40% of the protein encoded by the coding sequence of at least Drosophila gene CG5842 or CG4536.

Proteins identified herein as being involved in modulating intracellular calcium may participate in one or more of a number of processes that affect intracellular calcium, including, but not limited to, receptor-mediated or second messenger-operated calcium movement, calcium uptake and/or release by intracellular organelles (e.g., endoplasmic reticulum, endosomes and lysosomes) and store-operated calcium entry into cells. A direct participation of a protein in a particular process or pathway not only affects that process but can also indirectly affect one or more other processes or pathways. Thus, for example, a protein that has a direct role in a G-protein-coupled receptor signaling pathway may also indirectly affect store-operated calcium entry into a cell by affecting the release of calcium from the endoplasmic reticulum, which is involved in providing a signal for calcium entry via store-operated ion channels.

Methods are provided herein for screening for and identifying a protein (and/or nucleic acid) involved in modulating intracellular calcium. Particular methods provided herein are for screening for candidate proteins, such as ion transport proteins, that are involved in, participate in, modulate and/or provide for store-operated calcium entry into cells and for identifying such proteins.

Also provided herein are methods for identifying additional elements (e.g., proteins and other cellular or cell-associated molecules) involved in modulating intracellular calcium (and, in particular, store-operated calcium entry), identifying agents that modulate intracellular calcium (and, in particular, store-operated calcium entry) and methods for modulating intracellular calcium (and, in particular, store-operated calcium entry) and for treating diseases and disorders. Such methods can utilize or target the proteins identified herein as being involved in modulating intracellular calcium, such as, for example, by modulating or by participating in stored-operated calcium entry.

B. Cellular Signaling

All living cells sense and respond to their environment by a set of mechanisms termed cell signaling. These mechanisms are part of a complex system of communication that govern basic cellular activities and coordinate the actions of cells. Living cells must respond appropriately to their environment, whether they are free-living in the soil or part of a tissue. Cell communication is necessary for the existence of multicellular organisms. The ability of cells to perceive and correctly respond to their microenvironment is the basis of development, tissue repair, and immunity as well as normal tissue homeostasis.

Cells, through a class of proteins known as receptors, receive information from their environment. The information is then processed through signaling pathways and decoded in the nucleus and other areas of the cell. The spatial and temporal dynamics of both receptors and the components of the signaling pathways are important in the transfer of the message from the extracellular to the intracellular environment. The components of the signaling pathway, their location either inside or on the surface of the cell, the role of each component in the transduction of the signal, and interactions among the components of the system in order to transduce the signal are required to elucidate the cause, mechanism and effect of the signal transduction on the cell. Molecules that can serve as components of a signaling pathway include, but are not limited to, an organic compound, inorganic compound, metal complex, receptor, enzyme, antibody, protein, nucleic acid, peptide nucleic acid, DNA, RNA, polynucleotide, oligonucleotide, oligosaccharide, lipid, lipoprotein, amino acid, peptide, polypeptide, peptidomimetic, carbohydrate, cofactor, drug, prodrug, lectin, sugar, glycoprotein, hormone, steroid, biomolecule, macromolecule, biopolymer, polymer, sub-cellular structure, sub-cellular compartment or any combination, portion, salt, or derivative thereof, a virus, such as a viral vector or viral capsid with or without packaged nucleic acid, phage, including a phage vector or phage capsid, with or without encapsulated nucleotide acid, a cell, including eukaryotic and prokaryotic cells or fragments thereof, a liposome or micellar agent or other packaging particle, and other such biological materials. The interactions among the components of the signaling system include, but are not limited to, protein:protein, protein:nucleic acid, nucleic acid:nucleic acid, protein:lipid, lipid:lipid, protein:small molecule, receptor:signal, antibody:antigen, peptide nucleic acid:nucleic acid, and small molecule:nucleic acid.

A key component in many signaling pathways is calcium. Intracellular calcium concentration is tightly regulated, and numerous cellular constitutents, e.g., proteins, and processes are calcium sensitive. Thus, many signal transduction mechanisms involve transient calcium flux across the plasma membrane and membranes of intracellular organelles.

Errors in cellular information processing contribute to numerous diseases, including, but not limited to, cancer, autoimmunity, and diabetes. In order to effectively treat these diseases, knowledge of the mechanism by which the signal is transduced is required. Identifying the components of the signaling pathway and the message that it transmits allows for the alteration, blocking or amplification of the message and possible prevention of the undesired cellular response or activation of desired cellular response. This information can also be used in other ways such as, but not limited to, controlling the behavior of individual cells and permitting the creation of artificial tissues.

C. Calcium Homeostasis

Cellular calcium homeostasis is a result of the summation of regulatory systems involved in the control of intracellular calcium levels and movements. Cellular calcium homeostasis is achieved, at least in part, by calcium binding and by movement of calcium into and out of the cell across the plasma membrane and within the cell by movement of calcium across membranes of intracellular organelles including, for example, the endoplasmic reticulum, sarcoplasmic reticulum, mitochondria and endocytic organelles including endosomes and lysosomes.

Movement of calcium across cellular membranes is carried out by specialized proteins. For example, calcium from the extracellular space can enter the cell through various calcium channels and a sodium/calcium exchanger and is actively extruded from the cell by calcium pumps and sodium/calcium exchangers. Calcium can also be released from internal stores through inositol trisphosphate or ryanodine receptors and can be taken up by these organelles by means of calcium pumps.

Endocytosis provides another process by which cells can take up calcium from the extracellular medium through endosomes. In addition, some cells, e.g., exocrine cells, can release calcium via exocytosis.

1. Basal or Resting Cytosolic Calcium Levels

Cytosolic calcium concentration is tightly regulated with resting levels usually estimated at approximately 0.1 μM in mammalian cells, whereas the extracellular calcium concentration is typically about 2 mM. This tight regulation facilitates transduction of signals into and within cells through transient calcium flux across the plasma membrane and membranes of intracellular organelles. As a result, disturbance of resting cytosolic calcium levels can effect transmission of such signals and give rise to defects in a number of cellular processes. For example, cell proliferation involves a prolonged calcium signaling sequence. Other cellular processes, including but not limited to, secretion, fertilization and learning, involve calcium signaling.

2. Store-Operated Calcium Entry

One mechanism for movement of calcium into cells through the plasma membrane is commonly referred to as store-operated calcium entry. Reduced calcium concentration in intracellular calcium stores such as the endoplasmic reticulum resulting from release of calcium therefrom provides a signal for influx of calcium from the extracellular medium into the cell. This influx of calcium, which produces a sustained “plateau” elevation of cytosolic calcium concentration, generally does not rely on voltage-gated plasma membrane channels and does not involve activation of calcium channels by calcium. This calcium influx mechanism has been referred to as capacitative calcium entry (CCE), calcium-release activated, store-operated or depletion-operated calcium entry. Store-operated calcium entry can be recorded as an ionic current with distinctive properties. In some instances, this current is referred to as I_{SOC} (store-operated current) or I_{CRAC} (calcium release-activated current).

a. Regulation of Store-Operated Calcium Entry by Intracellular Calcium Stores

Store-operated calcium entry is regulated by the level of calcium within an intracellular calcium store. Intracellular calcium stores can be characterized by sensitivity to agents, which can be physiological or pharmacological, that activate release of calcium from the stores or inhibit uptake of calcium into the stores. Different cells have been studied in characterization of intracellular calcium stores, and stores have been characterized as sensitive to various agents, including, but not limited to, IP₃ and compounds that effect the IP₃ receptor, thapsigarin, ionomycin and/or cyclic ADP-ribose (cADPR) [see, e.g., Berridge (1993) Nature 361:315-325; Churchill and Louis (1999) Am. J. Physiol. 276:C426-C434; Dargie et al. (1990) Cell Regul. 1:279-290; Gerasimenko et al. (1996) Cell 84:473-480; Gromoda et al. (1995) FEBS Lett. 360:303-306; Guse et al. (1999) Nature 398:70-73].

The identities and/or cellular locations of calcium stores can be determined, for example, by isolation and characterization of organelles or imaging of cells using calcium-sensitive indicators which localize in storage organelles. Mag-fura 2, an example of one such indicator, is a UV light-excitable, ratiometric, low-affinity fluorescent calcium indicator. The moderate calcium affinity of mag-fura-2 and the tendency of its acetoxymethyl (AM) ester to accumulate in subcellular compartments makes this indicator particularly useful in monitoring of calcium stores [see, e.g., Hofer and Machen (1993) Proc. Natl. Acad. Sci. U.S.A. 90:2598-2602; Hofer et al. (1998) EMBO J. 17:1986-1995; Hofer et al. (1998) J. Cell Biol. 140:325-334; Churchill and Louis (1999) Am. J. Physiol. 276:C426-C434]. Intracellular calcium stores include the endoplasmic reticulum and sarcoplasmic reticulum, which are sensitive to IP₃ or caffeine/ryanodine and thapsigarin/cyclopiazonic acid (CPA) [see, e.g., Pozzan et al. (1994) Physiol. Rev. 74:595-637; Meldolesi and Pozzan (1998) J. Cell Biol. 142:1395-1398; Meldolesi and Pozzan (1998) Trends Biochem. Sci. 23:10-14; Golovina and Blaustein (2000) Glia 31:15-28], and isolated zymogen granules and the envelope of isolated nuclei, which are sensitive to cADPR and IP₃ [see, e.g., Gerasimenko et al. (1996) Cell 84:473-480; Gerasimenko et al. (1995) Cell 80:439-444].

Basal free calcium concentrations in calcium stores can be orders of magnitude, e.g., 10³-fold, greater than the free calcium concentration in the cytosol. For example, the basal free calcium concentration measured in the endoplasmic reticulum of HEK293 cells ranges between about 200-700 μM, with an average of about 500 μM, whereas the basal free calcium concentration in the cytosol is about 50 nM [Yu and Hinkle (2000) J. Biol. Chem. 275:23648-23653].

Free calcium concentrations in the endoplasmic reticulum can be measured in a variety of ways such as, for example, using various calcium-sensitive indicators [see, e.g., Yu and Hinkle (2000) J. Biol. Chem. 275:23648-23653] including mag-fura 2 [see, e.g., Hofer and Schulz (1996) Cell Calcium 20:235-242], endoplasmic reticulum-targeted aequorin [see, e.g., Montero et al. (1995) EMBO J. 14:5467-5475] and endoplasmic reticulum-targeted “cameleons” [i.e., fluorescent calcium indicators based on fluorescence resonance energy transfer between two modified green fluorescent proteins (GFPs) contained in a protein with calmodulin and a calmodulin-binding peptide; see, e.g., Miyawaki et al. (1997) Nature 388:882-887 and Yu and Hinkle (2000) J. Biol. Chem. 275:23648-23653].

Accumulation of calcium within endoplasmic reticulum and sarcoplasmic reticulum (SR; a specialized version of the endoplasmic reticulum in striated muscle) storage organelles is achieved through sarcoplasmic-endoplasmic reticulum calcium ATPases (SERCAs), commonly referred to as calcium pumps. During signaling (i.e., when endoplasmic reticulum channels are activated to provide for calcium release from the endoplasmic reticulum into the cytoplasm), endoplasmic reticulum calcium is replenished by the SERCA pump with cytoplasmic calcium that has entered the cell from the extracellular medium [Yu and Hinkle (2000) J. Biol. Chem. 275:23648-23653; Hofer et al. (1998) EMBO J. 17:1986-1995].

Calcium release channels associated with IP₃ and ryanodine receptors provide for controlled release of calcium from endoplasmic and sarcoplasmic reticulum into the cytoplasm resulting in transient increases in cytoplasmic calcium concentration. IP₃ receptor-mediated calcium release is triggered by IP₃ formed in the break down of plasma membrane phosphoinositides through the action of phospholipase C activated by binding of an agonist to a plasma membrane G protein-coupled receptor. Ryanodine receptor-mediated calcium release is triggered by an increase in cytoplasmic calcium and is referred to as calcium-induced calcium release (CICR). The activity of ryanodine receptors (which have affinity for ryanodine and caffeine) may also be regulated by cyclic ADP-ribose.

Thus, the calcium levels in the stores, and in the cytoplasm, fluctuate. For example, ER free calcium can decrease from a range of about 60-400 μM to about 1-50 μM when HeLa cells are treated with histamine, an agonist of PLC-linked histamine receptors [Miyawaki et al. (1997) Nature 388:882-887]. Store-operated calcium entry is activated as the free calcium concentration of the intracellular stores is reduced. Depletion of store calcium, as well as a concomitant increase in cytosolic calcium concentration, can thus regulate store-operated calcium entry into cells.

b. Store-Operated Ionic Currents

Electrophysiological analysis of store-operated or calcium release-activated currents reveals distinct biophysical properties [see, e.g., Parekh and Penner (1997) Physiol. Rev. 77:901-930] of these currents. For example, the current can be activated by depletion of intracellular calcium stores, and can be selective for divalent cations, such as calcium, over monovalent ions, can be influenced by changes in cytoplasmic calcium levels, and can show altered selectivity and conductivity in the presence of low extracellular concentrations of divalent cations.

3. Receptor-Mediated and Second Messenger-Operated Cation Movement

Receptor-mediated cation channels are gated in response to ligand binding to a membrane receptor distinct from the channel protein itself. Some receptor-mediated cation channels are activated downstream of tyrosine kinases and others via G protein signaling cascades. Receptor-mediated channels are expressed in a number of both excitable and nonexcitable cells, including smooth muscle, mast cells, epidermis and renal mesangial cells.

One way in which receptor-mediated cation channels are regulated is through second messengers induced in response to ligand-binding to a membrane receptor. Such cation channels are referred to as second messenger-operated channels. For example, cyclic nucleotides generated by adenylyl and guanylyl cyclases can directly activate cation-permeable channels. Such cyclic nucleotide-gated channels are predominantly expressed in sensory tissues, for example the retina and in olfactory/gustatory epithelia. Calcium is another second messenger that can mediate ion channel function. Examples of calcium-mediated channels include calcium-activated potassium and chloride channels as well as cation channels in neutrophils, smooth muscle and mast cells.

Inositol phosphates generated upon activation of phospholipase C (PLC) can also act as second messengers that activate certain channels. For example, channels responsive to inositol-1,4,5-triphosphate (IP₃) include the intracellular IP₃ receptor of the endoplasmic reticulum as well as plasma membrane channels such as those expressed in T-lymphocytes, mast cells and epidermal cells. The intracellular IP₃ receptor functions as a ligand-gated ion channel that permits passage of calcium upon binding of IP₃ released through hydolysis of membrane phospholipids by activated phospholipase C (PLC). PLC can be activated through agonist binding to a surface membrane G protein-coupled receptor. Activation of the IP₃ receptor results in the release of calcium stored in the endoplasmic reticulum into the cytoplasm which produces a transient “peak” increase in cytosolic calcium concentration. In addition, although no cation channels have been identified in components of the endocytic pathway, e.g., endosomes and lysosomes, IP₃-dependent agonists appear to be associated with calcium release from lysosomes in MDCK cells (see Haller et al. (1996) Biochem. J. 319:909-912).

Lipids and polyunsaturated fatty acids (PUFAs) may also act as second messengers for the activation of ion channels. For example, arachidonic acid and its metabolites, as well as linolenic acid, can activate receptor-mediated ion channels. In addition, phospholipids, such as lysophospholipids (e.g.,lysophosphatidic acid (LPA), lysophosphatidylcholine (LPC), sphingosylphosphoryl choline (SPC) and sphingosine 1-phosphate (SIP)) can be ligands for plasma membrane receptors such as G-protein-coupled receptors (GPCRs) involved in a second messenger cascade process in cells (see, e.g., HIa et al. (2001) Science 294:1875). PLC generates not only IP₃ but also diacylglycerol (DAG) which is a potential precursor for polyunsaturated fatty acids.

PUFAs can be released from DAG by the action of DAG lipase.

4. Calcium Uptake and Release by Endosomes and Lysosomes

Endocytosis is a process whereby contents of the cell plasma membrane and extracellular medium are transported into the interior of the cell. Not only does endocytosis serve “house-keeping” functions of a cell, it plays crucial roles in cell signaling, development, and the regulation of varied biological processes including, for example, synaptogenesis, neural plasticity, generation of morphogen gradients and programmed cell death. Endocytosis may have both negative and positive influences on signaling. For example, endocytosis can regulate the number of receptors on the plasma membrane. In addition, endocytosis plays a positive role in signaling mediated by the Notch signaling pathway which acts to determine cell-type specificity during development.

The endocytic process involves several components, including endosomes and lysosomes which are intracellular compartments along the endocytic pathway. Endosomes are morphologically heterogeneous and constitute a pleiomorphic smooth membrane system of tubular and vesicular elements. The vesicular elements contain intra-organelle vesicles and are described as multivesicular bodies. Endocytosed macromolecules are delivered first to early endosomes and then to late endosomes. Early endosomes are tubular with varicosities and many are located peripherally within the cell close to the plasma membrane. Late endosomes are more spherical and have the appearance of multivesicular bodies. They are mostly juxtanuclear being concentrated near the microtubule organizing center. Early and late endosomes are characterized by different lumenal pHs, different protein compositions and association with different small GTPases of the Rab family. The early endosome is the major sorting compartment of the endocytic pathway where many ligands dissociate from their receptors and from which many of the receptors recycle to the cell surface.

Lysosomes are membrane-bound organelles containing hydrolytic enzymes and are regarded as the terminal degradation compartment of the endocytic pathway. Lysosomes also play an important role in phagocytosis, autophagy, crinophagy and proteolysis of some cytosolic proteins that are transported across the lysosomal membrane. In many cell types, lysosomes secrete their contents after fusion with the plasma membrane. The limiting membrane of lysosomes contains a set of highly glycosylated lysosomal-associated membrane proteins (LAMPs). Additional lysosomal membrane proteins mediate transport of ions, amino acids, and other solutes across the lysosomal membrane.

In mammalian cells, the organelles of the late endocytic pathway interact with each other and are in dynamic equilibrium. Content mixing and/or exchange of membrane proteins occurs between late endosomes, lysosomes and between late endosomes and lysosomes. Delivery of endocytosed macromolecules to lysosomes occurs by content mixing between late endosomes and lysosomes as a result of transient as well as direct fusion which can form hybrid organelles. Cell-free heterotypic fusion of mammalian late endosomes and lysosomes requires calcium which may mediate its effects via calmodulin. The calcium is derived and released from the endocytic organelle lumen and is required in a late step in fusion. Although the calcium release pathway has not yet been elucidated, docking of endocytic organelles is thought to trigger release of endocytosed calcium from the lumen of endocytic organelles into the cytoplasm. This calcium release is believed to mediate membrane fusion events at several stages on the endocytic pathway. Lysosomes contain a mobilizable calcium pool. Furthermore, although no cation transport molecule has been identified in endosomes or lysosomes, both endosomal and lysosomal membranes provide for calcium transport. Thus, uptake and release of calcium from endosomes and lysosomes can impact intracellular and cytosolic calcium levels.

D. Types of Molecules that can be Involved in Modulating Intracellular Calcium

Molecules that can be involved in modulating intracellular calcium include, but are not limited to, calcium-binding proteins, ion transport proteins that are involved in providing for movement of cations, such as calcium, into, out of or within cells, and proteins that regulate ion transport proteins, receptors or calcium-binding proteins. A molecule involved in modulating intracellular calcium can do so at any level and in connection with any of a number of processes within a cell. For example, a molecule involved in modulating intracellular calcium may participate in the maintenance of resting cytosolic calcium levels, store-operated calcium entry into cells, receptor-mediated calcium movement, second messenger-operated calcium movement, calcium influx into or efflux from a cell, and/or calcium uptake into or release from intracellular compartments, including, for example, the endoplasmic reticulum, endosomes and lysosomes. A molecule involved in modulating intracellular calcium may function alone (e.g., as a single unit or as a homo-multimer of two or more of the proteins) or in combination with other proteins (e.g., in a heteromeric configuration), and may be involved in regulating proteins that bind and/or transport calcium or receptors, particularly receptors involved in receptor-mediated cation movement or cell signaling.

1. Ion Transport Proteins

Ion transport proteins are proteins involved in providing for the transport of ions into, within, or out of cells. Ion transport proteins involved in modulating intracellular calcium are involved in providing for the transport of calcium. Such ion transport proteins may be relatively specific for calcium ion transport.

a. Structural Features of Ion Transport Proteins

An ion transport protein may function to transport cations in a number of ways. For example, the proteins may form a pore or channel for the transport of cations through a membrane. The proteins may instead provide for a translocation of cations through ion binding and release processes as is characteristic of a transporter. Ion transport proteins may function to transport ions as a single unit or may be one unit of a multi-component structure that transports ions. A multi-component structure may be a homo-multimer of two or more of the same proteins or a hetero-multimer of two or more different proteins.

Ion transport proteins involved in providing for movement of ions via a channel-like or pore structure typically contain one or multiple regions that have characteristics of transmembrane domains, which may possibly participate in channel formation. Transmembrane domains tend to include a sequence of amino acids, typically of about 10 to about 30 or more amino acids, about 15 to about 30 or more amino acids, about 20 to about 30 or more amino acids, or about 20 to about 25 amino acids, of high hydrophobicity. The hydrophobicity scale is defined from the transfer free energy of amino acids between organic solvents and water, and statistics on the distribution of residues in proteins. Hydropathy plots of the hydrophobicity of adjacent amino acid residues averaged over a moving window of suitable length are commonly used to assess proteins for such sequences of amino acids [see, e.g., Kyte and Doolittle (1982) J. Mol. Biol. 157:105-134; Argos et al. (1982) Eur. J. Biochem. 128:565-575 and Engelman et al. (1984) Ann. Rev. Biophys. Biophys. Chem. 15:321-353]. Although transmembrane domains may be in an α-helical and/or β-sheet conformation, the transmembrane domain structure of most major families of ion channel proteins appears to be α-helical.

Pore-lining segments of ion transport protein channel transmembrane domains, such as regions lining aqueous channels, may have a partly hydrophilic face and appear as amphipathic segments. Amphipathic segments may be identified based on the hydrophobic moment [see, e.g., Eisenberg et al. (1984) J. Mol. Biol. 179:125-142; and Finer-Moore and Stroud (1984) Proc. Natl. Acad. U.S.A. 81:155-159].

The number of transmembrane domains contained in an ion transport protein that includes a channel-like structure can vary. For example, there can be at least one transmembrane domain contained in a protein involved in providing for ion transport. Thus, for example, there can be one, two, three, four, five, six or more transmembrane domains contained in a protein involved in providing for ion transport.Typically, there are at least about 1 to about 25 or more transmembrane domains, about 2 to about 25 or more transmembrane domains, about 4 to about 25 or more transmembrane domains, about 6 to about 25 or more transmembrane domains, about 8 to about 25 or more transmembrane domains, or about 1 or 2 to about 8 transmembrane domains or about 6 or more transmembrane domains. For example, an ion transport protein that includes a channel-like structure and is capable of providing for the movement of cations through a membrane may contain at least one, or at least two, or at least three or at least 4 or more groups of six transmembrane helices.

b. Calcium Transport

Ion transport proteins that modulate intracellular calcium are involved in the transport of calcium. Calcium transport may be assessed in a variety ways. For example, cells expressing an ion transport protein may be evaluated for uptake of labeled calcium, such as ⁴⁵Ca²⁺, into the cells. RNA coding for an ion transport protein may also be introduced into a cell, e.g., Xenopus laevis oocytes, which may be evaluated for ⁴⁵Ca²⁺ uptake.

Calcium transport properties of an ion transport protein may also be assessed using calcium indicator-based assays of the intracellular calcium levels of cells expressing the protein. Such assays utilize calcium-sensitive indicators which facilitate detection of transient alterations in intracellular calcium levels. These indicators provide a detectable signal, e.g., fluorescence or bioluminescence, upon binding of calcium and therefore can be correlated to calcium levels in cells. Methods of measuring intracellular calcium using calcium indicators are well known in the art [see, e.g., Takahashi et al. (1999) Physiol. Rev. 79:1089-1125].

Electrophysiological analysis of cells expressing an ion transport protein also can be used to assess calcium transport by a transport protein. For example, whole-cell, patch-clamp, voltage clamp and single-channel recording methods may be used to detect and measure calcium or other cation currents across membranes of cells to which calcium or another cation has been applied. A variety of cells may be used for such electrophysiological analysis, including, but not limited to, X. laevis oocytes into which RNA encoding an ion transport protein has been injected.

Ion transport protein(s) involved in calcium regulation may be more permeable to calcium than to monovalent ions under physiological conditions. Thus, such types of proteins are able to flux calcium through the membrane more readily than monovalent ions. For example, the permeability of an ion transport protein to calcium may be more than about 1.5-fold, more than about 2-fold, more than about 3-fold, more than about 4-fold, more than about 5-fold, more than about 6-fold, more than about 7-fold, more than about 8-fold, more than about 9-fold or more than about 10-fold greater than the permeability to monovalent ions. Ion transport proteins may be selective for calcium under physiological conditions.

2. Modulatory or Regulatory Molecules

In addition to ion transport proteins, there may be cellular molecules that are involved in modulating intracellular calcium but that are not directly involved in providing for the transport of ions into or out of the cytosol as would, for example, a channel protein. Such molecules, which can be referred to as modulatory or regulatory molecules (i.e., modulators or regulators), encompass a diverse array of structures and include receptors, enzymes and calcium-binding proteins. Modulator or regulator molecules can, for example, function in processing a signal in a cell (e.g., signaling pathway components) or regulate the activity of an enzyme or ion channel.

For example, some calcium-binding proteins can be involved in modulating intracellular calcium. Included among the calcium-binding proteins are proteins that contain a calcium binding motif referred to as an EF-hand [see, e.g., Kretsinger (1997) Nat. Struct. Biol. 4:514-516; Ikura (1996) Trends Biochem. Sci. 21:14-17; Kawasaki et al. (1995) Protein Profile 2:297-490]. Typically, an EF-hand motif contains a loop of about 12 amino acid residues flanked on either side by an alpha helix of about 12 amino acid residues. Position 12 of the loop typically contains a Glu or Asp. The helix-loop-helix motif can be repeated from about 2 to about 12 times. The motifs may coordinate calcium to side-chain oxygens of invariant residues occupying positions 1, 3, 5 and 12 of the loop, and to a carbonyl oxygen of a less conserved residue at position 7. EF hand-containing proteins may undergo a conformational change upon binding calcium, and thus some of these proteins can pass signaling information on to targets to which such proteins can bind. EF hand proteins can function in a number of ways. For example, they can function as a separate subunit of a single protein (e.g., enzyme), as a subunit that reversibly associates with different proteins (e.g., calmodulin) or as an integral portion of the sequence of an enzyme (e.g., calpain).

An example of an EF hand protein is calmodulin (CaM). Calmodulin interacts with numerous diverse proteins, including, for example, CaM-dependent serine/threonine kinases, CaM kinase II, CaM kinase kinase and myosin light chain kinase, calcineurin, CaM-calcium-activated potassium channel and anthrax adenylyl cyclase. The roles of CaM binding to such target proteins include, for example, release of autoinhibitory domains of kinases, CaM tethering and calcium-dependent inactivation of the target protein, active site remodeling of adenylyl cyclase, dimerization and activation of channel proteins. CaM recruitment motifs of CaM-binding domains of CaM-dependent proteins have been predicted (see, e.g., Rhoads and Friedberg (1997) FASEB J. 11:331-340; Hoeflich and Ikura (2002) Cell 108:739-742 and http://calcium.uhnres.utoronto.ca). An example of a CaM-binding motif is an “IQ motif” having the following consensus sequence which can be present in tandem repeats: IQXXXRGXXXR.

Neuronal calcium sensor (NCS) proteins also contain EF hand motifs. These proteins are expressed predominantly or solely in retinal photoreceptors or neurons. Five subfamilies of NCS proteins have been described: two expressed in retinal photoreceptors (recoverins, which inhibit rhodopsin kinase, and the guanylate cyclase-activating proteins (GCAPs)) and three expressed in central neurons and neuroendocrine cells (frequenins, visinin-like proteins and the Kv channel-interacting proteins) (see, e.g., Burgoyne and Weiss (2002) Biochem. J. 353:1-12). The latter three subfamilies may regulate neurotransmitter release, polyphosphoinositide biosynthesis, cyclic nucleotide metabolism and the activity of type A potassium channels. The NCS proteins undergo conformational changes upon binding calcium and have thus been referred to as calcium sensors or switches. Most NCS proteins are N-terminally myristoylated, and, after binding of calcium, the myristoyl residue and hydrophobic portions of the sequence are exposed favoring interaction with membranes or target proteins.

Another family of EF hand-containing proteins is the calpain family of intracellular cysteine proteases. These proteins modulate biological activities of their substrates by limited proteolysis. The protease core region of the μ isoform of calpain appears to also contain two non-EF hand calcium-binding sites. Binding of calcium at these sites aligns the active site cleft and converts the core into an active enzyme with calpain-like specificity (see, e.g., Moldoveanu et al. (2002) Cell 108:649-660). Thus, there are at least three different types of calcium-binding sites in calpains: EF-hand, C2-like domain and protease domain sites.

E. Methods of Screening for and Identifying Proteins Involved in Modulating Intracellular Calcium

Methods are provided herein for screening for and identifying proteins, and/or nucleic acids that encode proteins, involved in modulating intracellular calcium. Such proteins include, but are not limited to, calcium-binding proteins, ion transport proteins that are involved in providing for movement of cations, such as calcium, into, out of or within cells, receptors and enzymes as well as proteins that regulate ion transport proteins, receptors, enzymes or calcium-binding proteins. In a particular embodiment, methods provided herein are for screening for and identifying proteins, and/or nucleic acids that encode proteins, that are involved in, participate in, regulate and/or provide for store-operated calcium entry into cells.

Intracellular calcium-modulating protein identification methods provided herein involve reduction, alteration or elimination of the expression of one or more genes in a cell and assessing intracellular and/or cytosolic calcium levels and/or calcium movement into, out of or within the cell following reduction, alteration or elimination of the expression of one or more genes. In particular embodiments of the methods, store-operated calcium entry, resting cytosolic calcium levels and/or intracellular organelle (e.g., endoplasmic reticulum) calcium levels, uptake or release is (are) assessed following reduction, alteration or elimination of the expression of one or more genes. The methods are useful in identifying new proteins and/or nucleic acids encoding new proteins that were previously unknown and that are involved in modulating intracellular calcium. The methods are also useful in identifying known proteins that have not been associated with intracellular calcium modulation, or that have not been associated with a particular aspect of intracellular calcium modulation (such as store-operated calcium entry or uptake or release of calcium from intracellular organelles), as intracellular calcium-modulating proteins or as being involved in, participating in or providing for store-operated calcium entry, maintenance of resting cytosolic calcium levels or calcium uptake, release or storage by intracellular organelles.

1. Cells for use in Methods for Screening for and Identifying Proteins Involved in Modulating Intracellular Calcium

a. General Features of Cells

The methods for identifying a protein (and/or nucleic acid encoding a protein) involved in modulating intracellular calcium involve reduction, alteration or elimination of the expression of one or more genes in a cell and the assessment of intracellular and/or cytosolic calcium levels and/or calcium movement into, within or out of the cells. Although any cell may be used in the methods, cells that are particularly suitable are those that exhibit one or more calcium transport processes and/or those in which calcium levels and/or movement may readily be assessed.

Another feature of cells that are particularly suitable for use in the screening and identification methods is amenability to gene expression alteration. A number of techniques for altering gene expression in cells are known in the art and described herein. The relative ease with which these techniques may be applied to a cell to effect reduction, alteration or elimination of one or more genes in the cell is a consideration in selection of cells for use in the methods provided herein. Amenability to gene expression alteration and analysis of ion flux particularly may be considerations, for example, when a number of genes will be screened.

In particular embodiments of the methods, the cells used in the method are from an organism whose genome has been extensively sequenced. Included among such organisms are non-mammalian eukaryotic organisms, such as Drosophila (in particular D. melanogaster), Caenorhabditis (in particular, C. elegans), Anopheles (in particular, A. gambiae), pufferfish (in particular, Fugu rubripes) and zebrafish (in particular, Danio rerio) and mammals, such as, for example, rodents and humans. The design of elements such as nucleic acids used in altering gene expression (e.g., in antisense RNA, RNA interference and gene knock-out or knock-in methods) in such cells is greatly facilitated by the availability of genomic sequence information.

Cell type may also be a consideration when it is desired to identify an intracellular calcium-modulating protein of a particular cell type or that is involved in a particular aspect of calcium modulation. For example, for identification of a protein involved in store-operated calcium entry, a cell used in the method could be one that exhibits store-operated calcium entry, and a particular cell could be a central nervous system (CNS) cell.

b. Cells that Exhibit Store-Operated Calcium Entry

In a particular method for identifying a protein (and/or nucleic acid encoding a protein) that modulates intracellular calcium, the cell used is one that exhibits store-operated calcium entry. In such methods, store-operated calcium entry can be assessed following reduction, alteration or elimination of the expression of one or more genes in the cell to identify proteins (and/or nucleic acids encoding proteins) that are involved in, participate in, regulate and/or provide for store-operated calcium entry. Cells that exhibit store-operated calcium entry can readily be identified. Any method of determining the occurrence of calcium entry into a cell that distinguishes store-operated calcium entry from other types of calcium influx (e.g., entry through voltage-gated calcium channels) can be used to determine if a cell exhibits store-operated calcium entry. Cells that exhibit store-operated calcium entry include insect cells, e.g., Drosophila cells (e.g., Schneider 2 or S2 cells), human embryonic kidney (HEK) cells, neuronal or nervous system cells, e.g., SHSY5Y neuroblastoma cells and PC12 cells, rat basophilic leukemia (RBL) cells, and immune system cells, e.g., lymphocytes such as T lymphocytes, including Jurkat cells.

For example, in one method for detecting store-operated transport of calcium across the plasma membrane, cells may be treated to reduce the calcium levels of intracellular calcium stores and then analyzed for evidence of calcium influx in response thereto. Techniques for reducing calcium levels of intracellular stores and for analyzing cells for evidence of calcium influx are known in the art and described herein.

In other methods, diffusable signals may be used to activate store-operated transport of calcium across the plasma membrane in methods of detecting the same. One such signal is referred to as calcium influx factor (CIF) [see, e.g., Randriamampita and Tsien (1993) Nature 364:809-814; Parekh et al. (1993) Nature 364:814-818; Csutora et al. (1999) Proc. Natl. Acad. Sci. U.S.A. 96:121-126], which may be a small (˜<500D) phosphate-containing anion. A CIF activity from thapsigargin-treated Jurkat cells, as well as a similar activity from calcium pump-deficient yeast, can activate calcium influx in Xenopus oocytes and in Jurkat cells. When included in the patch pipette during whole-cell patch clamp of Jurkat cells, the extracts activate an inward current resembling I_{CRAC}.

In other methods, electrophysiological analysis of currents across a cell-detached plasma membrane patch of a cell may be used to detect currents associated with or characteristic of store-operated transport of calcium.

(1) Reduction of Calcium Levels of Intracellular Stores

A variety of cell treatments may be used to reduce calcium levels in intracellular calcium stores. Generally, the treatments can be viewed as either an active direct reduction in calcium levels, such as by removal of free calcium from the stores (i.e., “active” depletion), or a passive reduction in calcium levels, such as by leak of calcium from the stores either by a reduction in the availability of free calcium for filling or replenishing the stores or by preventing filling or replenishing of stores (i.e., “passive” depletion).

(a) Passive Depletion

One method of reducing the availability of calcium for the internal calcium stores is to decrease the calcium concentration of the extracellular medium and/or cytoplasm. Calcium concentrations of these fluids can be decreased using cation, and particularly calcium, chelators including, but not limited to EGTA and 1,2-bis (2-amino-phenoxy)ethane-N,N,N,N′-tetraacetic acid (BAPTA). For example, cells may be equilibrated in 10 mM external calcium with strong buffering of cytosolic calcium, for example, through dialysis with 10 mM EGTA. Alternatively, reduction of external calcium may also deplete intracellular calcium stores in many types of cells. For example, cells may be incubated in nominally free calcium solution, e.g., 10 μM external calcium, or essentially calcium-free solution, e.g., ˜1 nM external calcium with strongly (e.g., 10 mM EGTA) buffered cytosolic calcium.

Reduction of calcium in intracellular calcium stores can also be accomplished by application of an agent that blocks endoplasmic reticulum calcium ATPase pumps (SERCAs), thereby reducing or preventing refilling of the endoplasmic reticulum with calcium and providing for leak of calcium from the ER into the cytoplasm resulting in a reduction of ER free calcium concentration. For example, ER free calcium concentration may decrease from about 500 μM to about 50-100 μM as has been observed in HEK293 cells [Yu and Hinkle (2000) J. Biol. Chem. 275:23648-23653. Such agents include, but are not limited to, thapsigargin, cyclopiazonic acid (CPA) and di-tert-butyl-hydroquinone (tBHQ).

(b) Active Depletion

Active reduction of calcium levels in intracellular calcium stores can be experimentally implemented in a number of ways. For example, exposure of endoplasmic reticulum inositol-1,4,5-triphosphate (IP₃) receptors to IP₃ or derivatives or analogs thereof provides for release of calcium from this calcium store and serves to reduce calcium levels therein. IP₃ or derivatives or analogs thereof can be provided to the endoplasmic reticulum through direct intracellular application, through application to the plasma membrane (using membrane-permeable derivatives of IP₃) or by contacting cells with an agent that activates the phosphoinositide cascade to generate IP₃. Such activating agents include agonists of plasma membrane receptors linked to activation of PLC and agents that activate PLC downstream of the plasma membrane in the signalling cascade. Examples of G protein-coupled receptor agonists include histamine, muscarine, carbachol, substance P and glutamate. When activation of the phosphoinositide cascade is used to actively deplete store calcium, the cell used in the method can be one that expresses, either endogenously or recombinantly, a membrane receptor linked to phospholipase C activation, such as, for example, muscarinic receptors, Bradykinin receptors. Additionally, inhibition of catabolic enzymes involved in degradation of IP₃ can serve to provide IP₃ for interaction with its receptor.

A membrane-permeant cation chelator that can chelate calcium within internal stores may also be used to reduce free calcium levels of the stores. One such chelator is N,N,N′,N′-tetrakis (2-pyridylmethyl)ethylene diamine (TPEN), which, in its uncomplexed form, diffuses across cell membranes [see, e.g., Hofer et al. (1998) J. Cell Biol. 140:325-334]. Because this multivalent cation chelator has a low affinity for calcium, it should not significantly influence calcium levels in the cytoplasm or other cell compartments where the steady-state calcium concentration is in the nanomolar or low micromolar range. In cell compartments where the calcium concentration is comparable to its K_{d}, such as, for example, the endoplasmic reticulum, TPEN should bind calcium to rapidly reduce free calcium levels. Removal of TPEN from the cell medium should provide for increases in free calcium levels in such cell compartments due to rapid unbinding of the chelator from calcium ions and diffusion of the free form of TPEN from the compartment. Thus, TPEN may be used to reversibly manipulate store calcium levels without interfering with other aspects of calcium homeostasis. lonophores, e.g., ionomycin (a Ca²⁺/proton ionophore), also may be used for active depletion of intracellular calcium stores by embedding in the ER membrane and providing a pore-like mechanism for movement of calcium out of the ER.

(2) Analysis of Ion Flux

Determining whether a cell exhibits store-operated calcium entry, or determining calcium movement into, out of or within a cell in general, can include a step of detecting and/or analyzing ion flux into cells and/or the cytoplasm or across a membrane. Techniques for detection and analysis of ion flux into cells are well known in the art. Typically, when using ion flux as a way to determine whether a cell exhibits store-operated calcium entry, cells may be exposed to conditions that activate store-operated calcium entry, including those described herein, such as treatment with an agonist that results in generation of IP₃, internal perfusion with IP₃, treatment with a store-depletion agent e.g., thapsigargin, or buffering of cytoplasmic calcium, and are then analyzed for evidence of movement of ions out of intracellular organelles and/or movement of ions into the cells. Depending on the assay conditions, the ion may be a calcium or other cation, for example sodium or manganese, that can be transported via a store-operated ion transport protein.

For example, ion flux, such as that which occurs in store-operated calcium entry, can be measured electrophysiologically using patch clamp methods [see, e.g., Hamil et al.(1981) Pflugers Arch, 391:85-100; Hoffman et al. (1999) Nature 397:259-263; and Krause et al. (1999) J. Biol. Chem. 274:36957-36962] to record the inward Ca²⁺ current. The current may be highly selective for Ca²⁺ divalent ions, may display negative feedback regulation by Ca²⁺ [see, e.g., Zweifach and Lewis (1995) J. Gen. Physiol. 105:209-226], and may be inhibited by divalent and trivalent metal ions such as Zn²⁺, Ni²⁺, Gd³⁺ and La³⁺[Parekh and Penner (1997) Physiol. Rev. 77:901-930]. Negative feedback by Ca²⁺ can be eliminated by the inclusion of very high concentrations of Ca²⁺ buffers in the patch pipet. In the absence of these buffers or with buffers of lower capacity, the current may be too small to be detected, although Ca²⁺ entry clearly occurs. The current may be strongly inwardly rectifying and may lose its property of inward rectification in the complete absence of divalent cations. It may be possible to determine the time course of activation of the current in single cells followed by passive store depletion via patch pipettes containing Ca²⁺ chelating reagent BAPTA in the whole cell configuration using Na²⁺ used as the charge carrier [Kerschbaum and Cahalan (1999) Science 283:836].

The electrophysiological measurement/recording of currents associated with store-operated ion flux through a membrane, e.g., currents through store-operated ion channels, can provide for the assessment of such biophysiological properties as kinetics, voltage dependence, ionic selectivity. The electrophysiological measurement can be performed by using whole cell patch clamp methods which can allow for the reliable and prescise determination of the conditions under which Ca²⁺ influx occurs. As described by Hofman et al. [(1999) Nature 397:259-263], the patch clamp technique can be used in whole-cell, cell-attached and inside-out mode.

Additionally patch clamp methods can be performed in a tight seal whole cell, configuration. Variations of the patch-clamp technique or other methods for detecting and analyzing ionic activity of cells, which are routine in the art, can also be used.

Measurement of changes of intracellular ions, such as cations including Ca²⁺, can also can also be performed using fluorescence imaging, such as fluorescence videomicroscopy, digital imaging or ratioimaging techniques. Measurement of changes in intracellular Ca²⁺ ([Ca²⁺]ᵢ) in individual cells by fluorescence videomicroscopy can be performed using digital imaging system such as that produced by T.I.L.L.

Photonics or the Attoflour Digital Imaging and Photometry attachment of a Carl Zeis axiovert inverted microscope. Typically cells can be grown on coverslips, rinsed and incubated with 5 μM fura2/AM (Molecular Probes) at 37° C. for 30 minutes and then washed with HPSS. The coverslips with the cells are then typically clamped into a circular open-bottom chamber and mounted onto the stage of a microscope. [Ca²⁺]ᵢ can be calculated from fluorescence ratios obtained at 340 and 380 nm excitation wavelengths [Garcia et. al (1994) J. Neurosci. 14: 1233-1246]. Modified protocols such as those described by Zitt et. al. [(1997) J. Cell Biol. 138: 1333-1341] and alternate forms of fluorescence ratioimaging may also be used.

Calcium-sensitive indicators, such as fluo-3 (e.g., Catalog No. F-1241, Molecular Probes, Inc., Eugene, Oreg.), are available as acetoxymethyl esters which are membrane permeable. When the acetoxymethyl ester form of the indicator enters a cell, the ester group is removed by cytosolic esterases, thereby trapping the free indicator in the cytosol. Interaction of the free indicator with calcium results in increased fluorescence of the indicator; therefore, an increase in the intracellular Ca²⁺ concentration of cells containing the indicator can be expressed directly as an increase in fluorescence.

Additionally, calcium flux in a cell may be monitored using a reporter expression system. In such a system, the cell in which calcium levels and fluctuations therein are monitored may contain a reporter gene encoding a detectable signal, such as luciferase, which is linked to a transcription regulatory element, e.g., promoter, that is induced by calcium-dependent factors.

3. Reduction, Alteration or Elimination of the Expression of One or More Genes in a Cell

In the methods described herein for identifying a protein (and/or nucleic acid encoding a protein) involved in modulating intracellular calcium, a cell is subjected to conditions whereby the expression of one or more genes in the cell is reduced, altered or eliminated. The resulting cell is analyzed to determine the effects of reduction, alteration or elimination of gene expression on intracellular calcium. A protein (and/or nucleic acid encoding a protein) that can modulate intracellular calcium is identified by a cell in which reduction, alteration or elimination of the expression of a gene is accompanied by an alteration in intracellular calcium and/or calcium (or other cation or charge carrier) movement into, out of or within a cell.

The methods provided herein for identifying a protein (and/or nucleic acid encoding a protein) involved in modulating intracellular calcium are function-based and, importantly, specific; that is, they provide a direct, one-to-one correlation between a protein (and/or nucleic acid encoding a protein) that can be one expressed in its native cellular environment and cell calcium, and, in particular embodiments, resting cytosolic calcium levels, intracellular organelle calcium levels/uptake/release and/or store-operated ion flux. This correlation is determined by the association of altered expression of a gene with alterations in cell calcium and/or movement of calcium (or other cation or charge carrier) into, out of or within cells, such as, for example, store-operated ion flux or intracellular organelle (e.g., endoplasmic reticulum) calcium levels, uptake or release. Thus, for example, particular embodiments of the methods provided herein involve identification of a gene that, when expressed in an altered fashion (including reduced or eliminated expression), results in altered, reduced or eliminated store-operated ion entry in a cell that contains the gene. The protein encoded by the identified gene is thereby identified as an intracellular calcium-modulating protein, such as, for example, an ion transport protein, involved in maintenance of resting cytosolic calcium levels, intracellular organelle calcium storage, uptake or release and/or store-operated calcium entry.

Techniques for altering gene expression in a cell are known in the art and described herein. Any such procedures may be used in intracellular calcium-modulating protein identification methods provided herein. With respect to these methods, the alteration in gene expression need only be such that any associated alteration in cell calcium and/or calcium (or other cation or charge carrier) into, out of or within a cell, e.g., store-operated calcium entry, is detectable.

The alteration may be one that completely or nearly completely eliminates expression of a gene in the cell. If the gene is one that encodes a protein involved in maintenance of resting cytosolic calcium levels in the cell, then it may be identified, for example, by an alteration of resting cytosolic calcium levels in a cell in which expression of the gene has been eliminated. An alteration of resting cytosolic calcium levels includes increases and decreases in resting cytosolic calcium levels. If the gene is one that encodes a protein that is principally involved in providing for store-operated calcium influx in the cell, then it may be identified through complete or nearly complete elimination of store-operated ion flux into the cell. Furthermore, if the gene is one that encodes a protein that substantially participates in but is not principally involved in providing for store-operated calcium influx into the cell, then completely eliminating expression of the gene may identify the gene by an alteration and/or reduction, but perhaps not complete elimination, of store-operated ion flux into the cell. For example, if a gene encodes a protein that is involved in modulating or regulating store-operated calcium entry or encodes an ion transport protein that provides for or is involved in store-operated calcium influx by being a component, such as a subunit, of a multi-subunit complex (e.g., heteromeric complex) providing for store-operated calcium influx, then complete or near complete elimination of expression of the gene may result in altered store-operated ion current properties, e.g., ion selectivity and/or conductance, reduced or even increased store-operated ion flux into the cell. If the gene is one that encodes a protein that is involved in modulating intracellular organelle (e.g., endoplasmic reticulum) calcium, then complete or near complete elimination of expression of the gene may result in an alteration in the calcium level of the organelle and/or in complete or nearly complete elimination of calcium movement into or out of the organelle (e.g., if the gene encodes an organelle ion transport protein) or in altered properties of the movement of calcium into or out of the organelle (e.g., if the gene encodes a protein that regulates or modulates, or that is not principally involved in providing for, calcium movement into or out of the organelle).

The alteration in gene expression may be one that only partially eliminates expression of a gene in the cell. In such an instance, for example, if the gene is one that encodes a protein that is principally involved in providing for store-operated calcium influx in the cell, then it may be identified through a partial reduction of store-operated ion flux into the cell. If the gene is one that encodes a protein that substantially participates in but is not principally involved in providing for store-operated calcium influx into the cell, then partial elimination of expression of the gene may identify the gene by an alteration and/or reduction of store-operated ion flux into the cell.

The methods include embodiments where the expression of two or more genes, or a “pool” of genes is altered in a cell. Thus, for example, if a combination of the genes encodes a combination of components, each of which participates in an ion transport process that provides for store-operated calcium influx into a cell, then complete or nearly complete elimination of expression of the combination of genes may identify the combination of genes by complete or nearly complete elimination of store-operated ion flux into the cell.

Thus, an “alteration” of cell calcium or calcium movement into, out of or within a cell can be a complete or nearly complete elimination of the activity, an alteration in properties or characteristics of the activity or an increase in the activity. Similarly, an “alteration” in gene expression may be complete or nearly complete elimination of the expression of a gene, a reduction in the expression of a gene, an increase in the expression of a gene, or an alteration in the protein encoded by the gene (such as truncation or other alteration that effectively renders the protein nonfunctional or provides for aberrant functioning of the protein).

Methods for assessing the type and extent of an alteration in gene expression are known in the art. For example, the level and characteristics (e.g., size) of a transcript (e.g., mRNA) generated from a gene can be evaluated by Northern blot analysis employing nucleic acid probes that specifically hybridize to the transcript or by reverse transcriptase PCR (RT-PCR) nucleic acid amplification. The level and characteristics (e.g., size and immunoreactivity) of a protein encoded by a gene can be evaluated by western blot or other immunoassays employing antibodies that specifically bind the protein.

a. RNA-Based Methods of Altering Gene Expression

(1) Antisense Methods

Introduction into a cell of nucleic acid complementary to an RNA transcript encoded by a gene within the cell can be used to alter expression of the gene. Such an approach may be referred to as an “antisense” RNA method when a single-stranded nucleic acid molecule is introduced into a cell [see, e.g., Izant and Weintraub (1984) Cell 36:1007-1015]. RNA can be synthesized from, for example, phagemid clones containing DNA corresponding to a gene to be targeted for alteration of expression, using T3 and T7 polymerase. DNA templates may be removed by DNase treatments. Antisense RNA is then introduced into a cell and after an appropriate period, store-operated ion flux of the cell is evaluated and compared to such ion flux in the cell prior to introduction of antisense RNA or to store-operated ion flux in a substantially similar cell that has not received antisense RNA. Antisense RNA may also be expressed in a cell by transfecting the cell with a plasmid containing nucleic acid coding for antisense RNA.

(2) RNA Interference (RNAi) Methods

RNA interference (RNAi) is a method of gene silencing which involves the introduction of double-stranded RNA (dsRNA) into cells. The basic premise of RNAi is the ability of double-stranded RNA (dsRNA) to specifically block expression of its homologous gene when present in cells. Thus, in performing RNAi, a dsRNA construct containing a nucleotide sequence with homology to or identical to a portion of the target gene to be silenced is introduced into or generated within a cell containing the target gene. Generally, in the RNAi reaction, both strands (sense and antisense) of the dsRNA are processed to small RNA fragments or segments (referred to as small interfering RNAs, siRNAs or guide RNAs) of from about 21-23 nucleotides (nt) in length. Processing of the dsRNA to the small RNA fragments does not require the targeted mRNA, which demonstrates that the small RNA species is generated by processing of the dsRNA and not as a product of dsRNA-targeted mRNA degradation. The mRNA is cleaved only within the region of identity with the dsRNA. Cleavage occurs at sites 21-23 nucleotides apart, the same interval observed for the dsRNA itself, suggesting that the 21-23 nucleotide fragments from the dsRNA are guiding mRNA cleavage. The RNAi phenomenon is mediated by a set of enzyme activities that are evolutionarily conserved in eukaryotes ranging from plants to mammals.

After partial purification, a multi-component nuclease (RISC nuclease) co-fractionates with the dsRNA fragments which may confer specificity to the nuclease through homology to the substrate mRNAs. It is believed that the dsRNA fragments instruct the RISC nuclease to destroy specific mRNAs corresponding to the dsRNA sequences. An additional enzyme, Dicer, has been identified that can produce the guide RNAs. Dicer is a member of the RNAse III family of nucleases that specifically cleave dsRNA and is evolutionarily conserved in worms, flies, plants, fungi and mammals. The enzyme has a distinctive structure which includes a helicase domain and dual RNAse III motifs. Dicer also contains a region of homology to the RDE1/QDE2/ARGONAUTE family, which have been genetically linked to RNAi in lower eukaryotes. Activation, or overexpression, of Dicer and/or Argonaute is, thus, useful for facilitating RNAi in cells, such as cultured eukaryotic cells, or mammalian cells in culture or in whole organisms.

Mammalian cells exhibit an interferon-mediated antiviral response to long dsRNA that results in diminished protein synthesis. This response makes it difficult to utilize long dsRNA in RNA interference of mammalian cells. Thus, for RNAi of mammalian cells, short interfering dsRNAs of about 21 nucleotides can be used which do not activate the antiviral response [see, e.g., Elbashir et al. (2001) Nature 411:494-498]. Additionally, cells can be treated with an agent(s) that inhibits the double-stranded RNA-dependent protein known as PKR (protein kinase RNA-activated). Part of the interferon response is the activation of the PKR response. PKR phosphorylates and inactivates elF2a. Inactivation of elF2α results in inhibition of protein synthesis and ultimately apoptosis. This sequence-independent PKR response can be overcome in favor of the sequence-specific RNAi response without altering the activity of PKR; however, in certain instances, it may be desirable to treat the cells with agents which inhibit expression of PKR, cause its destruction, and/or inhibit the kinase activity of PKR. Likewise, overexpression of an agent which ectopically activates elF2a can be used.

The double-stranded structure may be formed by a single self-complementary RNA strand or two complementary RNA strands. The dsRNA construct may include modifications to either the phosphate-sugar backbone or the nucleoside. The backbone may be modified for stability or for other reasons. The phosphodiester linkages may be modified to include at least one of a nitrogen or sulfur heteroatom. RNA duplex formation may be initiated either inside or outside the cell. The RNA may be introduced in an amount which allows delivery of at least one copy per cell. Higher doses (e.g., at least 5, 10, 100, 500, or 1000 copies per cell) of double-stranded material may yield more effective inhibition; lower doses may be useful for specific applications. Inhibition is sequence-specific in that nucleotide sequences corresponding to the duplex region of the RNA are targeted for genetic inhibition.

Double-stranded RNA constructs containing a nucleotide sequence identical to a portion of the target gene are generally most effective in the inhibition of target gene expression. RNA sequences with insertions, deletions, and single point mutations relative to the target sequence have also been found to be effective for inhibition. Sequence identity may be optimized by sequence comparison and alignment algorithms known in the art [see e.g., Gribskov and Devereux, Sequence Analysis Primer, Stickton Press, 1991] and calculating the percent difference between the nucleotide sequences by, for example, the Smith-Waterman algorithm as inplemented in the BESTFIT software program using default parameters (e.g., University of Wisconsin Genetic Computing Group). Greater than 90% sequence identity, or even 100% sequence identity, generally provides for the greatest inhibition; however, it is not required for inhibition. The RNAi method is able to tolerate sequence variations that might be expected due to genetic mutation, strain polymorphism, or evolutionary divergence. Alternatively, the duplex region of the RNA may be defined functionally as a nucleotide sequence that is capable of hybridizing with a portion of the target gene transcript. The length of the identical nucleotide sequences may be, for example, at least 25, 50, 100, 200, 300, or 400 bases.

The dsRNA construct may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the dsRNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. The dsRNA construct may be chemically or enzymatically synthesized by manual or automated reactions. The dsRNA construct may be synthesized by a cellular RNA polymerase or a bacteriophage RNA polymerase (e.g., T3, T7, SP6). The use and production of an expression construct are known in the art [see, e.g., WO97/32016; U.S. Pat. Nos. 5,593,874, 5,698,425, 5,712,135, 5,789,214, and 5,804,693]. If synthesized chemically or by in vitro enzymatic synthesis, the RNA may be purified prior to introduction into the cell. For example, RNA can be purified from a mixture by extraction with a solvent or resin, precipitation, electrophoresis, chromatography or a combination thereof. Alternatively, the dsRNA construct may be used with no or a minimum of purification to avoid losses due to sample processing. The dsRNA construct may be dried for storage or dissolved in an aqueous solution. The solution may contain buffers or salts to promote annealing, and/or stabilization of the duplex strands.

RNAi can be used to alter gene expression in a cell derived from or contained in any organism. The organism may be a plant, animal, protozoan, bacterium, virus, or fungus. For example, such organisms include, but are not limited to Drosophila, trypanasomes, lanaria, hydra, zebrafish, Caenorhabditis elegans, mice, human and other mammals. The cell may be from, for example, the germ line or somatic, totipotent or pluripotent, dividing or non-dividing, parenchyma or epithelium, immortalized or transformed, a stem cell or a differentiated cell. The cell may be any individual cell of the early embryo, and may be a blastocyte, or, alternatively, it may be an oocyte [see, e.g., Fire et al. (1998) Nature 391:806; Clemens et al. (2000) Proc. Natl. Acad. Sci. U.S.A. 97:6499-6503; PCT International Application Publication Nos. WO01/36646, WO99/32619, WO01/68836, WO01/29058 and WO01/75164].

The dsRNA may be directly injected into the cell or may be introduced by bathing the cell in a solution containing RNA. Other methods for introducing dsRNA intracellularly include bombardment by particles covered by the RNA, for example gene gun technology in which the dsRNA is immobilized on gold particles and fired directly at the site, and electroporation of cell membranes in the presence of the RNA. Precise conditions for electroporation depend on the device used to produce the electro-shock and the dimensions of the chamber used to hold the cells. This method permits RNAi on a large scale. Any known gene therapy technique can also be used to administer the RNA. A viral construct packaged into a viral particle would accomplish both efficient introduction of an expression construct into the cell and transcription of RNA encoded by the expression construct. Other methods known in the art for introducing nucleic acids into cells may be used, such as lipid-mediated carrier transport, chemical-mediated transport, such as calcium phosphate, and the like. Thus, the RNA may be introduced along with components that perform one or more of the following activities: enhance RNA uptake by the cell, promote annealing of the duplex strands, stabilize the annealed strands, or otherwise increase inhibition of the target gene. A transgenic animal that expresses RNA from a recombinant construct may be produced by introducing the construct into a zygote, an embryonic stem cell, or another multipotent cell derived from the appropriate animal.

RNAi may also be perfomed on an organismal level. Mammalian cells can respond to extracellular dsRNA, and RNAi can act systemically; therefore a specific transport mechanism for dsRNA may exist [see, e.g., Asher et al. (1969) Nature 223:715-717; WO01/36646; WO01/68836]. Consequently, injection of dsRNA into one tissue can inhibit gene function in cells throughout the animal. Thus, dsRNA may be administered extracellularly into a cavity, interstitial space, into the circulation of a mammal, introduced orally, or may be introduced by bathing an organism in a solution containing RNA. Methods for oral introduction include direct mixing of the RNA with food of the organism, as well as engineered approaches in which a species that is used as food is engineered to express the RNA, then fed to the organism to be affected. For example, food bacteria, such as Lactococcus lactis, may be transformed to produce the dsRNA [see, e.g., WO97/17117, WO97/14806]. Methods of injection include injection into vascular or extravascular circulation, the blood or lymph systems and the cerebrospinal fluid are sites where the RNA may be injected.

Drosophila cells are particularly well-suited for RNAi-based alteration of gene expression. Many Drosophila cell lines have been established and can be biochemically characterized for use in studying various cellular processes. Drosophila cell lines that are known to respond to dsRNAs by ablating expression of the target protein include S2, KC, BG2-C6, and Shi cells. Many signal transduction pathways and other cellular processes have been highly conserved from Drosophila to mammals, making it possible to study complex biochemical problems in a genetically tractable model organism. Importantly, results obtained from the cell culture studies can be confirmed in the whole organism, because Drosophila is very amenable to RNAi analyses at the organismal level. The use of dsRNA in Drosophila cell culture to silence expression of specific genes is technically simple, efficacious, and highly reproducible. The dsRNAs are efficiently internalized by the cells, thereby circumventing the problems generated by variable transfection efficiencies. Also, the gene silencing effect can be sustained through many cell divisions.

Compared to antisense technology, RNAi has been reported to achieve greater than 95% reduction in gene product. This effect can be manifested over a period of 6-7 days, thus allowing for many data points and repetition of the assay over time (Caplen et al. Gene 252:95-105 (2000)).

b. Gene Knock Out or Deletion

Direct gene “knock-out” procedures may also be used to alter the expression of one or more genes in a cell. In these methods, homologous recombination between DNA in a cell and heterologous nucleic acid introduced into the cell results in elimination of a targeted gene from the genome or alteration of the gene such that it does not produce functional protein. Methods of designing nucleic acid constructs for use in targeted gene disruption or deletion are well known in the art [see, e.g., Capecchi (1989) Science 244:1288; Capecchi et al. Nature (1990) 344:105; Koller et al. (1990) Science 248:1227].

C. Gene Insertion

Transfection methods may be used to introduce one or more genes into a host cell. The nucleic acid(s) transferred into the host cell may encode a wild-type or altered protein or a domain, derivative, fragment or homolog thereof. Transfer of nucleic acid(s) into a host cell can be accomplished by a variety of procedures. Such procedures include, but are not limited to, direct uptake using calcium phosphate [CaPO₄; see, e.g., Wigler et al. (1979) Proc. Natl. Acad. Sci. U.S.A. 76:1373-1376], polyethylene glycol [PEG]-mediated DNA uptake, electroporation, lipofection [see, e.g., Strauss (1996) Meth. Mol. Biol. 54:307-327], microcell fusion [see, e.g., Lambert (1991) Proc. Natl. Acad. Sci. U.S.A. 88:5907-5911; U.S. Pat. No. 5,396,767, Sawford et al. (1987) Somatic Cell Mol. Genet. 13:279-284; Dhar et al. (1984) Somatic Cell Mol. Genet. 10:547-559; and McNeill-Killary et al. (1995) Meth. Enzymol. 254:133-152], lipid-mediated carrier systems [see, e.g., Teifel et al. (1995) Biotechniques 19:79-80; Albrecht et al. (1996) Ann. Hematol. 72:73-79; Holmen et al. (1995) In Vitro Cell Dev. Biol. Anim. 31:347-351; Remy et al. (1994) Bioconjug. Chem. 5:647-654; Le Bolch et al. (1995) Tetrahedron Lett. 3:6681-6684; Loeffler et al. (1993) Meth. Enzymol. 217:599-618], liposome-mediated delivery [see, e.g., Philip et al. (1993) J. Biol. Chem. 268:16087-16090 and Aksentijevich et al. (1996) Hum. Gen. Ther. 7:1111-1122], adenovirus infection [see, e.g., Ragot et al. (1993) Nature 361:647-650], retroviral transduction [see, e.g., Cochlovius et al. (1998) Cancer Immunol. Immunother. 46:61-66, Bunnell et al. (1995) Proc. Natl. Acad. Sci. U.S.A. 92:7739 and Finer et al. (1994) Blood 83:43], electroporation [see, e.g., Hughes et al. (1996) J. Biol. Chem. 271:5369-5377 and Cron et al. (1997) J. Immunol. Meth. 205:145-1501, particle bombardment [see, e.g., Yang et al. (1990) Proc. Natl. Acad. Sci. U.S.A. 87:9568-9572], direct local injection of the DNA (for in vivo transfer of DNA) [see, e.g., Wolff et al. (1990) Science 247:1465-1468; and Zhu et al. (1993) Science 261:209-211], antibody-based methods [Mannino and Gould-Fogerite (1988) Biotechniques 6:682-690], retroviruses [Roux et al. (1989) Proc. Natl. Acad. Sci. U.S.A. 86:9079-9083], and antifection [see, e.g., Hirsch et al. (1993) Transpl. Proc. 25:138-139 and Poncet et al. (1996) Gene Therapy 3:731-738].

The nucleic acid encoding a protein of interest can be operably linked to elements that facilitate expression of the nucleic acid in host cells. Such elements include promoters, enhancers and terminators that are functional in the recipient host cell and are known to those of skill in the art.

For transfer of nucleic acid encoding a protein of interest into cells, the nucleic acid may be contained within a vector. Any vector known in the art for transfer and expression of nucleic acids in cells may be used, including plasmids, cosmids and artificial chromosomes. For simultaneous co-transfection, more than one protein of interest may be contained on separate vectors or on the same vector in which they can be operably linked to elements that facilitate expression of the nucleic acids in host cells. Multiple sequences, such as an nucleic acids expressing multiple elements in a calcium flux pathway, contained on the same vector may be controlled either by a single promoter or by multiple promoters. In a specific embodiment, the promoter is not native to the gene(s) expressing the protien(s).

Any methods known to those of skill in the art for the insertion of DNA fragments into a vector may be used to construct expression vectors containing a protein coding sequence of interest and appropriate transcriptional/translational control signals and/or other protein coding sequences. These methods may include in vitro recombinant DNA and synthetic techniques and in vivo recombinants (genetic recombination).

4. Determining Effects of Alteration of Gene Expression on Intracellular Calcium

Following or simultaneously with the alteration of gene expression in a cell, the cell is analyzed to evaluate the effect, if any, on intracellular calcium. Intracellular calcium may be evaluated in a number of ways, including any of the methods described herein or known in the art. For example, intracellular calcium can be evaluated by assessment of cytosolic or intracellular organelle (e.g., endoplasmic reticulum) calcium levels and/or fluxes or by assessment of movement into, out of or within the cell following reduction, alteration or elimination of the expression of one or more genes. Cells can be exposed to conditions (e.g., intracellular and/or extracellular calcium buffering, including use of calcium chelators, and exposure to agents that activate, inhibit or otherwise modulate various cation entry/flux processes) that facilitate assessment of intracellular calcium. In particular embodiments of the methods provided herein for identifying intracellular calcium-modulating proteins, the resting cytosolic calcium levels, store-operated calcium entry into the cell and/or intracellular organelle calcium levels, uptake or release are evaluated during and/or following alteration of gene expression in a cell. For example, calcium levels and/or calcium release from the endoplasmic reticulum can directly be assessed using mag-fura 2, endoplasmic reticulum-targeted aequorin or cameleons. One method for indirect assessment of calcium levels or release is monitoring intracellular calcium levels (for example using fluorescence-based methods) after exposing a cell to an agent that effects calcium release (actively, e.g., IP₃, or passively, e.g., thapsigargin) from the organelle in the absence of extracellular calcium.

Resting cytosolic calcium levels, intracellular organelle calcium levels and cation movement may be assessed using any of the methods described herein or known in the art (see, e.g., descriptions herein of calcium-sensitive indicator-based measurements, such as fluo-3, mag-fura 2 and ER-targeted aequorin, labelled calcium (such as ⁴⁵Ca²⁺)-based measurements, and electrophysiological measurements). Particular aspects of ion flux that may be assessed include, but are not limited to, a reduction (including elimination) or increase in the amount of ion flux, altered biophysical properties of the ion current, and altered sensitivities of the flux to activators or inhibitors of calcium flux processes, such as, for example, store-operated calcium entry.

A protein, such as an ion transport protein, that is involved in, participates in and/or provides for store-operated calcium entry, and/or nucleic acid encoding such a protein, can be identified by a cell in which reduction, alteration or elimination of the expression of a gene is accompanied by an alteration, e.g., reduction, elimination, increase or other modification of store-operated ion flux into the cell. A protein, for example, an ion transport protein, involved in maintenance of resting cytosolic calcium levels, and/or nucleic acid encoding such a protein, can be identified by a cell in which reduction, alteration or elimination of the expression of a gene is accompanied by an alteration, e.g., increase, decrease or other modification, of resting cytosolic calcium levels. A protein, for example an ion transport protein, involved in intracellular organelle calcium storage, uptake or release (and/or nucleic acid encoding such a protein) can be identified by a cell in which reduction, alteration or elimination of the expression of a gene is accompanied by an alteration, e.g., increase, decrease, reduction, elimination or other modification, of the calcium levels of an intracellular organelle or of movement of cations (or other charge carrier) into or out of an intracellular organelle.

5. Methods of Screening for Candidate Intracellular Calcium-Modulating Proteins

In conducting the method for identifying an ion transport or other intracellular calcium-modulating protein and/or nucleic acid encoding such a protein, the gene or genes that is/are altered in expression may be preselected, specific genes or may be random. For example, in certain embodiments of the methods, such as methods utilizing antisense RNA or RNA interference, the RNA used in effecting the alteration in gene expression could be generated from a random collection of DNA sequences obtained from a cell of the type in which the alteration is being conducted.

Alternatively, particular genes may be targeted for alteration of expression. Because most cells contain a vast number of genes, it can be desirable to identify candidate genes encoding proteins that may modulate intracellular calcium, for example, proteins involved in maintenance of cytosolic calcium levels, proteins that are involved in, participate in and/or provide for store-operated calcium entry or proteins involved in intracellular organelle calcium storage, uptake or release for targeted alteration of gene expression. Provided herein are methods for screening for candidate proteins (and/or genes or nucleic acids encoding proteins) involved in intracellular calcium modulation. In a particular embodiment, the method involves screening for candidate store-operated calcium entry proteins and/or genes or nucleic acids encoding such proteins. The methods can be used in conjunction with the methods of identifying a protein (and/or nucleic acid encoding a protein) involved in modulating intracellular calcium. Thus, in particular embodiments of the methods for identifying intracellular calcium-modulating proteins provided herein, the method includes alteration of the expression of one or more genes separately and/or in combinations that have been selected as genes encoding candidate ion transport proteins, calcium-binding proteins, EF hand-containing proteins or P-loop containing proteins that may be involved in intracellular calcium modulation. In a particular method, the cell utilized is a Drosophila cell, such as, for example, an S2 cell.

One embodiment of the methods of screening for candidate intracellular calcium-modulating proteins includes a step of evaluating a protein (and/or an amino acid sequence encoded by a nucleic acid or gene) for the presence of one or more, or two or more, or three or more, or four or more, or five or more, or six or more domains, each of which has homology to an amino acid sequence characteristic of an amino acid sequence that extends through a cellular membrane (e.g., a transmembrane domain). Characteristic features of transmembrane domains, e.g., hydrophobicity and helical conformation, are described herein and known in the art. The extent of homology suitable for use in the screening method can depend on several factors (e.g., the number of candidates desired and whether other structural motifs, such as described herein, are to be considered for inclusion in selecting candidate proteins), and can be empirically determined. The proteins or encoded amino acid sequences may be screened only for the presence of sequences with homology to transmembrane domains or may be screened for the presence of such domains and for the presence of sequences with homology to one or more other motifs or domains. One method for identifying proteins with homology to transmembrane domains is by evaluating amino acid sequence of a protein for the presence of predicted transmembrane domains using the TMpred program (see, e.g., www.ch.embnet.org/software/TMpred_form.html which can be accessed via the ExPASy Molecular Biology Server (www.expasy.ch)). The TMpred program predicts membrane-spanning regions and their orientation through use of an algorithm generated by statistical analysis of naturally occurring transmembrane proteins (i.e., proteins contained in the TMbase database; see Hofman and Stoffel (1993) Biol. Chem. Hoppe-Seyler 374:166).

In a further embodiment of the methods for screening for candidate intracellular calcium-modulating proteins, a protein (and/or amino acid sequence encoded by a nucleic acid or gene) is evaluated for the presence of amino acid sequence(s) with homology to amino acid sequence characteristic of a protein that participates in the transport of calcium, such as a calcium channel. Characteristic features of such sequences, e.g., amphipathic segments, are described herein and known in the art. The proteins may be screened only for the presence of such domains or may be screened for the presence of such domains and for the presence of one, or more, or multiple, e.g., 6 or more, domains with homology to a transmembrane domain. In one method, the amino acid sequence characteristic of a protein that transports calcium can include an amino acid sequence characteristic of any one or more one of the following proteins and/or domains: ion channels, cation channels, and calcium or sodium pores. In a particular method, the screening comprises evaluating proteins for homology with amino acid sequences characteristic of a protein that participates in calcium transport and for the absence of one or both of the following amino acid sequences: EWKFAR and EXD(E)CR(K)GXYXXYE (wherein “X” represents any amino acid, and an amino acid residue in parentheses is an alternative to the residue immediately preceeding it). The amino acid sequence EWKFAR is a motif characteristic of transient receptor potential (trp) calcium channel proteins. The sequence of amino acids EXD(E)CR(K)GXYXXYE is a motif characteristic of a pore-forming region of calcium channels such as voltage-gated calcium channels. This sequence of amino acids [EXD(E)CR(K)GXYXXYE] is a motif characteristic of a pore-forming region of calcium channels such as voltage-gated calcium channels.

In another embodiment of the methods for screening for candidate intracellular calcium-modulating proteins, a protein (and/or an amino acid sequence encoded by a nucleic acid or gene) is evaluated for the presence of amino acid sequence with homology to an amino acid sequence characteristic of a calcium-binding motif. One such calcium binding motif is referred to as an EF-hand [see, e.g., Kretsinger (1997) Nat. Struct. Biol. 4:514-516; Ikura (1996) Trends Biochem. Sci. 21:14-17; Kawasaki et al. (1995) Protein Profile 2:297-490]. Characteristic features of EF-hand motifs include those described herein and known in the art. Typically, an EF-hand motif contains a loop of about 12 amino acid residues flanked on either side by an alpha helix of about 12 amino acid residues. Position 12 of the loop typically contains a Glu or Asp. EF-hands may undergo a conformational change upon binding calcium ions. Proteins may be screened only for homology with calcium-binding domains or may be screened for the homology to such domains and for the presence of one, or more, or multiple, e.g., 6 or more, domains with homology to transmembrane domains and/or for homology with an amino acid sequence characteristic of a protein that transports calcium, such as a calcium channel.

In another embodiment of the methods for screening for candidate intracellular calcium-modulating proteins, a protein (and/or an amino acid sequence encoded by a nucleic acid or gene) is evaluated for the presence of amino acid sequence(s) having homology to an amino acid sequence characteristic of a presenilin protein. Presenilins are polytopic transmembrane proteins containing about six to nine transmembrane domains and a large hydrophilic loop towards the C-terminal part of the protein. The encoded proteins may be screened only for homology to presenilin domains, e.g., the loop domain, or may be screened for homology to such domains and for homology to calcium-binding domains, one, or more, or multiple, e.g., 6 or more, transmembrane domains and/or an amino acid sequence characteristic of a protein that transports calcium, such as a calcium channel.

In particular embodiments of any of the methods for screening for candidate intracellular calcium-modulating proteins provided herein, the protein (or amino acid sequence encoded by a gene or nucleic acid) screened is from an insect, e.g., Drosophila or Anopheles, nematode, e.g., Caenorhabditis, yeast, fish, e.g., zebrafish and pufferfish, mammal, mouse, rat or human. In particular embodiments, the protein (or amino acid sequence encoded by a gene or nucleic acid) is from Drosophila

Proteins (and/or amino acid sequences encoded by genes or nucleic acids) that can be evaluated in the screening methods include those contained in protein and gene/nucleic acid databases, such as those generated in connection with genome-sequencing projects. Proteins and/or amino acid sequences encoded by genes/nucleic acids contained in the databases can be evaluated in accordance with the criteria set forth above in order to identify candidate proteins involved in intracellular calcium modulation. The evaluation can be performed, for example, through computer-assisted database searches. Such databases and search systems are known in the art. For example, a database of the Drosophila genome and system for searching genes and encoded proteins of the genome is referred to as the “FlyBase GadFly Genome.Annotation Database” Drosophila [see, e.g., www.fruitfly.org or hedgehog.lbl.gov]. Other databases include C. elegans, yeast, human, mouse, rat and others. Amino acid sequences characteristic of the selected criteria (including, for example, sequences characteristic of ion channels, cation channels, and calcium or sodium pores) are incorporated into the annotation and search tools of many protein and genomic databases. Computer-assisted proteome analysis programs have been established from many different genomes (see, e.g., www.ebi.ak.uk/proteome). Additionally, amino acid sequence pattern information is available and searchable through other databases, including InterPro [see www.ebi.ac.uk/interpro/] as well as SWISS-PROT, TrEMBL, PROSITE, PRINTS, Pfam and Prodom.

Provided herein is a method for identifying a protein involved in modulating intracellular calcium, comprising:

reducing, altering or eliminating expression of one or more genes in a cell; and

evaluating intracellular calcium in the cell, whereby one or more proteins involved in modulating intracellular calcium and/or one or more nucleic acids encoding one or more proteins involved in modulating intracellular calcium is identified in cells in which a reduction, alteration or elimination of gene expression is associated with an alteration in intracellular calcium in the cell. In this method, intracellular calcium may be evaluated in any of a number of ways, including, but not limited to, measurement of intracellular calcium levels and/or monitoring intracellular calcium level time courses.

Also provided herein is a method for identifying a protein involved in modulating intracellular calcium, comprising:

reducing, altering or eliminating expression of one or more genes in a cell; and

evaluating store-operated calcium entry into the cell, whereby one or more proteins involved in modulating intracellular calcium and/or one or more nucleic acids encoding one or more proteins involved in modulating intracellular calcium is identified in cells in which a reduction, alteration or elimination of gene expression is associated with a reduction, alteration or elimination of store-operated calcium entry in the cell; and wherein

prior to reduction, alteration or elimination of gene expression, the cell exhibits store-operated calcium entry.

Further provided herein is a method for identifying a protein involved in modulating intracellular calcium, comprising:

reducing, altering or eliminating expression of one or more genes in a cell; and

evaluating cytosolic calcium levels, whereby one or more proteins involved in modulating intracellular calcium and/or one or more nucleic acids encoding one or more proteins involved in modulating intracellular calcium is identified in cells in which a reduction, alteration or elimination of gene expression is associated with an alteration in cytosolic calcium levels.

Another method for identifying a protein involved in modulating intracellular calcium comprises:

reducing, altering or eliminating expression of one or more genes in a cell; and

determining intracellular calcium levels in the resulting cell after reduction of calcium levels in an intracellular calcium store, whereby one or more proteins involved in modulating intracellular calcium and/or one or more nucleic acids encoding one or more proteins involved in modulating intracellular calcium is identified in cells in which a reduction, alteration or elimination of gene expression is associated with a reduction, alteration or elimination of an increase in cytosolic calcium levels after reduction of calcium levels of intracellular calcium stores; wherein

prior to reduction, alteration or elimination of gene expression, the cell exhibits increases in cytosolic calcium levels after reduction of calcium levels of intracellular calcium stores.

Also provided is a method for identifying a protein involved in modulating intracellular calcium, comprising:

reducing, altering or eliminating expression of one or more genes in a cell; and

monitoring intracellular calcium level time courses of the resulting cell after reduction of calcium levels in an intracellular calcium store, whereby one or more proteins involved in modulating intracellular calcium and/or one or more nucleic acids encoding one or more proteins involved in modulating intracellular calcium is identified in cells in which a reduction, alteration or elimination of gene expression is associated with a reduction, alteration or elimination of the fluctuations in cytosolic calcium levels after reduction of calcium levels of intracellular calcium stores; wherein

prior to reduction, alteration or elimination of gene expression, the cell exhibits fluctuations in cytosolic calcium levels after reduction of calcium levels of intracellular calcium stores.

In particular embodiments of any of the above methods for identifying a protein involved in modulating intracellular calcium, the protein is an ion transport protein, calcium-binding protein, EF hand-containing protein and/or a presenilin loop-containing protein.

Further provided herein are any of the above methods wherein the cell is a Drosophila cell, such as, for example, an S2 cell.

Also provided herein are any of the above methods wherein the reduction, alteration or elimination of gene expression is achieved by introducing RNA into or generating RNA within the cell, wherein the RNA comprises a sequence of nucleotides complementary to or identical to at least a portion of a gene in the cell or an RNA transcript of a gene of the cell. The RNA may be double-stranded RNA.

Provided herein are any of the above methods, further comprising comparing the amino acid sequence of the identified protein or proteins to amino acid sequences of proteins of species different from the species from which the cell is obtained. In particular embodiments, the amino acid sequence of the identified protein or proteins is compared to mammalian protein amino acid sequences thereby identifying a mammalian protein involved in modulating intracellular calcium. The mammal may be human.

Provided herein are any of these methods, wherein the cell exhibits increases in intracellular calcium levels after contact with thapsigargin. Also provided are any of these methods, wherein the protein(s) identified as involved in intracellular calcium modulation is an ion transport protein.

Also provided herein are any of the above methods for identifying a protein involved in modulating intracellular calcium, wherein the one or more genes is (are) selected as targets for reduction, alteration or elimination of expression by a process comprising any one or more of the following steps:

(a) identifying one or more genes of the cell that encode(s) one or more proteins comprising six or more domains each of which comprises and/or has homology to an amino acid sequence characteristic of an amino acid sequence that extends through a cellular membrane;

(b) identifying one or more genes of the cell that encode(s) one or more proteins comprising one or more domains comprising and/or having homology to an amino acid sequence characteristic of a protein that transports a cation, such as, for example, calcium;

(c) identifying one or more genes of the cell that encode(s) one or more proteins comprising one or more domains containing and/or having homology to an amino acid sequence characteristic of a calcium-binding motif, e.g., an EF-hand; and

(d) identifying one or more genes of the cell that encode(s) one or more proteins comprising one or more domains containing and/or having homology to an amino acid sequence characteristic of a presenilin protein, e.g., a presenilin loop sequence.

In conducting this method, it is also possible to combine any of the above steps (a)-(d) by identifying genes according to one of the steps and then identifying from among those identified genes a subset of genes according to another of the steps. Thus, for example, the method may be conducted such that genes encoding proteins comprising one or more domains containing and/or having homology to an amino acid sequence characteristic of a protein that transports calcium are first identified and then, within that group of genes, a subset of genes is identified by evaluating those genes for encoded proteins that also comprise six or more domains each of which contains and/or has homology to an amino acid sequence characteristic of an amino acid sequence that extends through a cellular membrane. Similarly, the method may be conducted such that genes encoding proteins comprising one or more domains containing and/or having homology to an amino acid sequence characteristic of a protein that transports calcium are first identified and then, within that group of genes, a subset of genes is identified by evaluating those genes for encoded proteins that also comprise one or more domains containing and/or having homology to an amino acid sequence characteristic of a calcium-binding motif, and further, within that first subset of genes, a subset of genes is identified by evaluating the first subset of genes for encoded proteins that also comprise six or more domains each of which contains and/or has homology to an amino acid sequence characteristic of an amino acid sequence that extends through a cellular membrane. In another example, the method may be conducted such that genes encoding proteins comprising one or more domains containing and/or having homology to an amino acid sequence characteristic of a presenilin protein, e.g., a presenilin loop sequence, are first identified and then, within that group of genes, a subset of genes is identified by evaluating those genes for encoded proteins that also comprise six or more domains each of which contains and/or has homology to an amino acid sequence characteristic of an amino acid sequence that extends through a cellular membrane. In yet another example, the method may be conducted such that genes encoding proteins comprising one or more domains containing and/or having homology to an amino acid sequence characteristic of a calcium-binding motif, e.g., an EF-hand, are first identified and then, within that group of genes, a subset of genes is identified by evaluating those genes for encoded proteins that also comprise six or more domains each of which contains and/or has homology to an amino acid sequence characteristic of an amino acid sequence that extends through a cellular membrane.

F. Methods of Screening for or Identifying an Agent that Modulates Intracellular Calcium

Methods of screening for or identifying agents that modulate intracellular calcium are provided herein. The methods are based in directly or indirectly monitoring of the effect of test agents on intracellular calcium and/or ion movement. In particular embodiments, the methods involve monitoring of store-operated calcium entry, resting cytosolic calcium levels and/or calcium levels in (and/or ion flux into or out of) intracellular organelles, e.g., the endoplasmic reticulum. The effect(s) of test agents on intracellular calcium can be assessed in a variety of ways including, but not limited to, evaluation of calcium or other ion (particularly cation) levels, movement of calcium or other ion (particularly cation), fluctuations in calcium or other ion (particularly cation) levels, kinetics of calcium or other ion (particularly cation) fluxes and/or transport of calcium or other ion (particularly cation) through a membrane.

In one embodiment, the methods can be conducted using a particular test agent: one that binds to, interacts with and/or modulates interactions, activities, levels or any physical, structural or other property of a protein involved in modulating intracellular calcium. In this embodiment, the method includes monitoring the effects of such a particular test agent on intracellular calcium (and, in particular embodiments, the effects on store-operated calcium entry, intracellular organelle calcium storage, uptake or release and/or, resting cytosolic calcium levels).

In another embodiment, the methods can be performed by contacting any test agent with (1) one or more proteins involved in modulating intracellular calcium, e.g., an ion transport protein, and/or (2) a cell, or portion thereof, e.g., a membrane, containing one or more proteins involved in modulating intracellular calcium, such as an ion transport protein, and/or nucleic acid (e.g., a gene or coding sequence such as cDNA or RNA), or portion(s) thereof, encoding such proteins. The effect of the test agent on intracellular calcium (and, in particular embodiments, the effect on store-operated calcium entry, intracellular organelle calcium storage, uptake or release and/or, resting cytosolic calcium levels) is monitored or assessed. In this embodiment of the methods, the test agent can be any agent, and is not necessarily one that (or has been identified as one that) binds to, interacts with and/or modulates interactions, activities, levels or any physical, structural or other property of a protein involved in modulating intracellular calcium.

In particular embodiments of any of the methods of screening for or identifying an agent that modulates intracellular calcium provided herein, the protein involved in modulating intracellular calcium is a protein that is involved in, participates in and/or provides for store-operated calcium entry, maintenance of resting cytosolic calcium levels and/or calcium storage in, or movement of cations into or out of, an intracellular organelle, such as, for example, the endoplasmic reticulum. In particular embodiments, the protein involved in modulating intracellular calcium is an ion transport protein, such as, for example, an ion transport protein that is involved in, participates in and/or provides for store-operated calcium entry. In one embodiment, the protein involved in modulating intracellular calcium is a component of a store-operated calcium entry channel. The one or more proteins involved in modulating intracellular calcium (and/or nucleic acid, or portion(s) thereof, encoding one or more proteins involved in modulating intracellular calcium) may be contained in a cell or portion thereof, such as, for example, a cell membrane (e.g., plasma membrane or an intracellular membrane). The methods may be performed in particular embodiments under conditions that permit specific evaluation of store-operated ion flux or movement, resting cytosolic calcium levels and/or cation levels in, or movement into or out of, an intracellular organelle, such as, for example, the endoplasmic reticulum.

A protein involved in modulating intracellular calcium that is used in the methods can be a full-length or complete protein (e.g., a protein that contains the complete amino acid sequence encoded by a gene, cDNA or RNA or a complete amino acid sequence without sequences that are removed during processing of the protein, including removal of a signal sequence, such as may occur in transport of a protein to a particular cellular location, or processing to remove a pre- and/or pro-sequence of a protein) or a portion of a complete protein. In embodiments of the methods that involve assessing a functional activity of the protein, the protein used in the method can be a portion of a full-length protein that is associated with, or exhibits or is sufficient for producing the functional activity, e.g., an intracellular calcium-modulating activity. In embodiments of the methods that involve assessing a property of the protein that is not necessarily the intracellular calcium-modulating activity of the protein, the protein used in the method can be a portion of a full-length protein that is associated with the particular property being assessed, e.g., binding properties, and can be, for example, a particular domain of the protein. Similarly, when a nucleic acid, or portion(s) thereof, encoding a protein involved in modulating intracellular calcium is used in the methods, the nucleic acid can be a complete gene, e.g, including transcriptional regulatory sequences, a complete protein coding sequence, e.g., cDNA or RNA, or portion(s) of these.

1. Proteins (and/or Nucleic Acids Encoding Proteins) involved in Modulating Intracellular Calcium

The methods provided herein for screening for or identifying agents that modulate intracellular calcium are related to proteins (and/or nucleic acids, or portions thereof, encoding proteins) that are involved in modulating intracellular calcium. Some embodiments of the methods involve monitoring the effect of a test agent that binds to, interacts with and/or in some way modulates such proteins. Other embodiments involve the actual use of such a protein in a step of the method. For example, in these embodiments, a test agent can be contacted with (1) one or more proteins involved in modulating intracellular calcium, e.g., an ion transport protein, and/or (2) a cell, or portion thereof, e.g., a membrane, containing one or more proteins involved in modulating intracellular calcium, such as an ion transport protein, and/or nucleic acid (e.g., a gene or coding sequence such as cDNA or RNA), or portion(s) thereof, encoding such proteins.

Proteins (or nucleic acids, or portion(s) thereof, encoding proteins) used in the methods of screening for or identifying agents that modulate intracellular calcium provided herein (or proteins on which the methods are based) include proteins that are (or encode proteins that are) homologous to a protein encoded by a Drosophila gene that, when altered in its expression in a Drosophila cell, results in altered store-operated calcium entry into the cell, altered basal or resting cytosolic calcium levels and/or altered calcium levels in (and/or ion flux into or out of) intracellular organelles, e.g., the endoplasmic. An “alteration” of store-operated flux into a cellentry or calcium movement into, within or out of an intracellular organelle (e.g., the endoplasmic reticulum) can be a complete or nearly complete elimination of the activity, a reduction of the activity, an alteration in properties or characteristics of the activity (e.g., current properties or sensitivities) or an increase in the activity, e.g., relative to the activity in a control cell (e.g., a Drosophila cell) that has not been altered in its store-operated entry activity or calcium movement or storage activity with respect to an intracellular organelle. An “alteration” in the calcium level within an intracellular organelle or an alteration of resting cytosolic calcium levels can be, for example, a reduction, depletion, elimination of, or increase in calcium levels, e.g., relative to the levels in a control cell (e.g., a Drosophila cell) that has not been altered in its intracellular organelle or basal cytosolic calcium levels. Similarly, an “alteration” in gene expression may be complete or nearly complete elimination of the expression of a gene, a reduction in the expression of a gene, an increase in the expression of a gene, or an alteration in the protein encoded by the gene (such as truncation or other alteration that effectively renders the protein nonfunctional or provides for aberrant functioning of the protein), e.g., relative to the expression of the gene in a cell that has not been altered in its expression of the gene.

In particular embodiments of the methods for screening for or identifying agents that modulate intracellular calcium, a protein homologous to a protein encoded by a Drosophila gene that, when altered in its expression in a Drosophila cell, results in altered store-operated calcium entry into the cell is at least about 20%, or at least about 25%, or at least about 30%, or least about 35%, or at least about 40%, or at least about 45%, or at least about 50%, or at least about 52%, or at least about 55%, or at least about 60%, or at least about 65%, or at least about 70%, or at least about 75%, or at least about 80%, or at least about 85%, or at least about 90%, or at least about 95% or more homologous to the protein encoded by the coding sequence of the Drosophila gene. The particular homology can depend on the particular protein, e.g., species, that is homologous to the Drosophila gene, the extent of the encoded Drosophila protein to which the particular protein is homologous, and can also depend on the particular Drosophila gene that, when altered in its expression in a Drosophila cell, results in altered store-operated calcium entry into the cell. In particular embodiments, the protein is at least 35% or more homologous to the protein encoded by the coding sequence of the Drosophila gene. Such exemplary proteins may be homologous to the specified Drosophila protein over at least about 25%, or at least about 30%, or at least about 35%, or at least about 40%, or at least about 45%, or at least about 50%, or at least about 54%, or least about 55%, or at least about 60%, or at least about 65%, or at least about 70%, or at least about 75%, or at least about 80%, or at least about 85%, or at least about 90%, or at least about 95% or more of the protein encoded by the coding sequence of the Drosophila gene. In particular embodiments, the protein is homologous to the Drosophila protein over at least about 40% or more of the protein encoded by the coding sequence of the Drosophila gene.

In a particular embodiment of the above methods for screening for or identifying agents that modulate intracellular calcium, the protein (and/or protein encoded by a nucleic acid or portion(s) thereof used in the method) involved in modulating intracellular calcium is at least about 35% homologous over at least about 40% of the protein encoded by the coding sequence of the Drosophila gene.

In particular embodiments of the methods of screening for or identifying agents that modulate intracellular calcium provided herein, the protein used in the methods (and/or protein encoded by a nucleic acid or portion(s) thereof used in the methods) is an ion transport protein. In one embodiment, the protein can be involved in and/or provide for the movement of calcium and may be relatively specific for calcium ion transport. In one embodiment, the protein is an ion transport protein that is involved in, participates in and/or provides for store-operated calcium entry into cells.

In one embodiment of the methods of screening for or identifying agents that modulate intracellular calcium provided herein, the protein(s) used in the method (or the protein encoded by the nucleic acid, or portion(s) thereof, used in the method) contains six or more transmembrane domains. In another embodiment, the protein(s) used in the method (or the protein encoded by the nucleic acid, or portion(s) thereof, used in the method) does not contain the following sequence of amino acids: EWKFAR (SEQ ID NO: 114). This sequence of amino acids (EWKFAR) is a motif characteristic of transient receptor potential (trp) calcium channel proteins. In another embodiment, protein(s) used in the method (or the protein encoded by the nucleic acid, or portion(s) thereof, used in the method) does not contain the following sequence of amino acids (wherein “X” represents any amino acid, and an amino acid residue in parentheses is an alternative to the residue immediately preceeding it): EXD(E)CR(K)GXYXXYE (SEQ ID NO: 1115). This sequence of amino acids [EXD(E)CR(K)GXYXXYE] is a motif characteristic of a pore-forming region of calcium channels such as voltage-gated calcium channels. In another embodiment, the protein(s) used in the method (or the protein encoded by the nucleic acid, or portion(s) thereof, used in the method) does not contain either of the above-mentioned amino acid sequences, i.e., EWKFAR or EXD(E)CR(K)GXYXXYE.

In a particular embodiment of the methods of screening for or identifying agents that modulate intracellular calcium provided herein, the protein used in the methods (and/or protein encoded by a nucleic acid or portion(s) thereof used in the methods) is involved in modulating intracellular calcium (and, in particular embodiments, is involved in, participates in and/or provides for store-operated calcium entry, intracellular organelle calcium storage, uptake or release and/or, maintenance of resting cytosolic calcium levels) and is homologous to a protein encoded by the coding sequence of Drosophila gene CG4536 (see SEQ ID NO: 1 for a nucleotide coding sequence and SEQ ID NO:2 for amino acid sequence) and/or the the protein encoded by the coding sequence of Drosophila gene CG5842 (see SEQ ID NO: 3 for a nucleotide coding sequence and SEQ ID NO: 4 for amino acid sequence). In a further embodiment, the protein used in the method is an ion transport protein.

In a particular embodiment of the methods of screening for or identifying agents that modulate intracellular calcium, a protein used in the method (or a protein encoded by nucleic acid, or portion(s) thereof, used in the method) is one that is involved in modulating intracellular calcium (and, in particular embodiments, is involved in, participates in and/or provides for store-operated calcium entry, intracellular organelle calcium storage, uptake or release and/or, maintenance of resting cytosolic calcium levels) and that has an amino acid sequence that is at least about 20% or at least about 25% or at least about 30% or at least about 35% or at least about 40% or at least about 45% or at least about 46% or at least about 47%, or at least about 48%, or at least about 49%, or at least about 50%, or least about 52%, or at least about 55%, or at least about 60%, or at least about 65%, or at least about 70%, or at least about 75%, or at least about 80%, or at least about 85%, or at least about 90%, or at least about 95% or more homologous to an amino acid sequence of the protein encoded by the coding sequence of Drosophila gene CG4536 (e.g., Genbank Accession No. AAF46203; gi7290757 or SEQ ID NO: 2) or gene CG5842 (e.g., Genbank Accession No. AAF49752; gi7294406 or SEQ ID NO: 4). The particular homology can depend on the particular protein, e.g., species, that is homologous to a protein encoded by Drosophila gene CG4536 and/or CG5842 and the extent of the protein encoded by the Drosophila genes to which the particular protein is homologous. In particular embodiments, the protein is at least about 35%, or at least about 43% or at least about 44%, or at least about 45%, or at least about 46%, or at least about 47%, or at least about 48% or at least about 50% homologous to one or both of the proteins encoded by Drosophila genes CG4536 and CG5842. Such exemplary proteins may be homologous to the specified Drosophila proteins over at least about 25%, or at least about 27%, or at least about 30%, or at least about 35%, or at least about 40%, or at least about 43%, or at least about 45%, or at least about 50%, or at least about 51%, or at least about 52%, or at least about 53%, or at least about 54% or at least about 55%, or at least about 60%, or at least about 65%, or at least about 70%, or at least about 75%, or at least about 80%, or at least about 85%, or at least about 90%, or at least about 95% or more of the protein encoded by the coding sequence of Drosophila gene CG4536 (e.g., Genbank Accession No. AAF46203; gi7290757 or SEQ ID NO: 2) and/or CG5842 (Genbank Accession No. AAF49752; gi7294406 or SEQ ID NO: 4). In particular embodiments, the protein is homologous to the Drosophila protein over at least about 25%, or at least about 40%, or at least about 43%, or at least about 45%, or at least about 50%, or at least about 51%, or at least about 52% or more of the protein encoded by the coding sequence of Drosophila gene CG4536 and/or CG5842.

In a particular embodiment of the methods of screening for or identifying agents that modulate intracellular calcium, a protein used in the method (or proteins encoded by nucleic acids, or portion(s) thereof, used in the method) is one that is involved in modulating intracellular calcium (and, in particular embodiments, is involved in, participates in and/or provides for store-operated calcium entry, intracellular organelle calcium storage, uptake or release and/or, maintenance of resting cytosolic calcium levels) and that has an amino acid sequence that is at least about 35% homologous over at least about 40% of the protein encoded by the coding sequence of Drosophila gene CG4536 (and/or CG5842), or at least about 35% homologous over at least about 50% or 51% or 52% of the protein encoded by the coding sequence of Drosophila gene CG4536 (and/or CG5842), or at least about 41% homologous over at least about 50% or 51% or 52% of the protein encoded by the coding sequence of Drosophila gene CG4536 (and/or CG5842).

Proteins homologous to a protein encoded by the coding sequence of Drosophila gene CG4536 and/or CG5842 include, but are not limited to: calcium transport protein 1 (CaT1), epithelial calcium channel (ECaC;, including proteins referred to as CaT2 proteins), olfactory channels, stretch-activated channel proteins, vanilloid receptor-related osmotically activated channel proteins, vanilloid receptor type 1-like proteins, vanilloid receptor subtype 1 proteins, vanilloid receptor 1 proteins, vanilloid receptor-like proteins, capsaicin receptor proteins, OTRPC4 proteins, growth factor-regulated calcium channel proteins, transient receptor potential protein 12 (trp12), transient receptor potential protein V3 (TRPV3) and stretch-inhibitable nonselective channel (SIC) proteins.

Calcium Transport 1 (CaT1) Proteins

In a particular embodiment of the methods of screening for or identifying agents that modulate intracellular calcium, the protein used in the method (or proteins encoded by nucleic acids, or portion(s) thereof, used in the method) is a Calcium Transport Protein 1 (CaT1). CaT1 proteins are ion transport proteins that selectively transport calcium and display the predicted six-transmembrane domain architecture characteristic of a channel-forming protein [see, e.g., Peng et al. (1999) J. Biol. Chem. 274:22739-22746; Peng et al. (2000) Biochem. Biophys. Res. Commun. 278:326-332]. CaT1 proteins participate in transepithelial calcium transport and can mediate cellular calcium uptake as part of the transcellular pathway of calcium transport in intestine, kidney and placenta. In this respect, CaT1 channels can function as facilitated transporters that enable calcium to move down its electrochemical gradient from the external medium to the internal medium of renal and intestinal cells. Other tissues in which CaT1 is expressed include prostate, pancreas, salivary gland, liver and testis.

Exemplary CaT1 proteins include the following: a Homo sapiens calcium transport protein CaT1 (GenBank accession no. AAG41951 (SEQ ID NO: 6; see also SEQ ID NO: 5 for nucleic acid encoding the protein); having 27% identity and 45% homology over 52% of the Drosophila gene CG4536 protein); a Homo sapiens Cat-like B protein (GenBank accession no. CAC20417 (SEQ ID NO: 8; see also SEQ ID NO: 7 for nucleic acid encoding the protein); having 27% identity and 45% homology over 52% of the Drosophila gene CG4536 protein); a Homo sapiens Cat-like A protein (GenBank accession no. CAC20416 (SEQ ID NO: 10; see also SEQ ID NO: 9 for nucleic acid encoding the protein); having 27% identity and 44% homology over 52% of the Drosophila gene CG4536 protein); a Rattus norvegicus calcium transporter CaT1 protein (GenBank accession no. AAD47636 (SEQ ID NO: 12; see also SEQ ID NO: 11 for nucleic acid encoding the protein); having 26% identity and 44% homology over 51% of the Drosophila gene CG4536 protein); a Mus musculus calcium transporting protein homolog (GenBank accession no. BAA99538 (SEQ ID NO: 14; see also SEQ ID NO: 13 for nucleic acid encoding the protein); having 24% identity and 41% homology over 51% of the Drosophila gene CG4536 protein); a Homo sapiens calcium transport protein CaT1 (GenBank accession no. AAL40230 (SEQ ID NO: 16; see also SEQ ID NO: 15 for nucleic acid encoding the protein); having 27% identity and 45% homology over 52% of the Drosophila gene CG4536 protein); a Mus musculus protein similar to epithelial apical membrane calcium transporter/channel CaT1 (GenBank accession no. AAH16101 (SEQ ID NO: 18; see also SEQ ID NO: 17 for nucleic acid encoding the protein); having 27% identity and 44% homology over 51% of the Drosophila gene CG4536 protein); and the like. Particular CaT1 proteins that may be used in the methods provided herein include, but are not limited to, CaT1 proteins that are substantially homologous (including, e.g., at least about 98%, 95%, 90%, 85% or 80% homologous) to the above-listed proteins.

Epithelial Calcium Channel Proteins

Epithelial calcium channel (ECaC) proteins (also referred to as CaT2 proteins) are ion transport proteins that facilitate transepithelial calcium transport and apical calcium uptake in intestine and kidney. These proteins are also thought to participate in calcium entry into the cells of the distal convoluted tubule and connecting segment of the nephron. They also possess a high sensitivity to Cd²⁺ (see, e.g., Peng et al., (2000) J. Biol. Chem., 275(36):28186-28194; Muller et al., (2000) Genomics, 67:48-53). Other tissues in which ECaC is expressed include placenta, pancreas, brain, adrenal gland, heart, skeletal muscle, liver, lung, spleen, thymus, and testis.

Exemplary ECaC proteins include the following: a Oryctolagus cuniculus epithelial calcium channel (GenBank accession no. CAB40138 (SEQ ID NO: 20; see also SEQ ID NO: 19 for nucleic acid encoding the protein); having 25% identity and 44% homology over 51% of the Drosophila gene CG4536 protein); a Rattus norvegicus epithelial calcium channel (GenBank accession no. BAA99541 (SEQ ID NO: 22; see also SEQ ID NO: 21 for nucleic acid encoding the protein); having 26% identity and 43% homology over 51% of the Drosophila gene CG4536 protein); a Homo sapiens epithelial calcium channel (GenBank accession no. CAB96365 (SEQ ID NO: 24; see also SEQ ID NO: 23 for nucleic acid encoding the protein); having 25% identity and 42% homology over 51% of the Drosophila gene CG4536 protein); a Rattus norvegicus calcium transporter CaT2 protein (GenBank accession no. AAF86309 (SEQ ID NO: 26; see also SEQ ID NO: 25 for nucleic acid encoding the protein); having 26% identity and 43% homology over 51% of the Drosophila gene CG4536 protein); a Homo sapiens protein with similarity to calcium transport protein CaT2 (GenBank accession no. XP 005003 (SEQ ID NO: 28 see also SEQ ID NO: 27 for nucleic acid encoding the protein); having 25% identity and 43% homology over 42% of the Drosophila gene CG4536 protein); a Homo sapiens calcium transport protein CaT2 (GenBank accession no. AAL04015 (SEQ ID NO: 30 see also SEQ ID NO: 29 for nucleic acid encoding the protein); having 25% identity and 43% homology over 51% of the Drosophila gene CG4536 protein); and the like. Particular ECaC proteins that may be used in the methods provided herein include, but are not limited to, ECaC proteins that are substantially homologous (including, e.g., at least about 98%, 95%, 90%, 85% or 80% homologous) to the above-listed proteins.

Olfactory Channel Proteins

Olfactory channel proteins display the predicted six-transmembrane domain architecture characteristic of a channel-forming protein. These proteins are expressed in sensory neurons that mediate chemosensory, osmosensory, and mechanosensory functions (Colbert et al. (1997) J. Neurosci. 17(21):8259-69). An exemplary olfactory channel protein includes, but is not limited to the olfactory channel of C. elegans (GenBank accession no. AAB87064 (SEQ ID NO: 32; see also SEQ ID NO: 31 for nucleic acid encoding the protein); having 42% identity and 61% homology over 65% of Drosophila gene CG4536 protein). Particular Olfactory channel proteins that may be used in the methods provided herein include, but are not limited to, olfactory proteins that are substantially homologous (including, e.g., at least about 98%, 95%, 90%, 85% or 80% homologous) to the above-listed protein.

Stretch-Activated Channel Proteins

Stretch-activated channel (SAC) proteins or mechanosensitive channels (MSC) proteins are expressed in a wide variety of cell types (kidney, heart, etc.) and have been implicated in diverse functions. Some of which include osmoregulation and mechanoreception or mechanotransduction (membrane tension or deformation leads to channel activity). Stretch-activated cation channels have been suggested to act as endothelial mechanosensors for hemodynamic forces.[see Brakemeier et al. (2002) Cardiovas. Res. 53:209-218]. An exemplary SAC protein includes, but is not limited to the the stretch-activated channel 2B protein of Rattus norvegicus (GenBank accession no. BAA88637 (SEQ ID NO: 34; see also SEQ ID NO: 33 for nucleic acid encoding the protein); having 25% identity and 40% homology over 52% of the Drosophila gene CG4536 protein). Particular SAC proteins that may be used in the methods provided herein include, but are not limited to, SAC proteins that are substantially homologous (including, e.g., at least about 98%, 95%, 90%, 85% or 80% homologous) to the above-listed protein.

Vanilloid Receptor-Related Proteins

Vanilloid receptor-related proteins (e.g., vanilloid receptor-related osmotically activated channel proteins; vanilloid receptor type 1-like proteins; vanilloid receptor subtype 1 proteins; vanilloid receptor 1 proteins; vanilloid receptor-like proteins; transient receptor potential protein 12 (trp12) proteins; OTRPC4 proteins; capsaicin receptors; and the like) are are known to be expressed by sensory neurons. Once activated, these neurons evoke the sensation of burning pain and stimulate the release of neuropeptides that induce neurogenic inflammation. VR1 immunoreactivity is greatly increased in colonic nerve fibres of patients with active inflammatory bowel disease (Yiangou et al (2001) Lancet (North American Edition) 357: 1338-1339). VR1 receptors have also been shown to play a role in airway inflammation (Veronesi et al. (2000) Toxicology and Applied Pharmacology 169: 66-76) and may, thus, be important in the treatment of chronic obstructive pulmonary disease.

The cloned capsaicin receptor (also referred to as vanilloid receptor 1, VR1) reportedly is activated by increases in temperature in the noxious range, suggesting that it functions as a transducer of painful thermal stimuli in vivo. Because a vanilloid moiety constitutes an essential chemical component of capsaicin and resiniferatoxin structures, the proposed site of action of these compounds is generally referred to as the vanilloid receptor. Accordingly, Caterina et al. [(1997) Nature 389:816-824] named their cloned cDNA VR1, for “vanilloid receptor subtype 1.” An expressed sequence tag (EST) database search revealed several human clones with a high degree of similarity to VR1 at both the DNA and predicted amino acid sequence levels. VR1 and native VRs are nonselective cation channels reportedly directly activated by harmful heat, extracellular protons, and vanilloid compounds. VR1 is also expressed in nonsensory tissue and may mediate inflammatory rather than acute thermal pain. Premkumar and Ahern [(2000) Nature 408:985-990] showed that activation of PKC-epsilon induces VR1 channel activity at room temperature in the absence of any other agonist. They also observed this effect in native VRs from sensory neurons, and phorbol esters induced a vanilloid-sensitive calcium rise in these cells. The proinflammatory peptide bradykinin, and the putative endogenous ligand anandamide, induced and enhanced VR activity, respectively, in a PKC-dependent manner.

Exemplary vanilloid receptor-related proteins include the following:

the vanilloid receptor-related osmotically activated channel protein of Gallus gallus (Genbank accession no. AAG28026 (SEQ ID NO: 36; see also SEQ ID NO: 35 for nucleic acid encoding the protein); having 24% identity and 42% homology over 54% of the Drosophila gene CG4536 protein);

the vanilloid receptor type 1 like protein 1 of Rattus norvegicus (Genbank accession no. BAA94307 (SEQ ID NO: 38; see also SEQ ID NO: 37 for nucleic acid encoding the protein); having 24% identity and 39% homology over 57% of the Drosophila gene CG4536 protein);

the vanilloid receptor subtype 1 of Rattus norvegicus (GenBank accession no. AAC53398 (SEQ ID NO: 40; see also SEQ ID NO: 39 for nucleic acid encoding the protein); having 24% identity and 39% homology over 57% of the Drosophila gene CG4536 protein);

the vanilloid receptor 1 of Homo sapiens (GenBank accession no. CAB95729 (SEQ ID NO: 42; see also SEQ ID NO: 41 for nucleic acid encoding the protein); having 24% identity and 40% homology over 57% of the Drosophila gene CG4536 protein);

the vanilloid receptor-like protein 1 of Homo sapiens (GenBank accession no. XP_{—}054949 (SEQ ID NO: 44; see also SEQ ID NO: 43 for nucleic acid encoding the protein); having 24% identity and 39% homology over 50% of the Drosophila gene CG4536 protein);

the vanilloid receptor-like protein 1 of Rattus norvegicus (GenBank accession no. AAD26364 (SEQ ID NO: 46; see also SEQ ID NO: 45 for nucleic acid encoding the protein); having 25% identity and 40% homology over 52% of the Drosophila gene CG4536 protein);

the vanilloid receptor subtype 1 of Homo sapiens (GenBank accession no. XP 008512 (SEQ ID NO: 48; see also SEQ ID NO: 47 for nucleic acid encoding the protein); having 23% identity and 39% homology over 57% of the Drosophila gene CG4536 protein); the vanilloid receptor 1 of Homo sapiens (GenBank accession no.

CAB89866 (SEQ ID NO: 50; see also SEQ ID NO: 49 for nucleic acid encoding the protein); having 24% identity and 40% homology over 57% of the Drosophila gene CG4536 protein);

the vanilloid receptor-like protein 1 of Mus musculus (GenBank accession no. AAH05415 (SEQ ID NO: 52; see also SEQ ID NO: 51 for nucleic acid encoding the protein); having 25% identity and 40% homology over 50% of the Drosophila gene CG4536 protein);

a protein of Homo sapiens similar to vanilloid receptor-related osmotically activated channel (GenBank accession no. XP_{—}027181 (SEQ ID NO: 54; see also SEQ ID NO: 53 for nucleic acid encoding the protein); having 24% identity and 42% homology over 54% of the Drosophila gene CG4536 protein);

the vanilloid receptor-like protein 2 of Homo sapiens (GenBank accession no. AAK69487 (SEQ ID NO: 56; see also SEQ ID NO: 55 for nucleic acid encoding the protein); having 24% identity and 42% homology over 52% of the Drosophila gene CG4536 protein);

the vanilloid receptor-like channel 2 protein of Homo sapiens (GenBank accession no. BAB69040 (SEQ ID NO: 58; see also SEQ ID NO: 57 for nucleic acid encoding the protein); having 24% identity and 42% homology over 52% of the Drosophila gene CG4536 protein);

the vanilloid receptor-related osmotically activated channel of Homo sapiens (GenBank accession No. AAG28029 (SEQ ID NO: 60; see also SEQ ID NO: 59 for nucleic acid encoding the protein); having 24% identity and 42% homology over 54% of the Drosophila gene CG4536 protein);

the vanilloid receptor-like protein 2 of Mus musculus (GenBank accession no. AAK69486 (SEQ ID NO: 62; see also SEQ ID NO: 61 for nucleic acid encoding the protein); having 24% identity and 41% homology over 52% of the Drosophila gene CG4536 protein);

the vanilloid receptor-related osmotically activated channel of Mus musculus (GenBank accession no. AAG28028 (SEQ ID NO: 64; see also SEQ ID NO: 63 for nucleic acid encoding the protein); having 24% identity and 41% homology over 52% of the Drosophila gene CG4536 protein);

the vanilloid receptor-related osmotically activated channel of Rattus norvegicus (GenBank accession No. AAG28027 (SEQ ID NO: 66; see also SEQ ID NO: 65 for nucleic acid encoding the protein); having 24% identity and 42% homology over 52% of the Drosophila gene CG4536 protein);

the vanilloid receptor-like protein 1 of Homo sapiens (GenBank accession No. AAD26363 (SEQ ID NO: 68; see also SEQ ID NO: 67 for nucleic acid encoding the protein); having 24% identity and 39% homology over 50% of the Drosophila gene CG4536 protein);

the vanilloid receptor-like protein of Homo sapiens (GenBank accession no. AAD41724 (SEQ ID NO: 70; see also SEQ ID NO: 69 for nucleic acid encoding the protein); having 24% identity and 39% homology over 50% of the Drosophila gene CG4536 protein);

the vanilloid receptor type 1 like protein 2 of Rattus norvegicus (GenBank accession no. BAA94306 (SEQ ID NO: 72; see also SEQ ID NO: 71 for nucleic acid encoding the protein); having 22% identity and 36% homology over 56% of the Drosophila gene CG4536 protein);

a protein of Homo sapiens described by Wiemann et al. [(2001) Genome Res. 11: 422-35], having a strong sequence similarity to rat capsaicin/vanilloid receptor subtype 1 (GenBank accession no. CAB66735 (SEQ ID NO: 74; see also SEQ ID NO: 73 for nucleic acid encoding the protein); having 23% identity and 39% homology over 57% of the Drosophila gene CG4536 protein);

the vanilloid receptor-related osmotically activated channel of Homo sapiens (GenBank accession no. XP 012261 (SEQ ID NO: 76; see also SEQ ID NO: 75 for nucleic acid encoding the protein); having 24% identity and 42% homology over 54% of the Drosophila gene CG4536 protein);

the OTRPC4 protein described by Strotmann et al. [(2000) Nature Cell Biol. 2: 695-702] (GenBank accession no. AAG16127 (SEQ ID NO: 78; see also SEQ ID NO: 77 for nucleic acid encoding the protein); having 24% identity and 42% homology over 52% of the Drosophila gene CG4536 protein);

the OTRPC4 cation channel of Mus musculus (GenBank accession no. AAG17543 (SEQ ID NO: 80; see also SEQ ID NO: 79 for nucleic acid encoding the protein); having 24% identity and 41% homology over 52% of the Drosophila gene CG4536 protein);

the capsaicin receptor of Homo sapiens (GenBank accession No. AAG43466 (SEQ ID NO: 82; see also SEQ ID NO: 81 for nucleic acid encoding the protein); having 23% identity and 39% homology over 57% of the Drosophila gene CG4536 protein);

the transient receptor potential protein 12 (trp 12) from the Mus musculus kidney (GenBank accession no. CAC20703 (SEQ ID NO: 84; see also SEQ ID NO: 83 for nucleic acid encoding the protein); having 24% identity and 41% homology over 52% of the Drosophila gene CG4536 protein); and the like. Particular vanilloid receptor-related proteins that may be used in the methods provided herein include, but are not limited to, vanilloid receptor-related proteins that are substantially homologous (including, e.g., at least about 98%, 95%, 90%, 85% or 80% homologous) to the above-listed proteins.

Growth Factor-Regulated Channel Proteins

Growth factor-regulated calcium (GRCs) channel proteins are calcium-permeable cation channels that are reportedly regulated by growth factors, such as insulin-like growth factor-I (IGF-I), and the like. These particular channels localize mainly to intracellular pools under basal conditions. Upon stimulation of cells by IGF-I, the GRC translocates to the plasma membrane. Thus, IGF-I augments calcium entry through GRCs by regulating trafficking of the channel. An exemplary Growth factor-regulated calcium channel protein includes, but is not limited to the growth factor regulated calcium channel of Mus musculus (GenBank accession No. BAA78478 (SEQ ID NO: 86; see also SEQ ID NO: 85 for nucleic acid encoding the protein); having 25% identity and 40% homology over 50% of the Drosophila gene CG4536 protein); and the like. Particular Growth factor-regulated calcium channel proteins that may be used in the methods provided herein include, but are not limited to, Growth factor-regulated calcium channel proteins that are substantially homologous (including, e.g., at least about 98%, 95%, 90%, 85% or 80% homologous) to the above-listed protein.

Stretch-Inhibitable Channel Proteins

Stretch-inhibitable nonselective channel (SIC) proteins are mechanosensitive and are homologs of the capsaicin receptor-nonselective cation channels. The SIC channel can be inactivated by membrane stretch. The mechanosensitive nonselective channels have been widely investigated and are considered to play important roles in the control of various cell functions, including cell volume regulation, smooth muscle contraction, and cardiac rhythm generation. There are two groups of mechanosensitive nonselective channels: stretch activated and stretch inactivated. Even though mechanosensitive nonselective channels pass only monovalent cations under physiological conditions, activation of them elicits membrane depolarization. Thus, mechanosensitive nonselective channels may alter membrane potential or transport cations in response to cell volume changes or stretching of the cell membrane. One known isoform of SIC encodes a 563-amino acid protein with putative six transmembrane segments (see Suzuki et al. (1999) J. Biol. Chem. 274:6330-6335). The cDNA was expressed in mammalian cells, and electophysiological studies were performed. SIC-induced large cation currents were found to be regulated by cell volume, with currents being stimulated by cell shrinkage and inhibited by cell swelling.

An exemplary SIC protein includes, but is not limited to the stretch-inhibitable nonselective channel (SIC) of Rattus norvegicus (GenBank accession No. BAA34942 (SEQ ID NO: 88; see also SEQ ID NO: 87 for nucleic acid encoding the protein); having 25% identity and 40% homology over 40% of the Drosophila gene CG4536 protein); and the like. Particular SIC proteins that may be used in the methods provided herein include, but are not limited to, SIC proteins that are substantially homologous (including, e.g., at least about 98%, 95%, 90%, 85% or 80% homologous) to the above-listed protein.

Transient Receptor Potential V3 (TRPV3) Proteins Transient Receptor Potential V3 (TRPV3 or VRL3) are thermosensitive channels expressed in skin cells (keratinocytes) [see, e.g., Peier et al. (2002) Science 296:2046-2049] and dorsal root ganglion, trigeminal ganglion, spinal cord and brain [see Xu et al. (2002) Nature 418:181-185; Smith et al. (2002) Nature 418:186-188]. Particular TRPV3 proteins that may be used in the methods provided herein include, but are not limited to, Homo sapiens TRPV3 cation channel (GenBank accession No.AAM54027 (SEQ ID NO: 107; see also SEQ ID NO: 106 for nucleic acid encoding the protein); having 25% identity and 41% homology over 49% of the Drosophila gene CG4536 protein); and Mus musculus TRPV3 ion channel (GenBank accession No.AAM33069 (SEQ ID NO: 109; see also SEQ ID NO: 108 for nucleic acid encoding the protein); having 26% identity and 42% homology over 49% of the Drosophila gene CG4536 protein). TRPV3 proteins that are substantially homologous (including, e.g., at least about 98%, 95%, 90%, 85% or 80% homologous) to the above-listed proteins may also be used in the methods.

Other proteins contemplated for use in the methods provided herein include: the C. elegans protein having similarity to Pfam domain: PF00023 (GenBank accession no. AAF60537 (SEQ ID NO: 90; see also SEQ ID NO: 89 for nucleic acid encoding the protein); having 29% identity and 48% homology over 63% of the Drosophila gene CG4536 protein); the C. elegans protein GenBank accession no. AAC04431 (SEQ ID NO: 92; see also SEQ ID NO: 91 for nucleic acid encoding the protein) having 25% identity and 43% homology over 77% of the Drosophila gene CG4536 protein; the C. elegans protein having similarity to Pfam domain: PF00023 (GenBank accession no. CAA98449 (SEQ ID NO: 94; see also SEQ ID NO: 93 for nucleic acid encoding the protein); having 26% identity and 43% homology over 66% of the Drosophila gene CG4536 protein); the Homo sapiens protein having similarity to human ankyrin (GenBank accession no. CAA96679 (SEQ ID NO: 96; see also SEQ ID NO: 95 for nucleic acid encoding the protein); having 29% identity and 46% homology over 40% of the Drosophila gene CG4536 protein); the Rattus norvegicus ion channel (GenBank accession no. BAA93435 (SEQ ID NO: 98; see also SEQ ID NO: 97 for nucleic acid encoding the protein); having 25% identity and 40% homology over 52% of the Drosophila gene CG4536 protein); Anopheles gambiae protein ebiP7471 (GenBank accession no. EAA06511 (SEQ ID NO: 111; see also SEQ ID NO: 110 for nucleic acid encoding the protein); having 61% identity and 70% homology over 100% of the Drosophila gene CG4536 protein); and Anopheles gambiae protein ebiP9093 (GenBank accession no. EAA00229 (SEQ ID NO: 113; see also SEQ ID NO: 112 for nucleic acid encoding the protein); having 30% identity and 47% homology over 63% of the Drosophila gene CG4536 protein). In other embodiments, other proteins that may be used in the methods provided herein include, but are not limited to, proteins that are substantially homologous (including, e.g., at least about 98%, 95%, 90%, 85% or 80% homologous) to the above-listed protein.

These listed proteins and their sequences exemplify proteins that are useful for methods, materials and systems of the invention. Naturally occurring and synthesized alternative forms of these proteins such as allelic forms, isoforms, muteins, and mutated derivatives will have sequence changes that do not abolish their operation in modulating intracellular calcium and are useful in embodiments. Many mutations and allelic forms of these proteins are known and are contemplated as embodiments. In yet another embodiment one or more of the proteins associates with a presenilin protein and forms a calcium channel that may be controlled through binding interactions with or mediated by the presenilin protein.

Proteins used in the methods for screening for or identifying an agent that modulates intracellular calcium include synthetic proteins, proteins endogenously expressed by a cell, and recombinant proteins that are isolated from a source containing the protein or that are expressed by a cell, virus or organism. Nucleic acids used in the methods include synthetic nucleic acids, recombinant nucleic acids and nucleic acids isolated from a source containing the nucleic acid.

2. Test Agents

Generally, agents tested in the methods of screening for or identifying agents that modulate intracellular calcium provided herein can be of any physical type. Examples of agents include, but are not limited to, biomolecules, including, but not limited to, amino acids, peptides, polypeptides, peptiomimetics, nucleotides, nucleic acids (including DNA, cDNA, RNA, antisense RNA and any double- or single-stranded forms of nucleic acids and derivatives and structural analogs thereof), polynucleotides, saccharides, fatty acids, steroids, carbohydrates, lipids, lipoproteins and glycoproteins. Such biomolecules can be substantially purified, or can be present in a mixture, such as a cell extract or supernate. Test agents further include synthetic or natural chemical compounds, such as simple or complex organic molecules, metal-containing compounds and inorganic ions (including, for example, Gd³⁺, lead and lanthinum). Also included are pharmacological compounds, which optionally can be subjected to directed or random chemical modifications, such as acylation, alkylation, esterification, amidation, etc., to produce structural analogs.

Test agents suitable for use in the methods can optionally be contained in compound libraries. Methods for producing compound libraries by random or directed synthesis of a wide variety of organic compounds and biomolecules are known in the art, and include expression of randomized oligonucleotides and oligopeptides. Methods of producing natural compounds in the form of bacterial, fungal, plant and animal extracts are also known in the art. Additionally, synthetically produced or natural compounds and compound libraries can be readily modified through conventional chemical, physical and biochemical means to produce combinatorial libraries. Compound libraries are also available from commercial sources.

3. Methods Based on Testing an Agent that Binds to, Interacts with and/or Modulates Interactions, Activities or Levels of a Protein Involved in Modulating Intracellular Calcium (and/or Nucleic Acid, or Portion(S) thereof, Encoding Such Proteins)

In one embodiment of the methods for screening for or identifying agents that modulate intracellular calcium provided herein, the test agent is one that binds to, interacts with and/or modulates interactions, activities, levels or any physical, structural or other property of a protein (e.g., an ion transport protein) involved in modulating intracellular calcium. Such particular test agents include agents that are known to (or that are identified as agents that can) bind to, interact with and/or modulate interactions, activities, levels or any physical, structural or other property of a protein involved in modulating intracellular calcium. Thus, this embodiment of the methods includes a step of assessing or monitoring the effects of such a test agent on intracellular calcium (and, in particular embodiments, the effects on store-operated calcium entry, intracellular organelle calcium storage, uptake or release and/or, resting cytosolic calcium levels).

The protein involved in modulating intracellular calcium that the test agent binds to, interacts with and/or modulates (e.g., the interactions, activities, levels or any physical, structural or other property of the protein) can be proteins as provided herein above. In particular embodiments, the protein is one that is involved in, participates in, and/or provides for store-operated calcium entry, maintenance of resting cytosolic calcium levels and/or calcium storage in, or movement of cations into or out of, an intracellular organelle or compartment (e.g., endoplasmic reticulum). In one embodiment of the methods, the effect of test agent on store-operated calcium entry is monitored.

a. Identification of Particular Test Agents

Test agents suitable for use in this embodiment of the methods for screening for or identifying agents that modulate intracellular calcium can be identified in a number of ways using methods described herein and/or known in the art.

Binding and Interaction Assays

A number of in vitro and cell-based binding assays are known in the art and can be modified as needed by one of skill in the art to identify agents that bind to or interact with a protein involved in modulating intracellular calcium. Test agents that bind to a protein involved in modulating intracellular calcium include molecules that physically interact with the protein with relatively high affinity and selectivity. For example, an agent that binds to a protein involved in modulating intracellular calcium can bind with a Kd of about 10⁻⁴ M or less, such as about 10⁻⁶ M or less, including about 10⁻⁸ M or about 10⁻⁹ M or less. In contrast, under the same conditions, the agent can bind a protein that is not the particular protein involved in modulating intracelluar calcium with an affinity that is at least 10-fold lower, such as at least 100-fold or 1000-fold lower.

In vitro methods for identifying molecules that bind to or interact with a protein involved in modulating intracellular calcium include both direct methods, e.g., in which binding between the agent and the polypeptide is measured, and competitive methods, in which the ability of an agent to displace binding between a bound molecule and the polypeptide is measured.

Exemplary in vitro methods include co-purification assays (e.g., GST pull-down assays, co-immunoprecipitation assay, chromatographic assays), phage display (see, e.g., Rodi et al. (2002) Curr. Opin. Chem. Biol. 6:92-96), ribozyme display (Hanes and Pluckthun (1997) Proc. Natl. Acad. Sci. U.S.A. 13:4937-4942), and protein arrays (Cahill (2001) J. Immunol. Meth. 250:81-91).

Detection of in vitro binding or interaction between an agent and a protein involved in modulating intracellular calcium can involve a variety of approaches, such as, for example, nuclear magnetic resonance (NMR) (Hadjuk et al. (1999) J. Med. Chem. 42:2315-2317), mass spectroscopy (Siegel (2002) Curr. Top. Med. Chem. 2:13-33), fluorescence spectroscopy (Winkler et al. (1999) Proc. Natl. Acad. Sci. U.S.A. 96:1375-01378), scintillation proximity assays (SPQ) (Fernandes (1998) Curr. Opin. Chem. Biol. 2:597-603), surface plasmon resonance assays (available commercially from BIACORE; http://www.biacore.se/proteomics/), and others. Many of these methods are amenable to high-throughput screening of test agents.

The protein involved in modulating intracellular calcium, the agent, or both, used in an in vitro binding or interaction assay can be in gas phase, in solution, in suspension, or attached to a solid support, as appropriate for the assay method. The protein, agent, or both can be detectably labeled. Methods for preparing the protein or test agent in a form suitable for the particular assay are known in the art.

Cell-based binding assays include, for example, yeast two-hybrid assays (see, e.g., U.S. Pat. Nos. 5,283,173, 5,468,614 and 5,667,973); bacterial two-hybrid assays (Juong (2001) J. Cell Biochem. Suppl. 37:53-57), and others. Such assays are particularly suitable for identifying polypeptides that interact with a protein involved in modulating intracellular calcium. Two-hybrid assays are based on the modular nature of most transcription factors, which consist of separable DNA-binding and activation domains. Briefly, such assays use two different DNA constructs, one that codes for the polypeptide of interest (i.e., a protein involved in modulating intracellular calcium or portion thereof) fused to a gene or nucleic acid encoding the DNA binding domain of a known transcription factor. In the other construct, a DNA sequence, from a library of sequences, that encodes a potential test agent polypeptide is fused to a gene or nucleic acid that codes for the activation domain of the known transcription factor. If the polypeptide of interest and the test polypeptide interact, the DNA-binding and activation domains are brought into close proximity, which allows transcription of a reporter gene that is operably linked to a transcriptional regulatory site responsive to the transcription factor. Expression of the reporter gene can be detected, and cell colonies containing the functional transcription factor isolated. From these colonies, the nucleic acid molecule encoding the test polypeptide that binds or interacts with the protein involved in intracellular calcium modulation can be isolated.

Another cell-based assay for identification of molecules that interact with a protein, such as a protein involved in modulating intracellular calcium, is the tandem affinity purification (TAP) method (see, e.g., Rigaut et al. (1999) Nature Biotech. 17:1030-1032; Puig et al. (2001) Methods 24:218-229). The method involves a two-step process for the affinity purification of protein complexes from cells, e.g., yeast, when expressed at relatively normal levels under native conditions. The method employs a tag that is fused to the protein of interest (e.g., a protein involved in modulating intracellular calcium) through recombinant expression of the fusion protein in order to facilitate affinity purification using two different selection molecules. The tag can be a fusion of protein A IgG-binding domains (for use in a first affinity purification by an IgG matrix) and a calmodulin-binding peptide (for use in a second affinity purification by calmodulin-coated beads) separated by a TEV protease cleavage site (to eluate protein complexes bound to the IgG matrix). Thus, the protein of interest is expressed in cells as a fusion with the tag and then extracts of the cells are mixed with the IgG matrix, bound complexes are eluted by cleavage with TEV protease, and the eluate is mixed with calmodulin-coated beads in the presence of calcium. The complexes are released from the beads with EGTA. The purified complex can be used for protein identification, functional or structural studies. Analytical methods that can be applied include analytical gel electrophoresis and mass spectrometry. The method can be used to identify a variety of interacting agents, including, for example, proteins, nucleic acids, lipids and peptides.

Assays that generally can be used for identifying test agents that modulate the binding or interaction of a protein of interest (e.g., a protein involved in intracellular calcium modulation) with other molecules are also known in the art. In these assays, a complex of the protein of interest and a binding/interaction partner is typically contacted with a potential test agent and the binding or interaction of the complex is assessed (for example, for an effect of the potential test agent on the binding, such as decreased, increased or other alterations in the interaction) using standard analytical techniques as described herein and known in the art.

Level and Activity Assays

A number of in vitro and cell-based assays are known in the art and can be modified as needed by one of skill in the art to identify agents that alter the level and/or activity of proteins. The assay used can depend on the protein of interest and its activities. Thus, for example, if the protein of interest is an ion transport protein, one method for assessing activity is the analysis of the electrophysiological properties of the channel activity using procedures described herein and known in the art. If the protein of interest has an enzymatic activity, then one method for assessing activity is the analysis of substrate interaction and reaction. For example, if the protein of interest has a protease activity, it typically can be assessed by analysis of substrate cleavage. Potential test agents that modulate an activity of a protein involved in intracellular calcium modulation can be identified by evaluating the effect of such an agent on the particular activity of the protein.

Potential test agents that modulate the levels of a protein involved in intracellular calcium modulation can be identified using a number of techniques known in the art. For example, potential agents that modulate the levels of expression of a protein can be identified in cell-based assays in which the level of the protein (either an endogenous protein or a recombinant or reporter protein) is assessed upon exposure of the cell to a potential test agent. Thus, for example, if the protein of interest is endogenously or recombinantly expressed in a cell using transcription regulatory sequences (e.g., promoters, enhancers) from the gene that encodes the protein, it is possible to identify test agents that modulate expression of the gene by evaluating protein levels of the cell. Alternatively, the transcription regulatory sequences of the gene can be operably linked to DNA encoding a readily measurable reporter protein and the levels of reporter protein measured as an indication of the effect of a potential test agent on expression levels of the protein of interest. Methods of measuring protein levels are well known in the art, including, for example, quantitative electrophoretic analyses and immunoassays, e.g., ELISA and other assays. The levels of the mRNA transcript of a protein of interest can also be determined upon exposure of a cell to a potential test agent to identify agents that modulate expression of the protein. Methods of evaluating mRNA levels are also well known in the art, including, for example, northern blots and RT-PCR.

b. Monitoring The effects of Test Agent on Intracellular Calcium

In this embodiment of the methods of screening for or identifying an agent that modulates intracellular calcium, the effects of a test agent (which is one that binds to, interacts with and/or modulates interactions, activities, levels or any physical, structural or other property of a protein involved in modulating intracellular) on intracellular calcium are monitored. A test agent is identified as an agent that modulates intracellular calcium if it has an effect on intracellular calcium. In particular embodiments, the effect of test agent on store-operated calcium entry, intracellular organelle (e.g., endoplasmic reticulum or other intracellular compartment) calcium storage, uptake or release and/or, resting cytosolic calcium levels is monitored or assessed.

Generally, in monitoring the effect of a test agent on intracellular calcium in any of the screening/identification methods provided herein, including this particular embodiment, some direct or indirect evaluation or measurement of cellular (including cytosolic and intracellular organelle or compartment) calcium and/or movement of ions into, within or out of a cell, organelle, or portions thereof (e.g., a membrane) is conducted. A variety of methods are described herein and/or known in the art for evaluating calcium levels and ion movements or flux. The particular method used and the conditions employed can depend on whether a particular aspect of intracellular calcium is being monitored. For example, as described herein, reagents and conditions are known, and can be used, for specifically evaluating store-operated calcium entry, resting cytosolic calcium levels and calcium levels and uptake by or release from intracellular organelles. The effect of test agent on intracellular calcium can be monitored using, for example, a cell, an intracellular organelle or storage compartment, a membrane (including, e.g., a detached membrane patch or a lipid bilayer) or a cell-free assay system.

In a particular embodiment of these methods, the effect of test agent on intracellular calcium is monitored using a cell. The cell can be one that contains the particular protein involved in intracellular calcium modulation that the test agent binds to, interacts with, and/or modulates the interactions, activities, levels or any physical, structural or other property of. Alternatively, the cell may not contain the particular protein involved in intracellular calcium modulation. In one embodiment the cell used in the method is a cell that exhibits store-operated calcium entry, and the effects of the test agent on store-operated calcium entry are monitored or assessed. 1

4. Methods Based on Testing the Effect of Any Test Agent on Intracellular Calcium

In another embodiment of the methods for screening for or identifying agents that modulate intracellular calcium provided herein, the effect of any test agent on intracellular calcium (and, in particular embodiments, the effect on store-operated calcium entry, intracellular organelle calcium storage, uptake or release and/or, resting cytosolic calcium levels) is monitored, assessed or evaluated. In this embodiment of the methods, the test agent can be any agent, and is not necessarily one that (or has been identified as one that) binds to, interacts with and/or modulates interactions, activities, levels or any physical, structural or other property of a protein involved in modulating intracellular calcium. Generally, in this embodiment, the method can be performed by contacting any test agent with (1) one or more proteins involved in modulating intracellular calcium, e.g., an ion transport protein, and/or (2) a cell, or portion thereof, e.g., a membrane, containing one or more proteins involved in modulating intracellular calcium, such as an ion transport protein, and/or nucleic acid (e.g., a gene or coding sequence such as cDNA or RNA), or portion(s) thereof, encoding such proteins. The one or more proteins involved in modulating intracellular calcium that are used in these methods can be proteins as provided herein above. In particular embodiments, the one or more proteins is (are) one that is involved in, participates in, and/or provides for store-operated calcium entry, maintenance of resting cytosolic calcium levels and/or calcium storage in, or movement of cations into or out of, an intracellular organelle or compartment (e.g., endoplasmic reticulum). In one embodiment of the methods, the effect of test agent on store-operated calcium entry is monitored.

The monitoring, evaluation or assessment of intracellular calcium in these embodiments of the methods can be conducted in a variety of ways which can be used for all embodiments of the screening/identification methods as described herein. The monitoring typically involves some direct or indirect evaluation or measurement of cellular (including cytosolic and intracellular organelle or compartment) calcium and/or movement of ions into, within or out of a cell, organelle, or portions thereof (e.g., a membrane) is conducted. A number of methods are described herein and/or known in the art for evaluating calcium levels and ion movements or flux.

5. Cells

Any cell that can be evaluated for intracellular calcium may be used in the methods provided herein. In a particular embodiment, the cell is one in which store-operated calcium entry occurs or that can be manipulated such that store-operated calcium entry occurs in the cell. In particular embodiments, the cell contains one or more proteins involved in modulating intracellular calcium (and, in particular, is involved in, participates in and/or provides for store-operated calcium entry, intracellular organelle calcium storage, uptake or release and/or, maintenance of resting cytosolic calcium levels), such as those provided herein. The cell may endogenously express the protein(s) or recombinantly express the protein(s) through introduction of heterologous nucleic acid encoding the protein(s) into the cell using methods known in the art and described herein. In particular embodiments, the cell is a recombinant cell that expresses the protein(s) as heterologous protein(s). Such cells may overexpress the heterologous protein(s). For example, a recombinant cell may be one that endogenously expresses the protein(s) and in also has been transfected with additional copies of nucleic acid encoding the protein(s). In a particular example, the host cell used in the generating the recombinant cell may be one that endogenously expresses little to no store-operated calcium entry activity (e.g., CHO-K1 cells which do not exhibit a current with biophysical properties characteristic of a store-operated calcium entry current), or a host cell in which endogenous store-operated calcium entry activity has been eliminated (e.g., through gene knock-out or silencing, such as RNA interference, methods or by inhibition with an agent that does not inhibit store-operated calcium entry activity of the heterologous protein(s)).

Cells or less differentiated precursor cells having an endogenous protein involved in modulating intracellular calcium can be used. Cells or less differentiated precursor cells may be recombinant cells stably or transiently transfected with intracellular calcium-modulating protein(s) in vitro or in an organism. In vitro transfection is followed by cell expansion through culturing prior to use.

Cells for use in the methods may be of any species. In one embodiment, the cells can be eukaryotic cells. In a particular embodiment, the cells can be yeast, insect (e.g., Drosophila or Anopheles), or mammalian cells. Mammalian cells include, but are not limited to, rodent (e.g., mouse, rat and hamster), primate, monkey, dog, bovine, rabbit and human cells. Particular cells include Drosophila Schneider 2 or S2 cells, human embryonic kidney (HEK) cells, neuronal, brain or nervous system cells, rat basophilic leukemia (RBL) cells, and immune system cells, e.g., lymphocytes such as T lymphocytes, including Jurkat cells.

In particular embodiments, neuronal, nervous system (e.g., CNS) or tissue-derived or brain cells that contain (endogenously and/or recombinantly) one or more proteins involved in modulating intracellular calcium as described above may be used in the methods of identifying agents that modulate intracellular calcium. Cells from a known cell line can be used, such as neuroblastoma SH-SY5Y cells, pheochromocytoma PC 2 cells, neuroblastoma SK-N-BE(2)C cells, human SK-N-MC neuroblastoma cells, SMS-KCNR cells, human LAN-5 neuroblastoma cells, human GI-CA-N neuroblatoma cells, human GOTO neuroblastoma cells, mouse Neuro 2a (N2A) neuroblastoma cells and/or human IMR 32 neuroblastoma cells. Primary cells, e.g., dorsal root ganglion and other primary neuronal or CNS-derived cells, can also be used in the methods. Other cell lines include HEK 293, CHO (including PZ3 CHO and CHO-K1), LTK-, N2A, H6, and HGB. The generation, maintenance and use of such cell lines is well known. The host cell for generation of a recombinant cell may be a less differentiated precursor cell, and preferably is one that is readily stably transfected.

The choice of a cell for use in practicing any of the methods for screening for or identifying an agent involved in modulating intracellular calcium can involve several considerations, including, for example, a particular protein that is being used in the method and a particular aspect or activity of intracellular calcium modulation that is being monitored in the method. For example, different cells (e.g., different cell types or cells of the same type from different species of organisms) may have different sets of molecules, including proteins and ion transport proteins, that participate in various aspects (e.g., store-operated calcium entry, receptor-mediated calcium movement, second messenger-operated calcium movement, calcium uptake, storage and/or release from intracellular compartments) of intracellular calcium modulation. Accordingly, a cell used in the methods can be selected to be one that provides (endogenously and/or recombinantly) a cellular environment conducive for functioning of any particular protein(s) (such as those provided herein and described above) being used in the method or supportive of any particular activity in intracellular calcium modulation that is being specifically monitored in the methods. Additionally, some cells may be more amenable to various manipulations that may be a part of some embodiments of the methods, e.g., recombinant expression of proteins, growth in culture, electrophysiological analysis, etc. Cells that are particularly suitable for specific embodiments of the methods can be determined empirically by those of skill in the art.

6. Monitoring of Effects on Intracellular Calcium

In monitoring the effect of a test agent on intracellular calcium in any of the screening/identification methods provided herein, a direct or indirect evaluation or measurement of cellular (including cytosolic and intracellular organelle or compartment) calcium and/or movement of ions into, within or out of a cell, organelle, or portions thereof (e.g., a membrane) can be conducted. A variety of methods are described herein and/or known in the art for evaluating calcium levels and ion movements or flux. The particular method used and the conditions employed can depend on whether a particular aspect of intracellular calcium is being monitored. For example, as described herein, reagents and conditions are known, and can be used, for specifically evaluating store-operated calcium entry, resting cytosolic calcium levels and calcium levels and uptake by or release from intracellular organelles. The effect of test agent on intracellular calcium can be monitored using, for example, a cell, an intracellular organelle or storage compartment, a membrane (including, e.g., a detached membrane patch or a lipid bilayer) or a cell-free assay system.

Generally, monitoring the effect of a test agent on intracellular calcium involves contacting a test agent with or exposing a test agent to (1) a protein (and/or nucleic acid, or portion(s) thereof, encoding a protein) involved in modulating intracellular calcium (in particular, a protein provided herein) and/or (2) a cell, or portion(s) thereof (e.g., a membrane or intracellular structure or organelle) that may or may not contain a protein (and/or nucleic acid, or portion(s) thereof, encoding a protein) involved in modulating intracellular calcium. A cell can be one that exhibits one or more aspects of intracellular modulation, such as, for example, store-operated calcium entry. Before, during and/or after the contacting of test agent, a direct or indirect assessment of intracellular calcium can be made. An indirect assessment can be, for example, evaluation or measurement of current through an ion transport protein (e.g., a store-operated calcium channel), or transcription of a reporter protein operably linked to a calcium-sensitive promoter. A direct assessment can be, for example, evaluation or measurement of intracellular (including cytosolic and intracellular organelle) calcium.

The assessment of intracellular calcium is made in such a way as to be able to determine an effect of an agent on intracellular calcium. Typically, this involves comparison of intracellular calcium in the presence of a test agent with a control for intracellular calcium. For example, one control is a comparison of intracellular calcium in the presence and absence of the test agent or in the presence of varying amounts of a test agent. Thus, one method for monitoring an effect on intracellular calcium involves comparing intracellular calcium before and after contacting a test agent with a test cell containing a protein that modulates intracellular calcium, or comparing intracellular calcium in a test cell that has been contacted with test agent and in a test cell that has not been contacted with test agent (i.e., a control cell). Generally, the control cell is substantially identical to, if not the same as, the control cell, except it is the cell in the absence of test agent. A difference in intracellular calcium of a test cell in the presence and absence of test agent indicates that the agent is one that modulates intracellular calcium.

Another method for monitoring an effect on intracellular calcium involves comparing intracellular calcium of a test cell and a control cell that is substantially similar to the test cell (e.g., a cell containing a protein (and/or nucleic acid encoding a protein) involved in intracellular calcium, such as the proteins provided herein) but that does not contain the particular protein involved in modulating intracellular calcium. Thus, for example, if the test cell containing the protein involved in intracellular calcium modulation is a recombinant cell generated by transfer of nucleic acid encoding the protein into a host cell, then one possible control cell is a host cell that has not been transfected with nucleic acid encoding the protein. Such a cell would be substantially similar to the test cell but would differ from the test cell essentially only by the absence of the introduced nucleic acid encoding the protein. Thus, a control cell may contain, e.g., endogenously, the particular protein involved in modulating intracellular calcium, in which case the test cell would contain higher levels of (or overexpress) the particular protein. This type of control comparison is particularly of use when it is desired to identify an agent that specifically modulates intracellular calcium via an effect on, or modulation of, a particular protein (and/or nucleic acid, or portion(s) thereof, encoding a particular protein). Thus, for example, if there is no detectable or substantial difference in intracellular calcium in the test and control cells in the presence of the agent, it is not likely that the agent has an effect on intracellular calcium that is specifically mediated via the particular protein (or nucleic acid encoding the protein). If there is a detectable or substantial difference in intracellular calcium in the test and control cells in the presence of the test agent, then the test agent may be a candidate agent that specifically modulates intracellular calcium via an effect on or modulation of the particular protein. A candidate agent can be subjected to further control assays to compare intracellular calcium in test cells in the presence and absence of test agent or to compare intracellular calcium in control cells in the presence and absence of test agent, which can aid in determination of whether a candidate agent is an agent that modulates intracellular calcium.

An assessment of intracellular calcium conducted to monitor the effect of test compound on intracellular calcium can be made under a variety of conditions. Conditions can be selected to evalulate the effect of test compound on a specific aspect of intracellular calcium. For example, as described herein, reagents and conditions are known, and can be used, for specifically evaluating store-operated calcium entry, resting cytosolic calcium levels and calcium levels of and calcium uptake by or release from intracellular organelles. For example, as described herein, calcium levels and/or calcium release from the endoplasmic reticulum can directly be assessed using mag-fura 2, endoplasmic reticulum-targeted aequorin or cameleons. One method for indirect assessment of calcium levels or release is monitoring intracellular calcium levels (for example using fluorescence-based methods) after exposing a cell to an agent that effects calcium release (actively, e.g., IP₃, or passively, e.g., thapsigargin) from the organelle in the absence of extracellular calcium.

Resting cytosolic calcium levels, intracellular organelle calcium levels and cation movement may be assessed using any of the methods described herein or known in the art (see, e.g., descriptions herein of calcium-sensitive indicator-based measurements, such as fluo-3, mag-fura 2 and ER-targeted aequorin, labelled calcium (such as ⁴⁵Ca²⁺)-based measurements, and electrophysiological measurements). Particular aspects of ion flux that may be assessed include, but are not limited to, a reduction (including elimination) or increase in the amount of ion flux, altered biophysical properties of the ion current, and altered sensitivities of the flux to activators or inhibitors of calcium flux processes, such as, for example, store-operated calcium entry. Reagents and conditions for use in specifically evaluating receptor-mediated calcium movement and second messenger-operated calcium movement are also available.

In particular embodiments of the methods for screening for or identifying agents that modulate intracellular calcium, the methods are conducted under conditions that permit store-operated calcium entry to occur (see, e.g., the EXAMPLES). Such conditions are described herein and are known in the art. Test agents can be contacted with a protein and/or nucleic acid encoding a protein (such as the proteins and nucleic acids provided herein) involved in modulating intracellular calcium and/or a cell (or portion thereof) containing such a protein (or nucleic acid) under these appropriate conditions. For example, in conducting one method for screening for an agent that modulates intracellular calcium under conditions selected for evaluating store-operated calcium entry, intracellular calcium levels of test cells are monitored over time using a fluorescent calcium indicator (e.g., FLUO-4). Store-operated calcium entry into the cells is detected as an increase in fluorescence (i.e., increase in intracellular calcium levels) in response to conditions under which store-operated calcium entry occurs. The conditions include addition of a store-depletion agent, e.g., thapsigargin (which inhibits the ER calcium pump and discharge calcium stores) to the media of cell that has been incubated in Ca²⁺-free buffer, incubation with thapsigargin for about 5-15 minutes, addition of test compound (or vehicle control) to the media and incubation of the cell with test agent for about 5-15 minutes, followed by addition of external calcium to the media to a final concentration of about 1.8 mM. By adding thapsigargin to the cell in the absence of external calcium, it is possible to delineate the transient increase in intracellular calcium levels due to calcium release from calcium stores and the more sustained increase in intracellular calcium levels due to calcium influx into the cell from the external medium (i.e., store-operated calcium entry through the plasma membrane that is detected when calcium is added to the medium). Because the fluorescence-based assay allows for essentially continous monitoring of intracellular calcium levels during the entire period from prior to addition of thapsigargin until well after addition of calcium to the medium, not only can “peak” or maximal calcium levels (as well as total calcium entry) resulting from store-operated calcium entry be assessed in the presence and absence of test agent, a number of other parameters of the calcium entry process may also be evaluated, as described herein. For example, the kinetics of store-operated calcium entry can be assessed by evaluation of the time required to reach peak intracellular calcium levels, the up slope and rate constant associated with the increase in calcium levels, and the decay slope and rate constant associated with the decrease in calcium levels as store-operated calcium entry discontinues. Any of these parameters can be evaluated and compared in the presence and absence of test agent to determine whether the agent has an effect on store-operated calcium entry, and thus on intracellular calcium. In other embodiments, store-operated calcium entry can be evaluated by, for example, assessing a current across a membrane or into a cell that is characteristic of a store-operated calcium entry current (e.g., responsiveness to reduction in calcium levels of intracellular stores) or assessing transcription of reporter construct that includes a calcium-sensitive promoter element. In particular embodiments, a test agent is identified as one that produces at least a 50% difference in any aspect or parameter of store-operated calcium entry relative to control (e.g., absence of compound, i.e., vehicle only).

Generally, a test agent is identified as an agent, or candidate agent, that modulates intracellular intracellular calcium if there is a detectable effect of the agent on intracellular calcium levels and/or ion movement or flux, such as a detectable difference in levels or flux in the presence of the test agent. In particular embodiments, the effect or differences can be substantial or statistically significant.

G. Systems

Also provided herein are systems for use in identifying an agent that modulates intracellular or cytoplasmic calcium. Such systems include a cell, or portion(s) thereof, containing one or more proteins, e.g., ion transport proteins (and/or nucleic acids or portions thereof encoding proteins), involved in modulating intracellular calcium (and, in particular embodiments, that is involved in, participates in, and/or provides for store-operated calcium entry, intracellular organelle calcium storage, uptake or release, and/or maintenance of resting cytosolic calcium levels) that has an amino acid sequence that is at least about 20%, or at least about 25%, or at least about 30%, or at least about 35%, or at least about 40%, or at least about 45%, at least about 46%, or at least about 47%, or at least about 48%, or at least about 49%, or at least about 50%, or at least about 52%, or at least about 55%, or at least about 60%, or at least about 65%, or at least about 70%, or at least about 75%, or at least about 80%, or at least about 85%, or at least about 90% or at least about 95% or more homologous to the protein encoded by the coding sequence of at least Drosophila gene CG5842 (Genbank Accession No. AAF49752; gi7294406 or SEQ ID NO: 4) or gene CG4536 (Genbank Accession No. AAF46203; gi7290757 or SEQ ID NO: 2). In particular embodiments, the protein is at least about 35%, or at least about 43% or at least about 44%, or at least about 45%, or at least about 46%, or at least about 47%, or at least about 48% or at least about 50% homologous to one or both of the proteins encoded by Drosophila genes CG4536 and CG5842. Such exemplary proteins may be homologous to the specified Drosophila proteins over at least about 25%, or at least about 27%, or at least about 30%, or at least about 35%, or at least about 40%, or at least about 43%, or at least about 45%, or at least about 50%, or at least about 51%, or at least about 52%, or at least about 53%, or at least about 54% or at least about 55%, or at least about 60%, or at least about 65%, or at least about 70%, or at least about 75%, or at least about 80%, or at least about 85%, or at least about 90%, or at least about 95% or more of the protein encoded by the coding sequence of at least Drosophila gene CG5842 (Genbank Accession No. AAF49752; gi7294406 or SEQ ID NO: 4) and/or gene CG4536 (Genbank Accession No. AAF46203; gi7290757 or SEQ ID NO: 2). In a particular embodiment, the protein is homolgous to the Drosophila protein over at least about 25%, or at least about 35%, or at least about 40%, or at least about 43%, or at least about 45%, or at least about 50%, or at least about 51%, or at least about 52% or more of the protein encoded by the coding sequence of at least Drosophila gene CG5842 (Genbank Accession No. AAF49752; gi7294406 or SEQ ID NO: 4) or gene CG4536 (Genbank Accession No. AAF46203; gi7290757 or SEQ ID NO: 2). In further particular embodiments, the protein(s) is/are any of those specified above with respect to the methods for screening for or identifying an agent that modulates intracellular calcium.

In particular embodiments of the systems, the protein(s) involved in modulating intracellular calcium are contained in cells. The cells can be isolated cells or cell cultures that endogenously express such protein(s) or recombinantly express such proteins as described above with respect to the methods for identifying agents. Systems in which the cells recombinantly express the proteins can be such that the cells are isolated cells or cell cultures or are contained within an animal,-in particular, a non-human animal, e.g., a non-human mammal.

The proteins (and/or nucleic acids encoding proteins) or cells (or portions thereof) of the sytem can be contained in a medium that contains an agent that provides for passive or active intracellular calcium store reduction or depletion (e.g., thapsigargin and other agents described herein or known in the art) and/or that contains a molecule or molecules that facilitate monitoring or measurement of intracellular calcium and/or calcium movement. Such molecules include fluorescent (or otherwise labeled) calcium indicators, trivalent cations, divalent cations other than calcium and calcium-buffering agents, e.g., calcium chelators.

H. Methods of Identifying Molecules Involved in Modulating Intracellular Calciums

Proteins (and/or nucleic acids encoding proteins) involved in modulating intracellular calcium, as described herein, further provide the basis for additional methods of identifying molecules involved in modulating intracellular calcium, as well as for methods of elucidating pathways, and elements thereof, of intracellular calcium modulation. Once a protein has been identified as one involved in modulating intracellular calcium, it can be used to identify molecules, in particular cellular components, that interact with it and that potentially function in the modulation of intracellular calcium. Additionally, the identification of molecules that interact with proteins involved in intracellular calcium modulation facilitates the dissection and elucidation of pathways and mechanisms of cellular calcium regulation and signaling. The elucidation of such pathways provides additional targets that can be modulated in methods of modulating intracellular calcium and for use in methods of identifying agents for modulating intracellular calcium. Because the dissection of such pathways also elucidates components of the pathway and interactions between the components, it further makes possible the refinement of methods of modulating intracellular calcium by the balanced targeting of modulation of one or more components and/or interactions of the pathway.

Particular proteins (and/or nucleic acids encoding proteins) involved in modulating intracellular calcium (and, in particular, proteins that are involved in, participate in, and/or provide for store-operated calcium entry, intracellular organelle calcium storage, uptake or release, and/or maintenance of resting cytosolic calcium levels) are provided herein. These proteins can thus be used in methods to identify molecules, in particular cellular components, that interact with the proteins and that may be involved in modulating intracellular calcium.

Provided herein are methods of identifying a candidate molecule involved in modulating intracellular calcium (and, in particular, that are involved in, participate in, and/or provide for store-operated calcium entry, intracellular organelle calcium storage, uptake or release, and/or maintenance of resting cytosolic calcium levels). The methods include a step of identifying a molecule, such as, for example, a cellular component, that interacts with a protein involved in intracellular calcium modulation.

1. Proteins Involved in Intracellular Calcium Modulation

In one embodiment of the methods of identifying a candidate molecule involved in modulating intracellular calcium, the method includes a step of identifying a molecule, such as, for example, a cellular component, that interacts with the protein (or portion thereof) encoded by Drosophila gene CG4536 and/or CG5842, or a portion thereof. The candidate molecule can then be further tested for intracellular calcium-modulating properties by evaluating the effect of modulating the molecule and/or its interaction with the protein encoded by Drosophila gene CG4536 and/or CG5842 on intracellular calcium.

In another embodiment of the methods for identifying a candidate molecule involved in modulating intracellular calcium, the method includes a step of identifying a molecule, such as, for example, a cellular component, that interacts with a protein (or portion thereof) that is homologous to the protein encoded by Drosophila gene CG4536 and/or CG5842. In particular embodiments of these methods, the protein that is homologous to the protein encoded by Drosophila gene CG4536 and/or CG5842 has an amino acid sequence that is at least about 20%, or at least about 25%, or at least about 30%, or at least about 35%, or at least about 40%, or at least about 45%, at least about 46%, or at least about 47%, or at least about 48%, or at least about 49%, or at least about 50%, or at least about 52%, or at least about 55%, or at least about 60%, or at least about 65%, or at least about 70%, or at least about 75%, or at least about 80%, or at least about 85%, or at least about 90% or at least about 95% or more homologous to the protein encoded by the coding sequence of at least Drosophila gene CG5842 (Genbank Accession No. AAF49752; gi7294406 or SEQ ID NO: 4) or gene CG4536 (Genbank Accession No. AAF46203; gi7290757 or SEQ ID NO: 2). In particular embodiments, the protein is at least about 35%, or at least about 43% or at least about 44%, or at least about 45%, or at least about 46%, or at least about 47%, or at least about 48% or at least about 50% homologous to one or both of the proteins encoded by Drosophila genes CG4536 and CG5842. Such exemplary proteins may be homologous to the specified Drosophila proteins over at least about 25%, or at least about 27%, or at least about 30%, or at least about 35%, or at least about 40%, or at least about 43%, or at least about 45%, or at least about 50%, or at least about 51%, or at least about 52%, or at least about 53%, or at least about 54% or at least about 55%, or at least about 60%, or at least about 65%, or at least about 70%, or at least about 75%, or at least about 80%, or at least about 85%, or at least about 90%, or at least about 95% or more of the protein encoded by the coding sequence of at least Drosophila gene CG5842 (Genbank Accession No. AAF49752; gi7294406 or SEQ ID NO: 4) and/or gene CG4536 (Genbank Accession No. AAF46203; gi7290757 or SEQ ID NO: 2). In a particular embodiment, the protein is homolgous to the Drosophila protein over at least about 25%, or at least about 35%, or at least about 40%, or at least about 43%, or at least about 45%, or at least about 50%, or at least about 51%, or at least about 52% or more of the protein encoded by the coding sequence of at least Drosophila gene CG5842 (Genbank Accession No. AAF49752; gi7294406 or SEQ ID NO: 4) or gene CG4536 (Genbank Accession No. AAF46203; gi7290757 or SEQ ID NO: 2). In further particular embodiments, the protein(s) is/are any of those specified above with respect to the methods for screening for or identifying an agent that modulates intracellular calcium.

2. Assaying for an Interaction between a Molecule and a Protein Involved in Modulating Intracellular Calcium

The methods provided herein for identifying a candidate molecule involved in modulating intracellular calcium, involve identifying an interaction between a molecule, such as, for example, a cellular component, and a protein (or portion thereof) involved in intracellular calcium modulation (and, in particular, that are involved in, participate in, and/or provide for store-operated calcium entry, intracellular organelle calcium storage, uptake or release, and/or maintenance of resting cytosolic calcium levels). The interaction can be any direct or indirect physical, biochemical, chemical or other interaction between a molecule and a protein involved in intracellular calcium modulation such as, for example, those described herein. There are a number of methods described herein and/or known in the art for identifying an interaction between a protein and other molecules, e.g., cellular components. Several such methods are described herein with respect to the methods for screening for or identifying agents that modulate intracellular calcium.

For example, one type of interaction that can occur between a candidate molecule involved in modulating intracellular calcium and a protein (or portion thereof) involved in intracellular calcium modulation is a binding interaction. A number of in vitro and cell-based binding assays are known in the art and can be modified as needed by one of skill in the art to identify molecules that bind to or interact with a protein involved in modulating intracellular calcium. In an one method, the protein involved in intracellular calcium modulation is contacted with cellular components (within the context of a cell or in isolation), cell medium, a cell and/or a cell extract and assayed for binding of a molecule to the protein involved in modulating intracellular calcium. Binding can be evaluated and detected using any of several methods known in the art for detecting binding of molecules to proteins, including, but not limited to, affinity chromatography (including Biacore and Ciphergen technologies), immunoprecipitation, ELISA assays, far-western blotting and other methods. Immunoassays to detect binding of molecules to a protein involved in intracellular calcium modulation can utilize antibodies (e.g., monoclonal and polyclonal) prepared against the protein or portions thereof. Methods of generating and testing antibodies against proteins are well known in the art. Interactions between a protein involved in intracellular calcium modulation and a candidate molecule involved in modulating intracellular calcium can also be identified using assays such as co-purification assays (e.g., GST pull-down assays, co-immunoprecipitation assay, chromatographic assays), phage display, ribozyme display, and protein arrays. Detection of in vitro binding or interaction between a candidate molecule and a protein involved in modulating intracellular calcium can involve a variety of approaches, such as, for example, nuclear magnetic resonance (NMR), mass spectroscopy, fluorescence spectroscopy, scintillation proximity assays (SPQ), surface plasmon resonance assays (available commercially from BIACORE; http://www.biacore.se/proteomics/), and others. Cell-based binding assays include, for example, yeast two-hybrid assays (see, e.g., U.S. Pat. Nos. 5,283,173, 5,468,614 and 5,667,973); bacterial two-hybrid assays (Juong (2001) J. Cell Biochem. Suppl. 37:53-57), and others. Such assays are particularly suitable for identifying polypeptides that interact with a protein involved in modulating intracellular calcium. Another cell-based assay for identification of molecules that interact with a protein, such as a protein involved in modulating intracellular calcium, is the tandem affinity purification (TAP) method (see, e.g., Rigaut et al. (1999) Nature Biotech. 17:1030-1032; Puig et al. (2001) Methods 24:218-229).

A molecule that has bound to the protein (or portion thereof) involved in intracellular calcium modulation can be characterized and identified using methods that are also well known in the art, including, for example, HPLC, FPLC, amino acid sequencing if the molecule is a protein, and cloning of nucleic acid encoding a molecule that is a protein.

Once a molecule is found to interact with a protein involved in modulating intracellular calcium, it can be further evaluated to assess its intracellular calcium-modulating properties. For example, a cell comprising the interacting molecule and the protein involved in modulating intracellular calcium can be used for such evaluations. The interaction between the molecule and the protein involved in intracellular calcium modulation can be disrupted or altered in the cell and then intracellular calcium can be monitored to determine if intracellular calcium is altered by the alteration in the interaction. Intracellular calcium can be assessed using any methods known in the art or described herein. In particular methods, store-operated calcium entry, resting cytosolic calcium levels and/or calcium levels in or ion movement into or out of an intracellular organelle is assessed. In another process for assessing the intracellular calcium-modulating properties of an interacting molecule, the amount of the molecule in such a cell (or any cell containing at least the interacting molecule) can also be altered, for example, increased, decreased or the molecule can be eliminated in the cell, and the effect of such alteration on intracellular calcium can be assessed. One method for altering the amount of the molecule in the cell, if the molecule is a protein produced by the cell, is to alter the expression of the protein in the cell. This can be accomplished using a variety of methods, including methods described herein, such as for example, RNA interference, antisense RNA methods, gene knock-out procedures and gene insertion/over-expression processes.

I. Methods of Modulating Intracellular Calcium

Provided herein are methods for modulating intracellular calcium. Modulation of intracellular calcium can be any alteration or adjustment in intracellular calcium including but not limited to alteration of calcium concentration or level in the cytoplasm and/or intracellular calcium storage organelles, e.g., endoplasmic reticulum, alteration in the movement of calcium into, out of and within a cell, alteration in the location of calcium within a cell, and alteration of the kinetics, or other properties, of calcium fluxes into, out of and within cells. In particular embodiments, intracellular calcium modulation can involve an alteration in basal or resting cytosolic calcium levels, store-operated calcium entry and/or calcium levels in or ion movement into or out of an intracellular organelle. In other embodiments, modulation of intracellular calcium can involve an alteration in receptor-mediated ion (e.g., calcium) movement, second messenger-operated ion (e.g., calcium) movement, calcium influx into or efflux out of a cell, and/or ion (e.g., calcium) uptake into or release from intracellular compartments, including, for example, endosomes and lysosomes.

Methods of modulating intracellular calcium provided herein include a step of modulating the level of, expression of, activity of or molecular interactions of a protein in a cell that has altered intracellular calcium, and, in particular, altered store-operated calcium entry, altered resting cytosolic calcium levels and/or altered calcium levels in or ion movement into or out of an intracellular organelle. The protein that is modulated can be, for example, an ion transport protein, calcium-binding protein or a protein that regulates an ion transport protein. In a particular embodiment, the protein is homologous to a protein encoded by a Drosophila gene that, when altered in its expression in a Drosophila cell, results in altered store-operated calcium entry into the cell. An alteration of store-operated ion flux into a cell can be a complete or nearly complete elimination of the activity, a reduction of the activity, an alteration in properties or characteristics (e.g., current properties or sensitivities) of the activity or an increase in the activity relative to the activity in a control cell (e.g., a cell such as a Drosophila cell) that has not been altered in its store-operated ion flux activity. Similarly, an alteration in gene expression may be complete or nearly complete elimination of the expression of a gene, a reduction in the expression of a gene, an increase in the expression of a gene, or an alteration in the protein encoded by the gene (such as truncation or other alteration that effectively renders the protein nonfunctional or provides for aberrant functioning of the protein) relative to the expression of the gene in a cell that has not been altered in its expression of the gene.

In another embodiment, the protein is homologous to a protein encoded by a Drosophila gene that, when altered in its expression in a Drosophila cell, results in altered intracellular calcium in the cell. An alteration in intracellular calcium can be any alteration in calcium level, movement, location, or other calcium alteration, in a cell. Thus, in one example, an alteration in intracellular calcium can be an alteration in the calcium level within an intracellular organelle or calcium storage compartment or an alteration in basal or resting cytosolic calcium levels. An alteration of intracellular calcium can be any change in intracellular calcium compared to intracellular calcium in a control cell (e.g., a Drosophila cell that does not have altered expression of the gene). Thus, an alteration of intracellular calcium can be, for example, an increase or decrease in basal or resting cytosolic calcium levels compared to control levels (e.g., levels in a Drosophila cell that does not have altered expression of the gene). Assessment of intracellular calcium can be conducted in a number of ways, such as by methods described herein or known in the art. For example, assessment of basal or resting calcium levels can be conducted as described in the EXAMPLES and by using any methods known in the art. An alteration in basal or resting cytosolic calcium can also be effected in a number of ways, including, but not limited to, alterations in calcium flux across the plasma membrane or membranes of intracellular organelles such as the endoplasmic reticulum.

In particular methods, the protein in the cell that is modulated is one that is homologous to the protein encoded by Drosophila gene CG4536 and/or CG5842 and that is involved in modulating intracellular calcium (and, in particular, that is involved in, participates in and/or provides for store-operated calcium entry, maintenance of resting cytosolic calcium levels and/or calcium storage in or uptake or relase of calcium from an intracellular organelle). In particular embodiments of these methods, the protein that is homologous to the protein encoded by Drosophila gene CG4536 and/or CG5842 has an amino acid sequence that is at least about 20%, or at least about 25%, or at least about 30%, or at least about 35%, or at least about 40%, or at least about 45%, at least about 46%, or at least about 47%, or at least about 48%, or at least about 49%, or at least about 50%, or at least about 52%, or at least about 55%, or at least about 60%, or at least about 65%, or at least about 70%, or at least about 75%, or at least about 80%, or at least about 85%, or at least about 90% or at least about 95% or more homologous to the protein encoded by the coding sequence of at least Drosophila gene CG5842 (Genbank Accession No. AAF49752; gi7294406 or SEQ ID NO: 4) or gene CG4536 (Genbank Accession No. AAF46203; gi7290757 or SEQ ID NO: 2). In particular embodiments, the protein is at least about 35%, or at least about 43% or at least about 44%, or at least about 45%, or at least about 46%, or at least about 47%, or at least about 48% or at least about 50% homologous to one or both of the proteins encoded by Drosophila genes CG4536 and CG5842. Such exemplary proteins may be homologous to the specified Drosophila proteins over at least about 25%, or at least about 27%, or at least about 30%, or at least about 35%, or at least about 40%, or at least about 43%, or at least about 45%, or at least about 50%, or at least about 51%, or at least about 52%, or at least about 53%, or at least about 54% or at least about 55%, or at least about 60%, or at least about 65%, or at least about 70%, or at least about 75%, or at least about 80%, or at least about 85%, or at least about 90%, or at least about 95% or more of the protein encoded by the coding sequence of at least Drosophila gene CG5842 (Genbank Accession No. AAF49752; gi7294406 or SEQ ID NO: 4) and/or gene CG4536 (Genbank Accession No. AAF46203; gi7290757 or SEQ ID NO: 2). In a particular embodiment, the protein is homolgous to the Drosophila protein over at least about 25%, or at least about 35%, or at least about 40%, or at least about 43%, or at least about 45%, or at least about 50%, or at least about 51%, or at least about 52% or more of the protein encoded by the coding sequence of at least Drosophila gene CG5842 (Genbank Accession No. AAF49752; gi7294406 or SEQ ID NO: 4) or gene CG4536 (Genbank Accession No. AAF46203; gi7290757 or SEQ ID NO: 2). In further particular embodiments, the protein(s) is/are any of those specified above with respect to the methods for screening for or identifying an agent that modulates intracellular calcium, including, for example, a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp12), a transient receptor potential protein V3 (TRPV3), a stretch-inhibitable nonselective channel (SIC) protein.

Methods of modulating intracellular calcium include a step of modulating the level of, expression of, activity of, functioning and/or molecular interactions of a protein involved in modulating intracellular calcium. For example, the methods may include a step of contacting a cell with an agent that modulates the level, functioning and/or activity of one or more proteins involved in modulating intracellular calcium.

Agents that can modulate the activity of the protein include, but are not limited to, substances that increase, decrease or otherwise alter the activity and/or functioning of the protein. Activities of calcium-modulating proteins include, but are not limited to, calcium transport, calcium binding, and regulation of calcium-modulating proteins. Thus, agents that modulate these activities, or any other activity involved in intracellular calcium modulation, can be used in the methods provided herein. For example, antibodies or other proteins that specifically bind to the protein and modulate such activities can be agents used in the methods. An antibody or other protein may, for instance, bind to a site of a regulatory protein and reduce or eliminate its binding to the protein it regulates, thereby reducing its regulatory activity. Ions other than calcium or molecules, such as small organic molecules, can also be agents that may, for example, reduce or increase calcium transport by an ion transport protein and thus can be agents used in the methods.

J. Diseases

Because of the important role that calcium regulation plays in many cellular processes and, more particularly, the influence that store-operated calcium influx has on a number of cellular functions including cellular activation, gene expression, and apoptotic cell death, store-operated calcium entry or influx (SOCI) is thought to have a role in the many disease and disorders involving such cellular activities. These diseases and disorders include neurological, inflammatory, liver and kidney diseases and disorders, malignancies, aging, sensitivity to pain and sensitivity to touch. Thus, the proteins described herein and their derivatives are useful for studying the processes related to such diseases and disorders and for discovering drugs for treatment. The methods provided for identifying agents that modulate intracellular calcium have uses in discovering agents that have use in studying store-operated calcium entry processes (such as agents that may be specific activators or inhibitors) and also may be used in therapies for such diseases and disorders.

1. Neurological Diseases and Disorders

Neurological diseases and disorders include but are not limited to Alzheimer's disease (AD), Parkinson's disease, Huntington's disease, amyotrophic lateral sclerosis (ALS), and other brain disorders caused by trauma or other insults including aging.

There is ample evidence that mechanisms associated with calcium signaling may be altered in many neurodegenerative diseases and disorders resulting from brain injury. For example, fibroblasts or T-lymphocytes from patients with AD have consistently displayed and increase in Ca2+ release from intracellular stores compared to controls (Ito et al. (1994) PNAS, 91:534-538; Gibson et al., (1996) BBA, 1316:71-77; Etchenberrigaray et al., (1998) Neurobiology of Disease, 5:37-45). Consistent with these observations, mutations in presenilin genes (PS1 or PS2) associated with familial AD (FAD) have been shown to increase InsP3-mediated Ca2+ release from internal stores (Guo et al., (1996) Neuro Report, 8:379-383; Leissring et al., (1999) J. Neurochemistry, 72:1061-1068; Leissring et al., (1999) JBC, 274(46):32535-32538; Leissring et al., (2000) JBC, 149(4):793-797; Leissring et al., (2000) PNAS, 97(15):8590-8593). Furthermore, Yoo et al. ((2000) Neuron, 27(3):561-572) have shown that mutations in PS1 or PS2 that increase the amyloidogenic amyloid β peptide (Aβ42) generation in AD are associated with a decrease in CCE, and that direct pharmacological blockage of CCE increases Aβ42 levels in a concentration-dependent manner.

Experimental traumatic brain injury has been shown to initiate massive disturbances in Ca2+ concentrations in the brain that may contribute to further neuronal damage. Intracellular Ca2+ may be elevated by many different ion channels including store-operated channels. It has been further shown that channel blockers may be beneficial in the treatment of neurological motor dysfunction when administered in the acute posttraumatic period (Cheney, Jessica, et al., (2000) J. of Neurotrauma, 17(1):83-91).

2. Inflammatory Disease

Methods provided herein may also be used in connection with treatment of inflammatory diseases. These diseases include but are not limited to asthma, chronic obstructive pulmonary disease, rheumatoid arthritis, inflammatory bowel disease, glomerulonephritis, neuroinflammatory diseases such as multiple sclerosis, and disorders of the immune system.

Vanilloid receptor 1 (VR1) is expressed by sensory neurons. Once activated, these neurons evoke the sensation of burning pain and stimulate the release of neuropeptides that induce neurogenic inflammation. VR1 immunoreactivity is greatly increased in colonic nerve fibers of patients with active inflammatory bowel disease (Yiangou, Y. et al., (2001) Lancet (North American Edition) 357 (9265): 1338-1339). VR1 receptors have also been shown to play a role in airway inflammation (Veronesi, Bellina, et al., (2000) Toxicology and Applied Pharmacology 169 (1): 66-76) and may, thus, be important in the treatment of chronic obstructive pulmonary disease.

The activation of neutrophils (PMN) by inflammatory mediators is partly achieved by increasing cytosolic calcium concentration ((Ca2+)i). Store-operated calcium influx (SOCI) in particular is thought to play an important role in PMN activation. It has been shown that trauma increases PMN SOCI (Hauser, Carl J. et al., (2000) Journal of Trauma Injury Infection and Critical Care 48 (4): 592-598) and that prolonged elevations of (Ca2+)i due to enhanced SOCI may alter stimulus-response coupling to chemotaxins and contribute to PMN dysfunction after injury. Modulation of PMN (Ca2+)i through SOC channels might therefore be useful in regulating PMN-mediated inflammation and spare cardiovascular function after injury, shock or sepsis (Hauser, Carl J. et al., (2001) Journal of Leukocyte Biology 69 (1): 63-68).

3. Cancer

Methods provided herein may also be used in connection with treatment of malignancies, and more particularly those malignancies treatable by non-steroidal anti-inflammatory drugs. These malignancies may including but are not limited to malignancies of lymphoreticular origin, bladder cancer, breast cancer, colon cancer, endometrial cancer, endometrial cancer, head and neck cancer, lung cancer, melanoma, ovarian cancer, prostate cancer and rectal cancer.

Non-steroidal anti-inflammatory drugs (NSAIDs) inhibit proliferation and angiogenesis in colorectal cancer. Weiss et. al. has indicate that store-operated calcium entry may play an important role in cell proliferation in cancer cells and, therefor, inhibition of store-operated calcium entry may contribute to the anti-proliferative effect of non-steroidal anti-inflammatory drugs in human colon cancer cells (Weiss, Helmut, et al., (2001) International Journal of Cancer 92 (6): 877-882). While inhibition of SOCI may aid in the treatment of such cells, the following example shows that other cells of the body may benefit from potentiators SOCI during treatment with NSAIDs.

A major function of prostaglandins is to protect the gastric mucosa. Included in this function is the modulation of store-operated calcium influx in human gastric cells which plays a critical role in cell proliferation. Consequently, inhibition of prostaglandins by nonsteroidal anti-inflammatory drugs (NSAIDs) inhibits store-operated calcium influx in gastric cells (Kokoska, Evan R., et al., (1998) Surgery (St Louis) 124 (2): 429-437). The NSAIDs that relieve inflammation most effectively also produce the greatest gastrointestinal damage (Canadian Family Physician, January 1998, p. 101). Thus, the ability to independently modulate store-operated calcium channels in specific cell types may help to alleviate such side effect of anti inflammatory therapy.

4. Liver Diseases and Disorders

Methods provided herein may also be used in connection with treatment of Liver diseases and disorders. These diseases and disorders include but are not limited to Alcoholic liver disease, liver injury, for example, due to transplantation, hepatitis, cancer, and cirrhosis.

SOCI has been implicated in chronic liver disease (Tao, Jiangchuan, et al., (1999) J. Bio. Chem., 274(34):23761-23769) as well as transplantation injury after cold preservation-warm reoxygenation (Elimadi, Aziz, et al., (2001) Am J. Physiology, 281(3 Part 1): G809-G815. Chronic ethanol consumption has been shown to impair liver regeneration, in part, by modulating SOCI (Zhang, Bao-Hong et al., (1996) J. of Clinical Investigation 98(5):1237-1244).

5. Kidney Diseases and Disorders

Methods provided herein may also be used in connection with treatment of kidney diseases and disorders. Mesangial cell hyperplasia is often a key feature of such diseases and disorders. Such diseases and disorders may be caused by immunological or other mechanisms of injury, including IgAN, membranoproliferative glomerulonephritis or lupus nephritis. Imbalances in the control of mesangial cell replication also appear to play a key role in the pathogenesis of progressive renal failure.

The turnover of mesangial cells in normal adult kidney is very low with a renewal rate of less than 1%. A prominent feature of glomerular/kidney diseases is mesangial hyperplasia due to elevated proliferation rate or reduced cell loss of mesangial cells. When mesangial cell proliferation is induced without cell loss, for example due to mitogenic stimulation mesangioproliferative glomerulonephritis can result. Data has indicated that regulators of mesangial cell growth, particularly growth factors, may act by regulating store-operated calcium channels (Ma, Rong, et al., (2001) J. Am. Soc. of Nephrology, 12:(1) 47-53. Thus, modulators of store-operated calcium influx may aid in the treatment of glomerular diseases by inhibiting mesangial cell proliferation. The epithelial calcium channel CaT2 has also been implicated in hypercalciuria and resultant renal stone formation (Peng, Ji-Bin, et al., (2000) J. Biol. Chem., 275(36):28186-28194).

K. Methods of Identifying Agents for the Treatment of a Disease or Disorder

Disease models are a valuable tool for the discovery and testing of treatment agents. Such disease models may be cellular or organismal and may be produced by methods known to those of skill in the art and described herein. Provided herein are models for diseases and disorders involving or characterized at least in part by calcium dyshomeostasis or alterations in calcium signalling. Also provided herein are methods of identifying candidate agents for the treatment of such diseases and disorders which utilize the models.

1. Cell Models

Cell models for the identification and testing of candidate agents for the treatment of diseases and disorders involving or characterized at least in part by calcium dyshomeostasis or alterations in calcium signalling are provided herein. The cell models can also be used in elucidating the mechanisms underlying calcium dyshomeostasis or altered calcium signalling in a cell as well as in dissecting processes involved in intracellular calcium regulation.

In a particular embodiment, the cell model includes a recombinant cell that contains heterologous nucleic acid encoding one or more proteins involved in intracellular calcium modulation homologous to a protein encoded by a Drosophila gene that, when altered in its expression in a Drosophila cell, results in altered store-operated calcium entry into the cell. An alteration of store-operated ion flux into a cell can be a complete or nearly complete elimination of the activity, a reduction of the activity, an alteration in properties or characteristics (e.g., current properties or sensitivities) of the activity or an increase in the activity relative to the activity in a control cell (e.g., a cell such as a Drosophila cell) that has not been altered in its store-operated ion flux activity. Similarly, an alteration in gene expression may be complete or nearly complete elimination of the expression of a gene, a reduction in the expression of a gene, an increase in the expression of a gene, or an alteration in the protein encoded by the gene (such as truncation or other alteration that effectively renders the protein nonfunctional or provides for aberrant functioning of the protein) relative to the expression of the gene in a cell that has not been altered in its expression of the gene.

In another embodiment, the cell model includes a recombinant cell that contains heterologous nucleic acid encoding one or more proteins involved in intracellular calcium modulation homologous to a protein encoded by a Drosophila gene that, when altered in its expression in a Drosophila cell, results in altered intracellular calcium in the cell. An alteration in intracellular calcium can be any alteration in calcium level, movement, location, or other calcium alteration, in a cell. Thus, in one example, an alteration in intracellular calcium can be an alteration in the calcium level within an intracellular organelle or calcium storage compartment or an alteration in basal or resting cytosolic calcium levels. An alteration of intracellular calcium can be any change in intracellular calcium compared to intracellular calcium in a control cell (e.g., a Drosophila cell that does not have altered expression of the gene). Thus, an alteration of intracellular calcium can be, for example, an increase or decrease in basal or resting cytosolic calcium levels compared to control levels (e.g., levels in a Drosophila cell that does not have altered expression of the gene). Assessment of intracellular calcium can be conducted in a number of ways, such as by methods described herein or known in the art. For example, assessment of basal or resting calcium levels can be conducted as described in Example 3 and by using any methods known in the art. An alteration in basal or resting cytosolic calcium can also be effected in a number of ways, including, but not limited to, alterations in calcium flux across the plasma membrane or membranes of intracellular organelles such as endocytic organelles.

In a further embodiment, the cell model includes a recombinant cell that contains heterologous nucleic acid encoding one or more proteins involved in intracellular calcium modulation homologous to a protein encoded by a Drosophila gene that, when altered in its expression in a Drosophila cell, results in altered store-operated calcium entry into the cell and altered basal or resting cytosolic calcium levels.

In particular embodiments of the above models, the recombinant cell contains heterologous nucleic acid encoding one or more proteins that has an amino acid sequence that is at least about 20%, or at least about 25%, or at least about 30%, or least about 35%, or at least about 40%, or at least about 45%, or at least about 50%, or at least about 52%, or at least about 55%, or at least about 60%, or at least about 65%, or at least about 70%, or at least about 75%, or at least about 80%, or at least about 85%, or at least about 90%, or at least about 95% or more homologous to the protein encoded by the coding sequence of a Drosophila gene that, when altered in its expression, results in altered store-operated calcium entry into the cell and/or altered intracellular calcium. The particular homology can depend on the particular protein, e.g., species, that is homologous to the Drosophila gene, the extent of the encoded Drosophila protein to which the particular protein is homologous, and can also depend on the particular Drosophila gene that, when altered in its expression in a Drosophila cell, results in altered store-operated calcium entry into the cell and/or altered intracellular calcium. In particular embodiments, the protein is at least 52% or more homologous to the protein encoded by the coding sequence of the Drosophila gene. Such exemplary proteins may be homologous to the specified Drosophila protein over at least about 25%, or at least about 30%, or at least about 35%, or at least about 40%, or at least about 45%, or at least about 50%, or at least about 54%, or least about 55%, or at least about 60%, or at least about 65%, or at least about 70%, or at least about 75%, or at least about 80%, or at least about 85%, or at least about 90%, or at least about 95% or more of the protein encoded by the coding sequence of the Drosophila gene. In particular embodiments, the protein is homologous to the Drosophila protein over at least about 54% or more of the protein encoded by the coding sequence of the Drosophila gene.

In another particular embodiment of the above models, the one or more proteins encoded by the heterologous nucleic acid is at least about 52% homologous over at least about 54% of the protein encoded by the coding sequence of the Drosophila gene that, when altered in its expression, results in altered store-operated calcium entry into the cell and/or altered intracellular calcium.

In a particular embodiment, the cell model includes a recombinant cell that contains heterologous nucleic acid encoding one or more proteins homologous to a protein encoded by the coding sequence of Drosophila gene CG4536 or CG5842 as described herein. The particular homology can depend on the particular protein, e.g., species, that is homologous to the protein encoded by Drosophila gene and the extent of the protein encoded by Drosophila gene to which the particular protein is homologous.

Thus, the proteins include those that is (are) at least about 35% homologous to the protein encoded by Drosophila gene CG4536 or CG5842 over at least about 40% of the encoded protein and that provides for store-operated calcium entry. In other embodiments of a protein is homologous to the protein encoded by the Drosophila gene is at least about 35% homologous to the protein encoded by Drosophila gene CG4536 or CG5842 over at least about 50%, 51% or 52% of the encoded protein and that provides for store-operated calcium entry with a test agent or is at least about 41% homologous to the protein encoded by Drosophila gene CG4536 or CG5842 over at least about 50%, 51% or 52% of the encoded protein and that provides for store-operated calcium entry with a test agent. In other embodiments, the protein homologous to the protein encoded by the Drosophila gene is selected to be one that does not contain the contiguous sequence of amino acids EWKFAR (SEQ ID 114) and/or EXD(E)CR(K)GXYXXYE (SEQ ID 115), wherein X is any amino acid and an amino acid residue in parentheses is an alternative to the residue immediately preceding it.

Exemplary proteins are a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp12), a transient receptor potential protein V3 (TRPV3), a stretch-inhibitable nonselective channel (SIC) protein. The protein that provide for store-operated calcium entry includes proteins that are components, such as subunits of a store-operated calcium channel, and includes proteins that affect, directly or indirectly, the activity, expression or level of such components. Also, the proteins can be from any source, particularly mammalian sources, and include human proteins.

In particular embodiments, the cell model includes a recombinant cell that contains heterologous nucleic acid encoding a polymorphic or mutant form of one or more of the above-identified proteins. Such a polymorphic or mutant protein can be one that has an altered activity or function or one that has no activity or is non-functional, particularly relative to a wild-type or predominant form of the protein.

In another embodiment, cell models provided herein for the identification and testing of candidate agents for the treatment of diseases and disorders involving or characterized by calcium dyshomeostasis or altered calcium regulation can include a recombinant cell in which expression of a protein involved in intracellular calcium modulation as identified above, which is either endogenously expressed in the cell or which is expressed in the cell from a heterologous nucleic acid, has been altered or eliminated, such as by replacing or modifying the promoter region or other regulatory region driving expression of nucleic acid encoding the protein. Such a cell can be produced, using methods known in the art and described herein, by introduction into a cell of heterologous nucleic acid that either targets and alters DNA regulatory sequences associated with an endogenous gene or that links DNA encoding the protein to a particular expression regulation sequence(s). Cell models including a recombinant cell in which expression of an endogenous protein involved in intracellular calcium modulation as identified above has been reduced or eliminated can be produced by disruption or elimination (e.g., through gene knock-out, antisense RNA or RNA interference methods) of the gene or RNA encoding the protein in the cell.

In a further embodiment, the cell model includes a recombinant cell that expresses one or more proteins involved in intracellular calcium regulation, such as identified above, as heterologous protein(s). Such cells may overexpress or mis-express the heterologous protein(s). For example, a recombinant cell may be one that endogenously expresses the protein(s) and also has been transfected with additional copies of nucleic acid encoding the protein(s). Alternatively, the host cell used in the generating the recombinant cell may be one that endogenously expresses little to none of the protein(s) of interest.

Any cell may be used in generating the cell models. In particular embodiments of the cell models, the cell is a neuronal, nervous system- or tissue-derived cell or a brain cell. For example, cells from a known cell line can be used, such as from neuroblastoma SH-SY5Y cells, pheochromocytoma PC12 cells, neuroblastoma SK-N-BE(2)C cells, human SK-N-MC neuroblastoma cells, SMS-KCNR cells, human LAN-5 neuroblastoma cells, human GI-CA-N neuroblastoma cells, human GOTO neuroblastoma cells, mouse Neuro 2a (N2A) neuroblastoma cells and/or human IMR 32 neuroblastoma cells. Cell lines include HEK 293, CHO (including CHO-K1), LTK-, N2A, H6, HGB, and Drosophlia S2 cells. The generation, maintenance and use of such cell lines is well known.

2. Animal Models Transgenic animal models and animals for the identification and testing of candidate agents for the treatment of diseases and disorders involving or characterized by calcium dyshomeostasis or altered calcium regulation are provided herein. The animal models can also be used in elucidating the mechanisms underlying calcium dyshomeostasis in an organism as well as in dissecting processes involved in intracellular calcium regulation. Transgenic animals include, but are not limited to, non-human animals, such as rodents (e.g., mice and rats), cows, chickens, pigs, goats, sheep, insects, Drosophila, nematodes, worms, C. elegans, monkeys, gorillas, and other primates.

In particular embodiments, the transgenic non-human animals are such that the expression of nucleic acid encoding one or more proteins as identified above with respect to cell models is altered or eliminated in at least some cells in the animal. Alteration or elimination of expression of the protein(s) can be achieved in a number of ways. For example, expression can be altered by replacing or modifying the promoter region or other regulatory region of an endogenous gene encoding the protein(s) in the animal. Such an animal can be produced by promoting recombination between endogenous nucleic acid and an exogenous nucleic acid.

Increased expression of one or more of the proteins in a transgenic animal can be achieved by altering or replacing an endogenous promoter or by introducing additional copies of nucleic acid encoding the protein(s) into the animal. Reduction or elimination of expression of one or more of the proteins can be achieved by disruption or “knockout” of endogenous genes in the animal. For example, such an animal can initially be produced by promoting homologous recombination between a gene of interest in its chromosome and the corresponding exogenous gene of interest that has been rendered biologically inactive (typically by insertion of a heterologous sequence, e.g., an antibiotic resistance gene). In some organisms, e.g., C. elegans, it is possible to reduce or eliminate expression of a gene encoding a protein by introduction of double-stranded RNA that contains sequence identical or complementary to at least a portion of the sequence of the gene of interest into the animal.

Transgenic animals also include animals containing nucleic acids encoding mutant or polymorphic forms of the one or more proteins. Such animals can be prepared by “knock-in” methods in which the endogenous form (e.g., “normal”) of a gene encoding the protein(s) is replaced by a variant, such a mutant, or other form. It is also possible to replace one species, such as a rodent's, endogenous gene with a gene from another species, such as from a human. Transgenic animals also can be produced by non-homologous recombination into other sites in a chromosome; including animals that have a plurality of integration events.

Introduction of nucleic acids into cells for generation of transgenic animals can be conducted using any known method of nucleic acid delivery, including, but not limited to, microinjection, lipofection and other modes of nucleic acid delivery. The nucleic acids can be introduced into cells such as, for example, germlne cells or somatic cells, such as an embryonic stem cell. For example, the nucleic acid can be introduced into a cell, such as an embryonic stem cell (ES), followed by injecting the ES cells into a blastocyst, and implanting the blastocyst into a foster mother, which is followed by the birth of a transgenic animal. Nuclear transfer methods, in which nucleic acid being used to generate a transgenic animal is introduced into a nuclear donor cell containing a totipotent nucleus, followed by transfer of the donor nucleus into a recipient cell, e.g., an enucleated oocyte, which can be transferred to a recipient female for development into a transgenic animal.

In one method of generating a “knock-out” transgenic animal, homologous recombination is performed by transforming embryo-derived stem (ES) cells with a vector containing the insertionally inactivated gene of interest, such that homologous recombination occurs, followed by injecting the ES cells into a blastocyst, and implanting the blastocyst into a foster mother, followed by the birth of the chimeric animal (“knockout animal”) in which a gene of interest has been inactivated (see Capecchi, Science 244:1288-1292 (1989)). The chimeric animal can be bred to produce homozygous knockout animals, which can then be used to produce additional knockout animals. Knockout animals include, but are not limited to, mice, hamsters, sheep, pigs, cattle, and other non-human mammals. The resulting animals can serve as models of diseases involving altered expression of a protein involved in intracellular calcium modulation. Such knockout animals can be used to screen for candidate therapeutic agents or to test molecules that have already been identified as candidate therapeutic agents for the ability to treat or prevent such diseases or disorders.

3. Evaluation of Models and Effects of Test Agents Thereon

Cell and animal models of diseases and disorders involving calcium dyshomeostasis described herein have a number of uses. For example, by evaluating the cellular or organismal phenotypes associated with the altered expression of proteins involved in intracellular calcium modulation in the cells/organisms and correlating such phenotypes with specific cellular molecules and processes, the disease/disorder models can be used in elucidating the mechanisms underlying calcium dyshomeostasis in a cell as well as in dissecting processes and pathways involved in intracellular calcium regulation. In addition, by evaluating the effects of test agents or candidate therapeutic agents on intracellular calcium and the phenotypic manifestations of the model cells/organisms, the models can be used in screening agents and testing candidate agents for the treatment of diseases and disorders that involve calcium dyshomeostasis.

In methods of screening candidate agents for the treatment of diseases and disorders, the model cells and/or organisms are contacted with a test or candidate agent. Before, during and/or after contacting with a test or candidate agent, intracellular calcium and/or calcium movement is evalulated. For example, resting cytosolic calcium levels and/or store-operated calcium entry in cells can be evaluated. A variety of methods may be used for evaluating such activity and are described herein and known in the art. A test agent that alters intracelluar calcium and/or movement of calcium into, out of or within disease model cells that exhibit calcium dyshomeostasis is identified as an agent that modulates intracellular calcium and possibly as a candidate agent for the treatment of a disease involving calcium dyshomeostasis. An agent that at least partially reverses or reduces or eliminates a disease trait or phenotype exhibited by a model cell or organism, or that tends to restore calcium homeostasis and/or modulates calcium signaling or movement to compensate for disease-associated abnormalities in intracellular calcium is identified as a candidate agent for the treatment of a disease involving calcium dyshomeostasis. A candidate agent for use in treatment of a disease or disorder is thus one that ameliorates or eliminates the symptoms and/or manifestations of an inherited or acquired disease or disorder or one that cures the disease or disorder or at least partially restores the wild-type phenotype. This may include modulation of calcium homeostasis, resting cytosolic calcium levels, and/or store operated calcium entry in model cells and/or organisms.

L. Methods of Treating a Disease or Disorder

Methods provided herein for identifying proteins involved in modulating intracellular calcium as well as agents that modulate intracellular calcium are useful in elucidating cellular processes for calcium homeostasis and signalling. Because the methods can identify proteins, which were not previously known to be involved in intracellular calcium modulation, as calcium-modulating proteins, they are also useful in the discovery of methods for treating diseases and disorders involving calcium dyshomeostasis or altered intracellular calcium regulation.

Provided herein are methods for treating diseases and disorders involving calcium dyshomeostasis or altered intracellular calcium regulation. Such methods can include a step of administering to a subject having a disease or disorder involving calcium dyshomeostasis or altered intracellular calcium regulation, an agent that modulates the level and/or activity of a protein involved in modulation of intracellular calcium. In one embodiment of the methods for treating diseases and disorders, the agent being administered is one that modulates the level and/or activity of a protein that has an amino acid sequence that is homologous to a protein encoded by a Drosophila gene that, when altered in its expression in a Drosophila cell, results in altered store-operated calcium entry into the cell. An alteration of store-operated ion flux into a cell can be a complete or nearly complete elimination of the activity, a reduction of the activity, an alteration in properties or characteristics of the activity or an increase in the activity relative to the activity in a cell that has not been altered in its store-operated ion flux activity. Similarly, an alteration in gene expression may be complete or nearly complete elimination of the expression of a gene, a reduction in the expression of a gene, an increase in the expression of a gene, or an alteration in the protein encoded by the gene (such as truncation or other alteration that effectively renders the protein nonfunctional or provides for aberrant functioning of the protein) relative to the expression of the gene in a cell that has not been altered in its expression of the gene.

In another embodiment of the methods for treating diseases and disorders, the agent being administered is one that modulates the level and/or activity of a protein that has an amino acid sequence that is homologous to a protein encoded by a Drosophila gene that, when altered in its expression in a Drosophila cell, results in altered intracellular calcium in the cell. An alteration in intracellular calcium can be any alteration in calcium level, movement, location, or other calcium alteration, in a cell. Thus, in one example, an alteration in intracellular calcium can be an alteration in the calcium level within an intracellular organelle or calcium storage compartment or an alteration in basal or resting cytosolic calcium levels. An alteration of intracellular calcium can be any change in intracellular calcium compared to intracellular calcium in a control cell (e.g., a Drosophila cell that does not have altered expression of the gene). Thus, an alteration of intracellular calcium can be, for example, an increase or decrease in basal or resting cytosolic calcium levels compared to control levels (e.g., levels in a Drosophila cell that does not have altered expression of the gene). Assessment of intracellular calcium can be conducted in a number of ways, such as by methods described herein or known in the art. For example, assessment of basal or resting calcium levels can be conducted as described in Example 3 and by using any methods known in the art. An alteration in basal or resting cytosolic calcium can also be effected in a number of ways, including, but not limited to, alterations in calcium flux accross the plasma membrane or membranes of intracellular organelles such as endocytic organelles. In a further embodiment of the methods for treating a disease or disorder, the agent being administered is one that modulates the level and/or activity of a protein that has an amino acid sequence that is homologous to a protein encoded by a Drosophila gene that, when altered in its expression in a Drosophila cell, results in altered store-operated calcium entry into the cell and altered basal or resting cytosolic calcium levels.

In particular embodiments of the above methods, the agent being administered is one that modulates the level and/or activity of a protein that has an amino acid sequence that is at least about 20%, or at least about 25%, or at least about 30%, or least about 35%, or at least about 40%, or at least about 45%, or at least about 50%, or at least about 52%, or at least about 55%, or at least about 60%, or at least about 65%, or at least about 70%, or at least about 75%, or at least about 80%, or at least about 85%, or at least about 90%, or at least about 95% or more homologous to the protein encoded by the coding sequence of a Drosophila gene that, when altered in its expression, results in altered store-operated calcium entry into the cell and/or altered intracellular calcium. The particular homology can depend on the particular protein, e.g., species, that is homologous to the Drosophila gene, the extent of the encoded Drosophila protein to which the particular protein is homologous, and can also depend on the particular Drosophila gene that, when altered in its expression in a Drosophila cell, results in altered store-operated calcium entry into the cell and/or altered intracellular calcium. In particular embodiments, the protein is at least 52% or more homologous to the protein encoded by the coding sequence of the Drosophila gene. Such exemplary proteins may be homologous to the specified Drosophila protein over at least about 25%, or at least about 30%, or at least about 35%, or at least about 40%, or at least about 45%, or at least about 50%, or at least about 54%, or least about 55%, or at least about 60%, or at least about 65%, or at least about 70%, or at least about 75%, or at least about 80%, or at least about 85%, or at least about 90%, or at least about 95% or more of the protein encoded by the coding sequence of the Drosophila gene. In particular embodiments, the protein is homologous to the Drosophila protein over at least about 54% or more of the protein encoded by the coding sequence of the Drosophila gene.

In another particular embodiment of the above methods, the protein is at least about 52% homologous over at least about 54% of the protein encoded by the coding sequence of the Drosophila gene that, when altered in its expression, results in altered store-operated calcium entry into the cell and/or altered intracellular calcium.

In another embodiment of the methods for treating a disease or disorder, the agent being administered is one that modulates the level and/or activity of a protein homologous to a protein encoded by the coding sequence of Drosophila gene CG4536 or CG5842 as described throughout the disclosure herein. In particular embodiments of the methods for treating diseases and disorders involving calcium dyshomeostasis or altered intracellular calcium regulation, the agent administered to the subject is one that modulates the level and/or activity of a protein involved in modulation of intracellular calcium that has an amino acid sequence that is at least about 20%, or at least about 25%, or at least about 30%, or least about 35%, or at least about 40%, or at least about 45%, or at least about 50%, or at least about 52%, or at least about 55%, or at least about 60%, or at least about 65%, or at least about 70%, or at least about 75%, or at least about 80%, or at least about 85%, or at least about 90%, or at least about 95% or more homologous to an amino acid sequence of the protein encoded by the coding sequence of at least Drosophila gene CG4536 or CG5842. The particular homology can depend on the particular protein, e.g., species, that is homologous to the protein encoded by the Drosophila gene and the extent of the protein encoded by Drosophila gene to which the particular protein is homologous. In particular embodiments, the protein is at least 52% or more homologous to the protein encoded by the coding sequence of at least Drosophila gene CG4536 or CG5842. Such exemplary proteins may be homologous to the specified Drosophila protein over at least about 25%, or at least about 30%, or at least about 35%, or at least about 40%, or at least about 45%, or at least about 50%, or at least about 54%, or least about 55%, or at least about 60%, or at least about 65%, or at least about 70%, or at least about 75%, or at least about 80%, or at least about 85%, or at least about 90%, or at least about 95% or more of the protein encoded by the coding sequence of Drosophila gene CG4536 or CG5842. In particular embodiments, the protein is homologous to the Drosophila protein over at least about 54% or more of the protein encoded by the coding sequence of CG4536 or CG5842.

In particular, the protein is one that is at least about 35% homologous to the protein encoded by Drosophila gene CG4536 or CG5842 over at least about 40% of the encoded protein and that provides for store-operated calcium entry. In other embodiments the protein is homologous to the protein encoded by the Drosophila gene is at least about 35% homologous to the protein encoded by Drosophila gene CG4536 or CG5842 over at least about 50%, 51% or 52% of the encoded protein and that provides for store-operated calcium entry with a test agent or is at least about 41% homologous to the protein encoded by Drosophila gene CG4536 or CG5842 over at least about 50%, 51% or 52% of the encoded protein and that provides for store-operated calcium entry with a test agent. In other embodiments, the protein homologous to the protein encoded by the Drosophila gene is selected to be one that does not contain the contiguous sequence of amino acids EWKFAR (SEQ ID 114) and/or EXD(E)CR(K)GXYXXYE (SEQ ID 115), wherein X is any amino acid and an amino acid residue in parentheses is an alternative to the residue immediately preceding it.

Exemplary proteins are a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp12), a transient receptor potential protein V3 (TRPV3), a stretch-inhibitable nonselective channel (SIC) protein. The protein that provide for store-operated calcium entry includes proteins that are components, such as subunits of a store-operated calcium channel, and includes proteins that affect, directly or indirectly, the activity, expression or level of such components. Also, the proteins can be from any source, particularly mammalian sources, and include human proteins. In other embodiments, other proteins that may be used in the methods provided herein include, but are not limited to, proteins involved in intracellular calcium modulation that are substantially homologous to the above-listed proteins. In other embodiments, other proteins that may be used in the methods provided herein include, but are not limited to, proteins involved in intracellular calcium modulation that are substantially homologous to the above-listed proteins and mutant or polymorphic forms thereof.

In particular embodiments of the methods for treating diseases and disorders provided herein, the disease or disorder can be, for example, a neurodegenerative disease or disorder, e.g., Alzheimer's disease (AD), Parkinson's disease, Huntington's disease, amyotrophic lateral sclerosis (ALS), and other brain disorders caused by trauma or other insults including aging, an inflammatory disease (e.g., asthma, chronic obstructive pulmonary disease, rheumatoid arthritis, inflammatory bowel disease, glomerulonephritis, neuroinflammatory diseases, multiple sclerosis, and disorders of the immune system), cancer or other proliferative disease, kidney disease and liver disease.

1. Diseases

Because of the important role that calcium regulation plays in many cellular processes including cellular activation, gene expression, cellular trafficking and apoptotic cell death, calcium dyshomeostasis is implicated in the many diseases and disorders involving such cellular activities. These diseases and disorders include neurological, neurodegenerative, inflammatory, liver and kidney diseases and disorders and malignancies, aging-related disorders, and sensitivity to pain and touch.

a. Neurodegenerative Diseases and Disorders

Neurodegenerative diseases and disorders include but are not limited to Alzheimer's disease (AD), Parkinson's disease, Huntington's disease, amyotrophic lateral sclerosis (ALS), and other brain disorders caused by trauma or other insults including aging.

Mechanisms associated with calcium signaling may be altered in many neurodegenerative diseases and in disorders resulting from brain injury. For example, fibroblasts or T-lymphocytes from patients with AD have consistently displayed an increase in Ca²⁺ release from intracellular stores compared to controls [Ito et al. (1994) Proc. Natl. Acad. Sci. U.S.A. 91:534-538; Gibson et al. (1996) Biochem. Biophys. ACTA 1316:71-77; Etchenberrigaray et al. (1998) Neurobiology of Disease, 5:37-45]. Consistent with these observations, mutations in presenilin genes (PS1 or PS2) associated with familial AD (FAD) have been shown to increase InsP3-mediated Ca²⁺ release from internal stores [Guo et al. (1996) Neuro Report, 8:379-383; Leissring et al. (1999) J. Neurochemistry, 72:1061-1068; Leissring et al. (1999) J. Biol. Chem. 274(46):32535-32538; Leissring et al. (2000) J. Biol. Chem. 149(4):793-797; Leissring et al. (2000) Proc. Natl. Acad. Sci. U.S.A. 97(15):8590-8593]. Furthermore, mutations in PS1 or PS2 associated with an increase in amyloidogenic amyloid β peptide generation in AD are reported to be associated with a decrease in store-operated calcium entry [Yoo et al. (2000) Neuron, 27(3):561-572].

Experimental traumatic brain injury has been shown to initiate massive disturbances in Ca²⁺ concentrations in the brain that may contribute to further neuronal damage. Intracellular Ca²⁺ may be elevated by many different ion channels including store-operated channels. It has been further shown that channel blockers may be beneficial in the treatment of neurological motor dysfunction when administered in the acute posttraumatic period [Cheney et al. (2000) J. Neurotrauma, 17(1):83-91].

b. Inflammatory Diseases and Disorders

Methods provided herein may also be used in connection with treatment of inflammatory diseases. These diseases include but are not limited to asthma, chronic obstructive pulmonary disease, rheumatoid arthritis, inflammatory bowel disease, glomerulonephritis, neuroinflammatory diseases such as multiple sclerosis, and disorders of the immune system.

The activation of neutrophils (PMN) by inflammatory mediators is partly achieved by increasing cytosolic calcium concentration ([Ca²⁺]ᵢ). Store-operated calcium influx in particular is thought to play an important role in PMN activation. It has been shown that trauma increases PMN store-operated calcium influx [Hauser et al. (2000) J. Trauma Injury Infection and Critical Care 48 (4):592-598] and that prolonged elevations of [Ca²⁺]ᵢ due to enhanced store-operated calcium influx may alter stimulus-response coupling to chemotaxins and contribute to PMN dysfunction after injury. Modulation of PMN [Ca²⁺]ᵢ through store-operated calcium channels might therefore be useful in regulating PMN-mediated inflammation and spare cardiovascular function after injury, shock or sepsis [Hauser et al. (2001) J. Leukocyte Biology 69 (1):63-68].

C. Cancer and Other Proliferative Diseases

Methods provided herein may also be used in connection with treatment of malignancies, including, but not limited to, malignancies of lymphoreticular origin, bladder cancer, breast cancer, colon cancer, endometrial cancer, head and neck cancer, lung cancer, melanoma, ovarian cancer, prostate cancer and rectal cancer. Store-operated calcium entry may play an important role in cell proliferation in cancer cells [Weiss et al. (2001) International Journal of Cancer 92 (6):877-882].

A major function of prostaglandins is to protect the gastric mucosa. Included in this function is the modulation of store-operated calcium influx in human gastric cells which plays a critical role in cell proliferation. Consequently, inhibition of prostaglandins by nonsteroidal anti-inflammatory drugs (NSAIDs) can inhibit store-operated calcium influx in gastric cells [Kokoska et al. (1998) Surgery (St Louis) 124 (2):429-437]. The NSAIDs that relieve inflammation most effectively also produce the greatest gastrointestinal damage (Canadian Family Physician, January 1998, p. 101). Thus, the ability to independently modulate store-operated calcium channels in specific cell types may help to alleviate such side effect of anti-inflammatory therapy.

d. Liver Diseases and Disorders

Methods provided herein may also be used in connection with treatment of liver diseases and disorders. These diseases and disorders include but are not limited to alcoholic liver disease, liver injury, for example, due to transplantation, hepatitis, cancer, and cirrhosis.

Store-operated calcium entry has been implicated in chronic liver disease [Tao et al. (1999) J. Biol Chem., 274(34):23761-23769] as well as transplantation injury after cold preservation-warm reoxygenation [Elimadi et al. (2001) Am J. Physiology, 281(3 Part 1):G809-G815. Chronic ethanol consumption has been shown to impair liver regeneration, in part, by modulating store-operated calcium entry [Zhang et al. (1996) J. Clin. Invest. 98(5):1237-1244].

e. Kidney Diseases and Disorders

Methods provided herein may also be used in connection with treatment of kidney diseases and disorders. Mesangial cell hyperplasia is often a key feature of such diseases and disorders. Such diseases and disorders may be caused by immunological or other mechanisms of injury, including IgAN, membranoproliferative glomerulonephritis or lupus nephritis. Imbalances in the control of mesangial cell replication also appear to play a key role in the pathogenesis of progressive renal failure.

The turnover of mesangial cells in normal adult kidney is very low with a renewal rate of less than 1%. A prominent feature of glomerular/kidney diseases is mesangial hyperplasia due to elevated proliferation rate or reduced cell loss of mesangial cells. When mesangial cell proliferation is induced without cell loss, for example due to mitogenic stimulation, mesangioproliferative glomerulonephritis can result. Data have indicated that regulators of mesangial cell growth, particularly growth factors, may act by regulating store-operated calcium channels [Ma et al. (2001) J. Am. Soc. of Nephrology, 12:(1) 47-53]. Modulators of store-operated calcium influx may aid in the treatment of glomerular diseases by inhibiting mesangial cell proliferation. The epithelial calcium channel CaT2 has also been implicated in hypercalciuria and resultant renal stone formation [Peng et al. (2000) J. Biol. Chem., 275(36):28186-28194).

2. Agents for Treatment

Agents for use in the methods of treating a disease or disorder can be any substance or combination of substances that modulates the level and/or activity of a protein involved in modulating intracellular calcium as provided herein. Examples of agents include, but are not limited to, small organic molecules, amino acids, peptides, polypeptides, nucleotides, nucleic acids, polynucleotides, carbohydrates, lipids, lipoproteins and glycoproteins. Generally, an agent for treatment of a disease or disorder is a composition, such as a compound or combination of compounds, that when administered to a subject having a disease or disorder effectively reduces, ameliorates or eliminates a symptom or manifestation of the disease or disorder or that cures the disease or disorder. The agent can have such an effect alone or in combination with other agents, or may function to enhance a therapeutic effect of another agent. Examples of agents include nucleic acids, proteins, chemical compounds, carbohydrates or lipids.

3. Methods of Delivering an Agent for Treatment

Agents for use in the methods of treating a disease or disorder as provided herein may be delivered to a subject using any methods known in the art or described herein. Typically, delivery of an agent involves the administration of an effective amount of agent or a pharmaceutically acceptable salt or derivatives thereof. The agent may be administered with a pharmaceutically acceptable, non-toxic, excipient, including solid, semi-solid, liquid or aerosol dosage forms.

Administration of the agent can be via a variety of modes and formulations for administering compounds to subjects. For example, the agent may be administered orally, nasally, intrabronchially, rectally, parenterally, intravascularly, transdermally (including electrotransport), or topically, in the form of a solid, semi-solid, lyophilized powder, or liquid dosage forms, such as, for example, tablets, suppositories, pills, capsules, powders, solutions, suspensions, emulsions, creams, lotions, aerosols, ointments, injectables and gels, preferably in unit dosage forms suitable for simple administration of precise dosages. The compositions typically will include a pharmaceutical carrier or excipient and an active compound (i.e., the agent or pharmaceutically acceptable salt or derivative thereof), and, in addition, may include other medicinal agents, pharmaceutical agent carriers, adjuvants and other such substances.

For oral administration, a pharmaceutically acceptable, non-toxic composition may be formed by the incorporation of excipients, such as, for example, pharmaceutical grades of mannitol, lactose, starch, magnesium stearate, sodium saccharine, talcum, cellulose, glucose, gelatin, sucrose and magnesium carbonate. Such compositions may take several forms, such as solutions, suspensions, tablets, pills, capsules, powders and sustained release formulations. The composition may contain, along with the active ingredient, a diluent, such as lactose, sucrose, dicalcium phosphate, a disintegrant, such as starch or derivatives thereof, a lubricant, such as magnesium stearate, and a binder, such as starch, gum acacia, polyvinylpyrrolidone, gelatin, cellulose and derivatives thereof.

Liquid formulations may, for example, be prepared by dissolving, dispersing an active compound (for example, about 0.1% to about 95%, or 0.1% to about 50%, or about 0.5% to about 20%) and optional pharmaceutical adjuvants in a carrier, such as water, saline, aqueous dextrose, glycerol, and ethanol, to thereby form a solution or suspension. Actual methods of preparing such dosage forms are known, or will be apparent, to those skilled in the art; for example, see Remington's Pharmaceutical Sciences, Mack Publishing Company, Easton, Pa., 15th ed., 1975.

For parenteral administration, the agent may be mixed with a carrier, such as, for example, an oily ester such as ethyl oleate and isopropyl myristate. Sterile liquid pharmaceutical compositions, solutions or suspensions can be utilized by, for example, intraperitoneal injection, subcutaneous injection, intramuscular injection or intravenously.

Transdermal administration of the agent may be conducted through the use of a patch containing the agent and a carrier that is inert to the agent, is non-toxic to the skin and allows delivery of the agent for systemic absorption into the blood stream via the skin. Carriers for transdermal absorption may include pastes, e.g., absorptive powders dispersed in petroleum or hydrophilic petroleum containing the agent with or without a carrier or a matrix containing the agent; creams and ointments, e.g., viscous liquid or semi-solid emulsions, gels and occlusive devices.

Generally, an agent is administered to achieve an amount effective for amelioration of symptoms of the disease or disorder. The dose required to obtain an effective amount may vary depending on the agent, formulation, disease or disorder, and individual to whom the agent is administered.

Determination of effective amounts may also involve in vitro assays in which varying doses of agent are administered to cells in culture and the concentration of agent effective for ameliorating some or all symptoms is determined in order to calculate the concentration required in vivo. Effective amounts may also be based in in vivo animal studies.

The following examples are included for illustrative purposes only and are not intended to limit the scope of the invention.

### EXAMPLE 1

Gene Selection

Candidate intracellular calcium-modulating proteins, and genes encoding such proteins, were selected by searching the annotated Drosophila genome database (GADFLY) (see, e.g., www.fruitfly.org or hedgehog.lbl.gov). Search criteria included annotated molecular function, protein domains containing an ion channel, and primary structures associated with ion transporters. All proteins classified as having homology to domains associated with calcium channels or as having an unknown function were then analyzed for the presence of at least six transmembrane domains by submitting each sequence to the TMpred program (www.ch.embnet.org/software/TMpred_form.html) accessed via the ExPASy Molecular Biology Server (www.expasy.ch).

Genes CG4536 and CG5842 were identified in the search as genes encoding proteins with homology to domains associate with calcium channels. Homologs of these two Drosophila gene products were identified using the ENTREZ Protein search system (accessed via http://www.ncbi.nim.nih.gov/entrez/query.fcgi?db=protein). Each Drosophila gene accession number was entered as a separate query in the ENTREZ protein search command line and, in the results page, the BLink (BLAST Link) feature was used to identify protein across taxonomy that had varying degrees of homology and identity to each Drosophila gene product. The BLink feature provides a display of graphical alignments, editable taxonomic trees, conserved protein classes, protein domains, and 3D structures. A Cut-off value of 250 was used to limit the number of alignments by BLAST score to those proteins having at least 35% homology over at least 40% of the protein.

### EXAMPLE 2

Silencing of Genes CG4536 and CG5842 by RNA Interference (RNAi)

Genes CG 4536 and CG5842 were silenced in S2 cells using RNA interference and the effect monitored on store-operated calcium entry. Cells contacted with each set of RNAi reagents were tested for effects on basal cytosolic calcium levels, vehicle induced calcium entry (DMSO control), or thapsigargin induced calcium entry.

RNAi Protocol

Cell Culture

S2 cells (Invitrogen) were propagated in Drosophila expression system (DES) media (Invitrogen) supplemented with 12.5% FBS, and 200 U/L penicillin/streptomycin. Cells were plated at a density of 1×10⁶ cells/ml until they became ˜90% confluent in a 75-cm² T-flasks. Cells were incubated at 22° C.

dsRNA Production

DNA templates (approximately 700 bp in length) corresponding to the target genes were purified from bacterial stocks (ESTAT26581 for CG4536 and BAC 37.J.22 for CG 5842, from BACPAC Resources, CHORI, Oakland, Calif.) using the Qiagen miniprep system following manufacturers protocols. The target probes were PCR amplified using the EXPAND Long Template PCR system (Roche) as per manufacturer's protocols with the following primers. Each primer used for PCR amplification contained a 5′ T7 RNA polymerase binding site (GAATTAATACGACTCACTATAGGGAGA) (SEQ ID NO: 101) followed by sequences specific for the targeted genes.

| | |
|---|---|
| CG4536 | |
| Upper Primer (T7-162) (44-mer) | |
| GAA TTA ATA CGA CTC ACT ATA GGG AGA CAA CTC GGG CGG ACT GA | (SEQ ID NO:102) |
| Lower Primer (T3-647) (38-mer) | |
| AAT TAA CCC TCA CTA AAG GGA GA TCT TGG CCG GAT TCG | (SEQ ID NO:103) |
| CG5842 | |
| Upper Primer (T7-1010) (43-mer) | |
| GAA TTA ATA CGA CTC ACT ATA GGG AGA AAC GGG AGA TCT ATT G | (SEQ ID NO:104) |
| Lower Primer (T3-1464) (40-mer) | |
| ATT TAA CCC TCA CTA AAG GGA GA CTG CCA TAA TAC GAA GC | (SEQ ID NO:105) |

Double stranded RNA (dsRNA) was generated using the T7 Megascript and T3 Megascript Kits (Ambion, Austin, Tex.). Duplex RNA was achieved by heating equivalent amounts of sense and antisense stands to 65° C. for 30 minutes and then slow cooling to room temperature. The concentration of dsRNA was determined by spectrophotometry (optical density at 260 nm). Double stranded RNA was stored at −20° C.

Conditions for RNAi in Drosophila Cell Culture Drosophila cell culture cells were diluted to a final concentration of 1×106 cells/ml in DES serum free medium. Six milliliters of cells were plated in a 75 cm² T-flask. dsRNA was added directly to the media to an approximate final concentration of 37 nM and mixed by agitation. The cells were incubated for 30 min at room temperature followed by addition of 12 ml of heat inactivated FBS-supplemented DES media. The cells were incubated for an additional 5 days to allow for turnover of the target protein.

Cell Extract Preparation

Cells were harvested by trituration and pelleted by centrifugation at 1000×g. Cells were lysed in 400 μl RIPA buffer (50 mM Tris, pH 8.0; 150 mM NaCl; 1.0% Nonidet P-40; 0.5% Deoxycholate; 0.1% SDS; 0.2 mM NaVO4; 10 mM NaF; 0.4 mM EDTA; 10% glycerol). Lysed cells were centrifuged at 13,000 g for 30 min at 4° C. to pellet cell debris. The supernatant containing the whole cell extract was stored at −20° C.

Analysis of gene silencing by western blot and RT-PCR For control experiments, RNAi mediated gene silencing of peanut (GenBank accession no. P40797; G1730352; see SEQ ID NO: 99 for nucleotide sequence and SEQ ID NO: 100 for an amino acid sequence) was monitored using western blot analysis (Clemens et al. (1996) J. Biol.

Chem. 271:17002-17005) and RT-PCR to determine the extent of gene silencing. For calcium channel experiments, RT-PCR was utilized to monitor gene silencing since the target genes of interest are all novel and presently there are no reagents available to detect the protein products by western blot.

MONITORING CCE IN S2 CELLS AFTER Silencing of SOC Gene Candidates by RNA Interference (RNAi)

CCE was monitored by the methods described in Example 3 and cells were plated into a 96-well plate on day 4 of the RNAi protocol.

### EXAMPLE 3

Measurement of Cytosolic Calcium

Control cells and cells subjected to RNAi for the silencing of CG4536 and CG5842 were analyzed for possible effects of gene silencing on cytosolic calcium. Specifically, basal cytosolic calcium levels and store-operated calcium entry into the cells were evaluated using a fluorescence-based assay.

Monitoring CCE in Cells

CCE channels have specific pharmacological characteristics that are distinct from other ion channels. CCE channels are activated in response to depletion of intracellular calcium stores distinguishing them from other ion channels such as voltage gated channels. CCE can, therefore, be monitored by using store depletion to monitor channel activity. Passive or active store depletion may be used to monitor CCE channel activity. Passive intracellular store depletion can be achieve using a buffering agent such as EGTA or chelator such as TPEN. Active intracellular Ca2+ store depletion can be achieved using inositol-1,4,5-triphosphate (InsP₃) to release Ca²⁺ from the stores directly, or thapsigargin or thapsigargin like compounds such as ionomycin, BHQ, and cyclopiazonic acid may be used to deplete Ca²⁺ from the stores.

FLUO-4 Assay Protocol in S2 Cells

Although the following assay has been carried out with S2 cells, the FLUO-4 assay is a routine assay that may be modified for many different cell types.

S2 cells were seeded in a 96-well plate at a density of 100,000 cells/well (100 μl of 10⁶ cells/ml stock) and allowed to incubate at 22° C. for 1 day. The following day, the media was removed and cells were washed with 2 mM Ca²⁺ buffer (120 mM NaCl, 5 mM KCl, 4 mM MgCl-6H₂O, 2 mM CaCl₂-2H₂O, 10 mM HEPES, pH 7.4). Cells were loaded with 50 μl 10 μM FLUO-4 dye (a CA²⁺-sensitive fluorescent dye) and incubated for 60 minutes at room temperature in the dark. The dye was removed and cells were washed with Ca²⁺ free buffer (120 mM NaCl, 5 mM KCl, 4 mM MgCl-6H₂O, 10 mM HEPES, pH 7.4). Cells were then incubated in 90 μl Ca²⁺ free buffer supplemented with 2.5 mM probenecid. Probenecid inhibits the multidrug resistance transporter, which otherwise would transport loaded dye out of the cell.

Fluorescence of the dye-loaded cells was measured using an excitation wavelength of 485 nm and an emission wavelength of 510 nm on a FluoroSkan fluorimeter (Lab Systems). An Fmax and total fluorescence were measured for each well. The Fmax is the fluorescence of lysed cells that is measured 10 minutes after adding 10 μl of 1% Triton X-100 to each well. The Fmax provides a measure of the relative amount of dye loaded in each well of the 96-well plate, and is proportional to the total number of cells in each well. By taking Fmax into account, any differences in fluorescence of different wells due to well-to-well variability that may arise during the plating procedure can be factored out of the analysis. Total fluorescence is the absolute fluorescence of a well.

An initial reading of basal fluorescence was determined using an excitation wavelength of 485 nm and an emission wavelength of 510 nm on a FluoroSkan fluorimeter (Lab Systems). Cells were then incubated with either 10 μl of 0.1% DMSO (control) or 10 μl of 10 μM thapsigargin for 5 minutes at room termperature. Thapsigargin acts to inhibit the ER Ca²⁺ pump and discharges intracellular Ca 2+stores. In the absence of external Ca²⁺, thapsigargin elicits a transient elevation in intracellular free Ca²⁺ concentration ([Ca²⁺]ᵢ), which is believed to result from release of stored Ca²⁺ followed by removal or buffering of Ca²⁺ back to prestimulation levels. Ten microliters of 20 mM Ca²⁺ buffer (120 mM NaCl, 5 mM KCl, 4 mM MgCl-6H₂O, 20 mM CaCl₂-2H₂O, 10 mM HEPES, pH 7.4) was added and the cells were incubated for 3 minutes at room temperature. Calcium influx was monitored by reading fluorescence as described above.

Evaluation of Basal Cytosolic Calcium

Initial basal, resting calcium levels were determined by measuring the fluorescence of the wells prior to adding any type of agent (e.g., store-depletion agent) or manipulating the medium (e.g., altering the calcium concentration of the medium) in order to evaluate store-operated calcium entry as described below. To determine basal calcium levels, the total fluorescence of a well was divided by the Fmax for that well. The total fluorescence/Fmax values of different wells containing control cells and cells subjected to RNAi to silence gene expression were compared.

The results of these comparisons revealed that the basal, resting cytosolic calcium levels of S2 cells on day 4 after treatment with dsRNA corresponding to the gene sequence were 150% greater than the resting cytosolic calcium levels of control cells. These results identify the protein encoded by the gene as a protein involved in the modulation of intracellular calcium.

Evaluation of Store-Operated Calcium Entry

Store-operated calcium channels have specific pharmacological characteristics that are distinct from other ion channels. Store-operated calcium channels are activated in response to depletion of intracellular calcium stores, which is one feature that distinguishes them from other ion channels such as voltage-gated channels. Store-operated calcium entry can, therefore, be monitored by using store depletion to activate channel activity. Passive or active store depletion may be used to activate store-operated channel activity. Passive intracellular store depletion can be achieved using a buffering agent such as EGTA or chelator such as TPEN. Active intracellular Ca²⁺ store depletion can be achieved using inositol-1,4,5-triphosphate (InsP₃) to release Ca²⁺ from the stores directly. Thapsigargin or thapsigargin-like compounds and compounds such as ionomycin, BHQ, and cyclopiazonic acid may also be used to actively deplete Ca²⁺ from intracellular stores.

Store-operated calcium entry into S2 cells was evaluated in terms of cytosolic calcium levels determined by measuring the fluorescence of the wells after addition of a store-depletion agent. Cells were incubated with either 10 μl of 0.1% DMSO (control) or 10 μl of 10 μM thapsigargin for 5 minutes at room temperature. Thapsigargin acts to inhibit the ER Ca²⁺ pump and discharges intracellular Ca²⁺ stores. In the absence of external Ca²⁺, thapsigargin elicits a transient elevation in cytosolic free Ca²⁺ concentration ([Ca²⁺]ᵢ), which is believed to result from release of stored Ca²⁺, followed by removal or buffering of cytosolic Ca²⁺ back to prestimulation levels. Ten microliters of 20 mM Ca²⁺ buffer (120 mM NaCl, 5 mM KCl, 4 mM MgCI-6H₂O, 20 mM CaCl₂-2H₂O, 10 mM HEPES, pH 7.4) was then added to the cells which were incubated for 3 minutes at room temperature. Cytosolic calcium levels were then evaluated by measuring fluorescence of the wells (both total fluorescence and Fmax) as described above.

To evaluate any cytosolic calcium level increases due to store-operated entry of calcium into the cell from the extracellular medium independently of any increased basal cytosolic calcium levels, the basal fluorescence (measured as described above) was subtracted from the total fluorescence measured after treatment with thapsigarin and incubation in calcium-containing medium. The adjusted fluorescence value was then divided by Fmax for the particular well. These manipulations distinguish an effect on basal calcium from an effect on store-operated calcium entry (i.e., thapsigargin-dependent calcium entry).

### EXAMPLE 4

In Vitro Screening for Agents that Modulate Intracellular Calcium

Cells that contain one or more proteins involved in intracellular calcium modulation, such as the protein encoded by Drosophila gene CG4536 and CG5842 proteins homologous thereto are used to screen for agents that modulate intracellular calcium.

Transfection

Untransfected cells as well as recombinant or transfected cells can be used with any of the methods and process described herein. For generation of recombinant cells, any methods known to those of skill in the art for the insertion of DNA fragments into a vector may be used to construct expression vectors containing nucleic acid encoding an intracellular calcium-modulating protein, and appropriate transcriptional/translational control signals and/or other protein-encoding sequences. Other protein-encoding sequences include nucleic acids encoding ancillary proteins involved in the store-operated calcium entry pathway or calcium homeostasis in general. DNA inserts may include in vitro recombinant DNA and synthetic techniques and in vivo recombinants (genetic recombination).

Nucleic acid encoding an intracellular calcium-modulating protein may be introduced into host cells using a variety of procedures which are known in the art and desribed herein. Transfection methods include, but are not limited to, lipid-mediated transfer and calcium phosphate precipitation; however, the particular procedure used will depend in part on the host cell. Examples of particular transfection procedures include direct uptake using calcium phosphate [CaPO₄; see, e.g., Wigler et al. (1979) Proc. Natl. Acad. Sci. U.S.A. 76:1373-1376], polyethylene glycol [PEG]-mediated DNA uptake, electroporation, lipofection [see, e.g., Strauss (1996) Meth. Mol. Biol. 54:307-327], microcell fusion [see, e.g., Lambert (1991) Proc. Natl. Acad. Sci. U.S.A. 88:5907-5911; U.S. Pat. No. No. 5,396,767, Sawford et al (1987) Somatic Cell Mol. Genet. 13:279-284; Dhar et al. (1984) Somatic Cell Mol. Genet. 10:547-559; and McNeill-Killary et al. (1995) Meth. Enzymol. 254:133-152], lipid-mediated carrier systems [see, e.g., Teifel et al. (1995) Biotechniques 19:79-80; Albrecht et al. (1996) Ann. Hematol. 72:73-79; Holmen et al. (1995) In Vitro Cell Dev. Biol. Anim. 31:347-351; Remy et al. (1994) Bioconjug. Chem. 5:647-654; Le Bolch et al. (1995) Tetrahedron Lett. 3:6681-6684; Loeffler et al. (1993) Meth. Enzymol. 217:599-618].

Suppression of Endogenous Intracellular Calcium-Modulating Proteins

Most cells have some amount of background intracellular calcium-modulating activity. RNAi or gene knockout methods, or compounds known in the art, may be used to suppress this activity. Other methods of suppression include but are not limited to treatment with sense and anti-sense nucleotides and monoclonal antibodies. Such techniques may be accomplished in accordance with any of the known procedures for treating cells to control the production or expression of a selected protein. Such techniques may be implemented prior to or after transfection of cells or may be incorporated into the transfection procedure.

Test Agent Screening Protocol

Agents to be Screened

Any agent can be screened as at test agent.

SH-SY5Y Cell Assay

In an exemplary protocol for conducting an agent screening method, human neuroblastoma cells of cell line SH-SY5Y were evaluated for store-operated calcium entry using fluorescence measurement assays as follows. SH-SY5Y cells were plated in 384-well plates were loaded for 45 min with FLUO-4-AM in a Hanks-buffered salt solution. Cells were washed and placed in a nominally Ca²⁺-free Hanks solution. One minute later, 1 μM thapsigargin (TG) was added to inhibit the ER Ca²⁺ pump and discharge intracellular Ca²⁺ stores. Alternatively, store depletion may be carried out by any of the methods described herein or known in the art. Fifteen minutes after addition of TG, test compound or vehicle was added, followed by another 15 min incubation in Ca²⁺-free buffer. Store-operated calcium entry was then initiated by adding external Ca²⁺ to a final concentration of 1.8 mM and the response monitored using a FLIPR³⁸⁴ (Molecular Devices flourimetric imaging plate reader for high throughput screening) over a period of 10-15 min.

The kinetic data from the FLIPR³⁸⁴ was analyzed and then stored in a relational database (ActivityBase; IDBS). Ten quantitative parameters were calculated that define various aspects of the store-operated calcium entry response. These parameters were as follows:

Mean Basal: basal fluorescence (relative fluorescence units, RFU) readings averaged over 30 seconds prior to addition of Ca²⁺ to initiate store-operated calcium entry

Up slope: linear regression of the increase in RFU from 2 to 60 sec after addition of Ca²⁺

Up rate constant (Up K): the rate constant derived from first-order association of RFUs from 2 seconds to peak response

Peak: the peak RFU (single point) achieved after addition of Ca²+(this parameter, although calculated, is not returned to the database)

Time to peak: the time at which the peak RFU is achieved

Peak/Basal: the difference between peak and mean basal RFU

Decay slope: linear regression of the decrease in RFU from the peak to the end of the measurement period

Decay rate constant (Decay K): the rate constant derived from first-order decay of RFUs from the peak to the end of the measurement period.

Area under the curve (AUC): area under the curve from the addition of Ca²⁺ to the end of the measurement period.

Combinations of these parameters were queried to identify agents that produce at least a 50% change from control. Active agents identified from these queries were retested under identical conditions to confirm their activity. Agents with confirmed activity were then analyzed for concentration-dependent effects, and subsequently, those agents displaying concentration-dependent effects were categorized as confirmed hits.

CHO Cell Assay

CHO cells transfected with DNA encoding wild-type human APP695 and wild-type human presenilin-1 were treated in a manner similar to the SH-SY5Y cells, except that the additions were performed at different times. Cells loaded with FLUO-4, were incubated in nominally Ca²⁺-free buffer and treated with 1 μM TG for 5 minutes prior to addition of vehicle or test compound. Following a five minute incubation with compound or vehicle, store-operated calcium entry is initiated by the addition of 1.8 mM Ca²⁺.

Since modifications will be apparent to those of skill in this art, it is intended that this invention be limited only by the scope of the appended claims.

What is claimed:

1. A method of identifying an agent that modulates intracellular calcium, comprising:
contacting one or more test cells or a portion thereof comprising one or more proteins that is (are) at least about 35% homologous to the protein encoded by Drosophila gene CG4536 or CG5842 over at least about 40% of the encoded protein and that provides for store-operated calcium entry with a test agent, wherein the portion of the cell comprises the one or more proteins;
monitoring the effect(s) of the test compound on store-operated calcium entry; and
identifying a test agent as an agent if it has an effect on store-operated calcium entry.

2. The method of claim 1, wherein the portion of the cell comprises a cell membrane, a cell organelle or a cell organelle membrane.

3. The method of claim 1, wherein:
the step of monitoring the effects, comprises:
comparing store-operated calcium entry into one or more test cell(s) in the presence and absence of the test agent or comparing store-operated calcium entry into a test cell and into a control cell in the presence of the test agent, wherein:
the control cell is substantially similar cell to a test cell, but contains a different amount, including none, of the one or more proteins that is (are) at least about 35% homologous to the protein encoded by Drosophila gene CG4536 or CG5842 over at least about 40% of the encoded protein that the cell comprises; and
the step of identifying comprises identifying a test agent as an agent that modulates intracellular calcium if store-operated calcium entry into the one or more test cells differs in the presence and absence of the test agent or if store-operated calcium entry into a test cell and the control cell differs.

4. The method of claim 1, wherein:
monitoring the effect(s) of the test agent on store-operated calcium entry, comprises:
reducing the calcium level in an intracellular calcium store of one or more test cells; and
monitoring the effect of test agent on intracellular and/or extracellular a) ion movement, b) ion flux or c) ion levels of the one or more test cells; and
identifying comprises identifying a test agent as an agent if it has an effect on a), b) or c), wherein:
the step of reducing the calcium levels in an intracellular calcium store is performed before, simultaneously with or after the step of contacting one or more test cells with the test agent.

5. The method of claim 4, wherein:
monitoring the effect(s) of test agent on store-operated calcium further comprises:
comparing a), b) or c) in the presence and absence of the test agent or comparing a), b) or c) of a test cell and a control cell in the presence of the test agent; and
identifying further comprises:
identifying a test agent as an agent that modulates intracellular calcium if a), b) or c) of a test cell differ in the presence and absence of the test agent or if a), b) or c) of a test cell and the control cell differ, wherein:
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises.

6. The method of claim 4, wherein:
monitoring the effects of test agent on store-operated calcium comprises:
determining intracellular or cytoplasmic calcium levels of one or more test cells; and
identifying comprises:
identifying a test agent as an agent if it has an effect on intracellular or cytoplasmic calcium levels of one or more test cells.

7. The method of claim 6, wherein:
monitoring the effect(s) of test agent on store-operated calcium further comprises:
comparing the intracellular calcium levels of one or more test cells in the presence and absence of the test agent or the intracellular calcium levels of a test cell and of a control cell in the presence of the test agent; and
identifying comprises:
identifying a test agent as an agent that modulates intracellular calcium if the intracellular calcium levels of a test cell differ in the presence and absence of the test agent or if the intracellular calcium levels of a test cell and the control cell differ, wherein
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises.

8. The method of claim 4, wherein:
monitoring the effects of test agent on store-operated calcium comprises:
monitoring intracellular calcium levels over time in one or more test cells for fluctuations in intracellular calcium levels; and
identifying comprises:
identifying a test agent as an agent if it has an effect on intracellular calcium level time courses.

9. The method of claim 8, wherein:
monitoring the effect(s) of test agent on store-operated calcium further comprises:
comparing the intracellular calcium level time courses of a test cell in the presence and absence of the test agent or the intracellular calcium level time courses of a test cell and a control cell in the presence of the test agent; and
identifying comprises:
identifying a test agent as an agent that modulates intracellular calcium if the intracellular calcium level time courses of a test cell differ in the presence and absence of the test agent or if the intracellular calcium level time courses of a test cell and the control cell differ, wherein
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises.

10. The method of claim 4, wherein:
monitoring the effects of test agent on store-operated calcium comprises:
assessing comprises assessing ion flux across the cell plasma membrane of a test cell; and
identifying comprises:
identifying a test agent as an agent if it has an effect on ion flux across the cell plasma membrane of a test cell.

11. The method of claim 10, wherein:
monitoring the effect(s) of test agent on store-operated calcium further comprises:
comparing the ion flux across the cell plasma membrane of a test cell in the presence and absence of the test agent or the ion flux across plasma membrane of a test cell and a control cell in the presence of the test agent, wherein
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises; and
identifying further comprises identifying a test agent as an agent that modulates intracellular calcium if the ion flux across the cell plasma membrane of a test cell differs in the presence and absence of the test agent or if the ion flux across the plasma membrane of a test cell and the control cell differs.

12. The method of claim 1, wherein a protein that is at least about 35% homologous to the protein encoded by Drosophila gene CG4536 or CG5842 over at least about 40% of the encoded protein and that provides for store-operated calcium entry is selected from the group consisting of a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp 12), a transient receptor potential protein V3 (TRPV3), a stretch-inhibitable nonselective channel (SIC) protein.

13. The method of claim 3, wherein a protein that is at least about 35% homologous to the protein encoded by Drosophila gene CG4536 or CG5842 over at least about 40% of the encoded protein and that provides for store-operated calcium entry is selected from the group consisting of a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp12), a transient receptor potential protein V3 (TRPV3), a stretch-inhibitable nonselective channel (SIC) protein.

14. The method of claim 4, wherein a protein that is at least about 35% homologous to the protein encoded by Drosophila gene CG4536 or CG5842 over at least about 40% of the encoded protein and that provides for store-operated calcium entry is selected from the group consisting of a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp12), a transient receptor potential protein V3 (TRPV3) and a stretch-inhibitable nonselective channel (SIC) protein.

15. The method of any of claims 1-10, wherein a protein that is homologous to the protein encoded by the Drosophila gene is at least about 35% homologous to the protein encoded by Drosophila gene CG4536 or CG5842 over at least about 50%, 51% or 52% of the encoded protein and that provides for store-operated calcium entry with a test agent.

16. The method of any of claims 1-10, wherein a protein that is homologous to the protein encoded by the Drosophila gene is at least about 41% homologous to the protein encoded by Drosophila gene CG4536 or CG5842 over at least about 50%, 51% or 52% of the encoded protein and that provides for store-operated calcium entry with a test agent.

17. The method of any of claims 1-10, wherein a protein homologous to the protein encoded by the Drosophila gene does not contain the contiguous sequence of amino acids EWKFAR (SEQ ID 114) and/or EXD(E)CR(K)GXYXXYE (SEQ ID 115), wherein X is any amino acid and an amino acid residue in parentheses is an alternative to the residue immediately preceding it.

18. A method of identifying an agent that modulates intracellular calcium, comprising:
monitoring the effects of an agent on store-operated calcium entry, wherein the agent modulates the activity of, the interaction of, the level of or binds to a protein that provides for store-operated calcium entry and that is at least about 35% homologous to the protein encoded by Drosophila gene CG4536 or CG5842 over at least about 40% of the encoded protein; and
identifying a test agent as an agent that modulates intracellular calcium if it has an effect on store-operated calcium entry.

19. The method of claim 18, wherein the effect on store-operated calcium entry is tested on a test cell or a portion thereof that comprises the protein that is at least about 35% homologous to the protein encoded by Drosophila gene CG4536 or CG5842 over at least about 40% of the encoded protein and that provides for store-operated calcium entry.

20. The method of claim 19, wherein:
the step of monitoring the effects, comprises:
comparing store-operated calcium entry into one or more test cell(s) in the presence and absence of the test agent or comparing store-operated calcium entry into a test cell and into a control cell in the presence of the test agent, wherein:
the control cell is substantially similar cell to a test cell, but contains a different amount, including none, of the one or more proteins that is (are) at least about 35% homologous to the protein encoded by Drosophila gene CG4536 or CG5842 over at least about 40% of the encoded protein that the cell comprises; and
the step of identifying comprises identifying a test agent as an agent that modulates intracellular calcium if store-operated calcium entry into the one or more test cells differs in the presence and absence of the test agent or if store-operated calcium entry into a test cell and the control cell differs.

21. The method of claim 19, wherein:
monitoring the effect(s) of the test agent on store-operated calcium entry, comprises:
reducing the calcium level in an intracellular calcium store of one or more test cells; and
monitoring the effect of test agent on intracellular and/or extracellular a) ion movement, b) ion flux or c) ion levels of the one or more test cells; and
identifying comprises identifying a test agent as an agent if it has an effect on a), b) or c), wherein:
the step of reducing the calcium levels in an intracellular calcium store is performed before, simultaneously with or after the step of contacting one or more test cells with the test agent.

22. The method of claim 21, wherein:
monitoring the effect(s) of test agent on store-operated calcium further comprises:
comparing a), b) or c) in the presence and absence of the test agent or comparing a), b) or c) of a test cell and a control cell in the presence of the test agent, wherein
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises; and
identifying further comprises:
identifying a test agent as an agent that modulates intracellular calcium if a), b) or c) of a test cell differ in the presence and absence of the test agent or if a), b) or c) of a test cell and the control cell differ, wherein:
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises.

23. The method of claim 19, wherein:
monitoring the effects of a test agent on store-operated calcium comprises:
determining intracellular or cytoplasmic calcium levels of one or more test cells; and
identifying comprises:
identifying a test agent as an agent if it has an effect on intracellular or cytoplasmic calcium levels of one or more test cells.

24. The method of claim 23, wherein:
monitoring the effect(s) of test agent on store-operated calcium further comprises:
comparing the intracellular calcium levels of one or more test cells in the presence and absence of the test agent or the intracellular calcium levels of a test cell and of a control cell in the presence of the test agent, wherein
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises; and
identifying comprises:
identifying a test agent as an agent that modulates intracellular calcium if the intracellular calcium levels of a test cell differ in the presence and absence of the test agent or if the intracellular calcium levels of a test cell and the control cell differ.

25. The method of claim 19, wherein:
monitoring the effects of test agent on store-operated calcium comprises:
monitoring intracellular calcium levels over time in one or more test cells for fluctuations in intracellular calcium levels; and
identifying comprises:
identifying a test agent as an agent if it has an effect on intracellular calcium level time courses.

26. The method of claim 25, wherein:
monitoring the effect(s) of test agent on store-operated calcium further comprises:
comparing the intracellular calcium level time courses of a test cell in the presence and absence of the test agent or the intracellular calcium level time courses of a test cell and a control cell in the presence of the test agent, wherein
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises; and
identifying comprises:
identifying a test agent as an agent that modulates intracellular calcium if the intracellular calcium level time courses of a test cell differ in the presence and absence of the test agent or if the intracellular calcium level time courses of a test cell and the control cell differ.

27. The method of claim 31, wherein:
monitoring the effects of test agent on store-operated calcium comprises:
assessing comprises assessing ion flux across the cell plasma membrane of a test cell; and
identifying comprises:
identifying a test agent as an agent if it has an effect on ion flux across the cell plasma membrane of a test cell.

28. The method of claim 27, wherein:
monitoring the effect(s) of test agent on store-operated calcium further comprises:
comparing the ion flux across the cell plasma membrane of a test cell in the presence and absence of the test agent or the ion flux across plasma membrane of a test cell and a control cell in the presence of the test agent; and
identifying further comprises identifying a test agent as an agent that modulates intracellular calcium if the ion flux across the cell plasma membrane of a test cell differs in the presence and absence of the test agent or if the ion flux across the plasma membrane of a test cell and the control cell differs, wherein
a control cell is substantially similar to a test cell, but contains a different amount, including none, of the proteins that is (are) at least about 35% homologous to the protein encoded by Drosophila gene CG4536 or CG5842 over at least about 40% of the encoded protein that the test cell comprises.

29. The method of claim 18, wherein a protein that is at least about 35% homologous to the protein encoded by Drosophila gene CG4536 or CG5842 over at least about 40% of the encoded protein and that provides for store-operated calcium entry is selected from the group consisting of a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp 12), a transient receptor potential protein V3 (TRPV3), a stretch-inhibitable nonselective channel (SIC) protein.

30. The method of claim 1 or claim 18, wherein a protein that provides for store-operated calcium entry is a mammalian protein.

31. The method of claim 30, wherein the mammalian protein is a human protein.

32. The method of claim 18 wherein a protein that is homologous to the protein encoded by the Drosophila gene is at least about 35% homologous to the protein encoded by Drosophila gene CG4536 or CG5842 over at least about 50%, 51% or 52% of the encoded protein and that provides for store-operated calcium entry with a test agent.

33. The method of claim 18, wherein a protein that is homologous to the protein encoded by the Drosophila gene is at least about 41% homologous to the protein encoded by Drosophila gene CG4536 or CG5842 over at least about 50%, 51% or 52% of the encoded protein and that provides for store-operated calcium entry with a test agent.

34. The method of claim 18, wherein a protein homologous to the protein encoded by the Drosophila gene does not contain the contiguous sequence of amino acids EWKFAR (SEQ ID 114) and/or EXD(E)CR(K)GXYXXYE (SEQ ID 115), wherein X is any amino acid and an amino acid residue in parentheses is an alternative to the residue immediately preceding it.

35. The method of claim 1 or claim 18, wherein the protein that provides for store-operated calcium entry is a component of a store-operated calcium entry channel.

36. The method of claim 1 or claim 18, wherein the proteins that is (are) at least about 35% homologous to the protein encoded by a Drosophila gene over about 40% of the encoded protein are selected from among ion transport proteins.

37. The method of claim 1 or claim 18, wherein the cell is a recombinant cell and at least one of the one or more proteins that is (are) at least about 35% homologous to a protein encoded by a Drosophila gene over about 40% of the encoded protein is heterologous to the cell.

38. The method of claims 1 or claim 18, wherein the test cell is a mammalian cell.

39. The method of claims 37, wherein the test cell is a mammalian cell.

40. The method of claim 1 or claim 18, wherein the agent is a drug for treatment of a neurodegenerative disease.

41. The method of claim 1 or claim 18, wherein a protein that is at least about 35% homologous to the protein encoded by Drosophila gene CG4536 or CG5842 over at least about 40% of the encoded protein and that provides for store-operated calcium entry is a calcium transport protein 1 (CaT protein) or an ECaC protein.

42. A method of modulating store-operated calcium entry, comprising modulating the level of, expression of, activity of or molecular interactions of a protein in a cell that has altered store-operated calcium entry, wherein the protein is at least about 35% homologous to the protein encoded by Drosophila gene CG4536 or CG5842 over at least about 40% of the encoded protein and that provides for store-operated calcium entry, and whereby store-operated calcium transport into the cell is modulated.

43. The method of claim 42, wherein a protein that is at least about 35% homologous to the protein encoded by Drosophila gene CG4536 or CG5842 over at least about 40% of the encoded protein and that provides for store-operated calcium entry is selected from the group consisting of a calcium transport protein 1 (CaT protein), an ECaC protein, an epithelial calcium channel, a CaT2 protein, an olfactory channel, a stretch-activated channel protein, a vanilloid receptor-related osmotically activated channel protein, a vanilloid receptor type 1-like protein, a vanilloid receptor subtype 1 protein, a vanilloid receptor 1 protein, a vanilloid receptor-like protein, a capsaicin receptor protein, an olfactory trp C4 (OTRPC4) protein, a growth factor-regulated calcium channel protein, a transient receptor potential protein 12 (trp12), a transient receptor potential protein V3 (TRPV3), a stretch-inhibitable nonselective channel (SIC) protein.

44. The method of claim 43, wherein the protein is a CaT1 or EcAc protein.

45. A method of identifying a molecule that provides for store-operated calcium entry, comprising identifying a molecule that interacts with a protein that is least about 35% homologous to the protein encoded by Drosophila gene CG4536 or CG5842 over at least about 40% of the encoded protein and that provides for store-operated calcium entry, thereby identifying molecules involved in modulating store-operated calcium entry.

46. The method of claim 45, further comprising eliminating, altering or reducing the expression of a gene encoding the protein in a cell and evaluating intracellular calcium.

47. The method of claim 45, wherein the protein that is least about 35% homologous to the protein encoded by Drosophila gene CG4536 or CG5842 over at least about 40% of the encoded protein is a mammalian protein.

48. The method of claim 46, wherein the cell in which intracellular calcium is evaluated is a mammalian cell.
